(19) United States
(12) Patent Application Publication
(10) Pub. No.: US 2021/0196823 A1
(43) Pub. Date: Jul. 1, 2021

(54) Methods of Treating Metabolic Disease

(71) Applicant: Pfizer Inc., New York, NY (US)

(72) Inventors: Alexander Hal Drakesmith, Oxford (GB); João André Traila Arezes, Oxford (GB); Simon John Draper, Oxford (GB); Kirsty Anne McHugh, Oxford (GB); Fredrik Karpe, Oxford (GB); Nathan Denton, Oxford (GB); Orla Cunningham, Dublin (IE); Niall John Foy, Dublin (IE); Reema Jasuja, Cambridge, MA (US)

(21) Appl. No.: 17/250,083

(22) PCT Filed: Jun. 6, 2019
(86) PCT No.: PCT/IB2019/054720

§ 371 Date: Nov. 23, 2020

Related U.S. Application Data
(60) Provisional application No. 62/682,309, filed on Jun. 8, 2018.

(51) Int. Cl. A61K 39/395; C07K 16/26; C07K 16/24; A61K 45/06; A61K 31/426; A61K 31/522; A61K 31/366; A61K 31/4985; A61K 38/28; A61P 1/16; A61P 3/04; A61P 3/10

(52) U.S. Cl. CPC A61K 39/3955; C07K 2317/76; C07K 16/241; A61K 45/06; A61K 31/426; A61K 31/522; A61K 31/366; A61K 31/4985; A61K 38/28; A61P 1/16; A61P 3/04; A61P 3/10; C07K 2317/51; C07K 2317/515; C07K 2317/565; C07K 2317/75; C07K 16/26

(57) ABSTRACT

The present invention further relates to methods for treating a disease of iron metabolism and disease of fat or carbohydrate metabolism using a BMP agonist or antagonist

The present invention relates to BMPs (bone morphogenetic proteins) which regulate metabolic homeostasis of iron, fats and carbohydrates. In particular, the present invention relates to methods for treating a disease of iron metabolism or a disease comprising abnormally high or low hepcidin levels or abnormally high or abnormally low iron levels using a BMP agonist or antagonist. The present invention further relates to methods for treating a disease of fat or carbohydrate metabolism using a BMP agonist or antagonist. The present invention further relates to methods for treating a disease of iron metabolism or treating a disease of fat or carbohydrate metabolism by inhibition of the interaction between BMPs and erythroferrone (ERFE)/FAM132b.

### BACKGROUND OF THE INVENTION

Iron is essential for erythropoiesis. Enhanced iron availability is required for recovery from hemorrhage, but excess iron is pathological as for example in β-thalassemia. Iron absorption is tightly regulated by erythropoietic demand via control of hepcidin expression. Hepcidin inhibits the cellular iron exporter ferroportin, preventing iron export from ferroportin-expressing cells, thus reducing iron recycling through splenic macrophages and uptake of dietary iron through enterocytes. When iron is in high demand, following acute blood loss or due to hypoxia, hepcidin is suppressed to allow iron mobilization for increased erythropoiesis. Hepcidin expression is modulated via the BMP/SMAD signalling pathway. BMP6 and BMP2, produced by liver sinusoidal endothelial cells, trigger a signalling cascade by binding to BMP receptors on hepatocyte cell membranes, which phosphorylate cytosolic SMADs (SMAD1/5/8) that translocate to the nucleus complexed with SMAD4 to activate the transcription of target genes, including hepcidin (HAMP). Because of the key role of hepcidin deficiency or hepcidin excess in the pathogenesis of various iron disorders, agonists or antagonists of hepcidin activity would be expected to improve the treatment of such disorders. Agonists of hepcidin activity should be useful for treating iron overload such as in hereditary hemochromatosis and in thalassemia, likewise antagonists in the case of anemias. BMP pathway inhibition or activation should offer a selective means for achieving liver hepcidin regulatory pathway control. Erythropoietin (EPO) causes hepcidin suppression at least in part by increasing synthesis of the hormone erythroferrone (ERFE). Erythropoietin (EPO) enhances erythroferrone (ERFE) synthesis by erythroblasts, and ERFE suppresses expression of hepcidin in the liver, thereby increasing iron levels. ERFE is produced by erythroblasts after bleeding or EPO treatment, and acts on hepatocytes to suppress hepcidin expression and increase iron availability. However, the mechanism by which ERFE suppresses hepcidin is still unknown. We found that EPO suppressed hepcidin and hepatic BMP/SMAD pathway genes in vivo in a partially ERFE-dependent manner. Recombinant ERFE also suppressed hepatic BMP/SMAD pathway independently of changes in serum and liver iron, and in vitro, ERFE decreased SMAD 1/5/8 phosphorylation. ERFE specifically inhibited stimulation of hepcidin induction by BMP5, BMP6 and BMP7, leading to hepcidin suppression. This effect appears to be mediated through the direct binding interaction between BMP and ERFE. BMPs are also implicated in fat metabolism, recent studies suggested BMP2/SMAD6 might be involved in both adipose and insulin biology relating to body fat distribution, (Shungin et al, Nature, 2015), BMP2 and BMP6 have also been found to ameliorate insulin resistance (Schreiber et al, Sci Rep, 2017). We have determined that ERFE binds to and affects the activity of both BMP2 and BMP6, modulation of this interaction therefore offers a means to provide an impact in improving insulin tolerance and ameliorate various aspects of impaired body fat distribution, such as the development of diabetes, metabolic and non-alcoholic fatty liver disease.

### SUMMARY OF THE INVENTION

Treatment of a Disease of Iron Metabolism

According to a first aspect of the present invention there is provided a method of treating, preventing, ameliorating, controlling, reducing incidence of, or delaying the development or progression of the development or progression of a disease of iron metabolism using a BMP agonist or antagonist. The disease of iron metabolism may be a disease comprising abnormally high or low iron levels, a disease comprising abnormally high or low hepcidin levels, a disease comprising abnormally high or low hepcidin activity. The disease of iron metabolism may be a disease comprising abnormally high hepcidin levels and/or activity and/or abnormally low iron levels. The disease of iron metabolism may be a disease comprising abnormally low hepcidin levels and/or activity and/or abnormally high iron levels. The disease of iron metabolism can be anemia, for example iron-deficiency anemia, iron-refractory iron deficiency anemia, anemia of chronic kidney disease, parasitic anemia, malarial anemia; or thalassemia, for example beta-thalassemia. According to the invention the level, which includes concentration, or activity, can be that present and/or measured in a biological sample. Accordingly the present invention provides a method of treating, preventing, ameliorating, controlling, reducing incidence of, or delaying the development or progression of the development or progression of parasitemia, for example parasitemia associated with parasitic anemia for example malarial anemia, using a BMP agonist or antagonist. Accordingly there is also provided a method of treating a disease comprising abnormally low hepcidin levels and/or activity and/or abnormally high iron levels using a BMP agonist. Accordingly there is also provided a method of treating a disease comprising abnormally high hepcidin levels and/or activity and/or abnormally low iron levels using a BMP antagonist.

A disease or disorder of iron metabolism and/or disease or disorder comprising abnormally low or high hepcidin levels, amounts or expression may be determined by those skilled in the art using methods known in the art such as the assays to determine and monitor hepcidin levels and expression or iron levels presented in WO 2004092405 or in U.S. Pat. No. 7,534,764 and as disclosed herein.

Diseases of iron metabolism include hemochromatosis, such as HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis. Diseases of iron metabolism also include myelodysplasia syndrome, hepcidin deficiency, transfusional iron overload, thalassemia, for example thalassemia such as thalassemia intermedia, alpha thalassemia, beta thalassemia, delta thalassemia. Diseases of iron metabolism also include sideroblastic anemia, porphyria, porphyria cutanea tarda, African iron overload, hyperferritinemia, ceruloplasmin deficiency, atransferrinemia. Diseases of iron metabolism additionally include anemia, for example congenital dyserythropoietic anemia, anemia of chronic disease, anemia of inflammation, anemia of infection, hypochromic microcytic anemia, iron-deficiency anemia, iron-refractory iron deficiency anemia, anemia of chronic kidney disease, parasitic anemia, malarial anemia. Diseases of iron metabolism further include erythropoietin resistance, iron deficiency of obesity, benign or malignant tumors that overproduce hepcidin or induce its overproduction, conditions with hepcidin excess, Friedreich ataxia, gracile syndrome, Hallervorden-Spatz disease, Wilson's disease, pulmonary hemosiderosis, hepatocellular carcinoma, cancer, hepatitis, cirrhosis of liver, pica, chronic renal failure, insulin resistance, diabetes, diabetes Type I or diabetes Type II, insulin resistance, glucose intolerance, atherosclerosis, neurodegenerative disorders, multiple sclerosis, Parkinson's disease, Huntington's disease, and Alzheimer's disease.

Treatment of Disease of Lipid or Carbohydrate Metabolism

According to a second aspect of the present invention there is provided a method of treating, preventing, ameliorating, controlling, reducing incidence of, or delaying the development or progression of the development or progression of a disease of lipid or carbohydrate metabolism using a BMP agonist or antagonist.

The disease of lipid or carbohydrate metabolism may be or comprise a disease comprising impaired body fat distribution, insulin intolerance or resistance, low insulin level, high blood sugar, high serum triglycerides, low high-density lipoprotein (HDL) level, steatosis, fibrosis and/or cirrhosis of the liver, high blood pressure, or cardiovascular disease. The disease of lipid or carbohydrate metabolism may be or comprise non-alcoholic fatty liver disease (NAFLD), non-alcoholic steatohepatitis (NASH), pediatric nonalcoholic fatty liver disease (NAFLD), pediatric non-alcoholic steatohepatitis (NASH), optionally wherein the disease further comprises obesity, diabetes, high cholesterol or high triglycerides, metabolic syndrome. The disease of lipid or carbohydrate metabolism may be diabetes, type 1 diabetes mellitus, type 2 diabetes mellitus, gestational diabetes, prediabetes, optionally wherein the disease further comprises obesity, high cholesterol or high triglycerides, NASH, NAFLD.

Accordingly there is provided a method of treating a disease of lipid metabolism using a BMP agonist or antagonist.

Accordingly there is provided a method of treating a disease of carbohydrate metabolism using a BMP agonist or antagonist. Accordingly there is provided a method of treating NASH using a BMP agonist or antagonist. Accordingly there is provided a method of treating NAFLD using a BMP agonist or antagonist. Accordingly there is provided a method of treating obesity using a BMP agonist or antagonist. Accordingly there is provided a method of treating abnormally high cholesterol level using a BMP agonist or antagonist. Accordingly there is provided a method of treating abnormally high triglyceride level using a BMP agonist or antagonist. Accordingly there is provided a method of treating diabetes using a BMP agonist or antagonist. Accordingly there is provided a method of treating diabetes type 1 using a BMP agonist or antagonist. Accordingly there is provided a method of treating diabetes type 2 using a BMP agonist or antagonist. Accordingly there is provided a method of treating metabolic syndrome using a BMP agonist or antagonist.

Non-alcoholic fatty liver disease (NAFLD) is the build up of excess fat in liver cells that is not caused by alcohol. The more severe form of non-alcoholic fatty liver disease is called non-alcoholic steato hepatitis (NASH) and causes the liver to swell and become damaged. NASH tends to develop in people who are overweight or obese, or have diabetes, high cholesterol or high triglycerides. Non-alcoholic steato hepatitis is one of the leading causes of cirrhosis in adults. Metabolic syndrome comprises at least three, i.e. three or more of the five following medical conditions: abdominal obesity, high blood pressure, high blood sugar, high serum triglycerides and low high-density lipoprotein (HDL) levels. Insulin resistance, metabolic syndrome, and prediabetes are closely associated with metabolic syndrome and obesity. Metabolic syndrome is particularly associated with increased risk of developing cardiovascular disease and type 2 diabetes and is prevalent in about a quarter of the adult US population.

BMP Agonists or Antagonists

According to either aspect of the present invention the BMP agonist or antagonist may be an agonist or antagonist of any one or more of BMP 2, 2/6 heterodimer, 3, 4, 5, 6, 7, 8a, 8b, 9, 10, 11, 12, 13, 14, or 15. The BMP agonist or antagonist may be an agonist or antagonist of, BMP2, BMP2/6 heterodimer, BMP4, BMP5, BMP6 or BMP7. The BMP agonist or antagonist may be an agonist or antagonist of BMP2/6 heterodimer, BMP5, BMP6 or BMP7. The BMP agonist or antagonist may be an agonist or antagonist of BMP5, BMP6 or BMP7. The BMP agonist or antagonist may be an agonist or antagonist of BMP2, BMP2/6 heterodimer, or BMP6, an agonist or antagonist of BMP2 or BMP6, an agonist or antagonist of BMP2. The BMP agonist or antagonist may be an agonist or antagonist of BMP activity. In particular according to the first aspect of the present invention the BMP agonist or antagonist may be an agonist or antagonist of BMP2, BMP2/6 heterodimer, BMP5, BMP6 or BMP7, preferably BMP5, BMP6 or BMP7; and according to the second aspect the BMP agonist or antagonist may be an agonist or antagonist of BMP2 or BMP6, alternatively BMP 2 or BMP 2/6 or BMP 4. According to the present invention the BMP agonist or antagonist can agonise or antagonise the biological activity, BMP activity or activity of BMP. BMP can be any one of BMP 2, 2/6 heterodimer, 3, 4, 5, 6, 7, 8a, 8b, 9, 10, 11, 12, 13, 14, or 15. According to the present invention the BMP agonist or antagonist can inhibit the activity of an agonist or antagonist of BMP or can inhibit or enhance the binding or interaction of BMP with its receptor.

According to the present invention the BMP agonist or antagonist can agonise or antagonise BMP activity by binding to and/or activating or inhibiting BMP, or BMP polypeptide having BMP activity. According to the present invention the BMP agonist or antagonist can agonise or antagonise BMP activity by preventing or inhibiting the interaction between BMP or BMP polypeptide having BMP activity and a BMP agonist or antagonist or by preventing or inhibiting the interaction between BMP or BMP polypeptide having BMP activity with a BMP agonist or antagonist or with ERFE or ERFE polypeptide having erythroferrone activity, or between BMP or BMP polypeptide having BMP activity and a BMP receptor. According to the present invention the BMP agonist or antagonist can agonise or antagonise BMP activity by enhancing the interaction between BMP or BMP polypeptide having BMP activity and an agonist or antagonist or by enhancing the interaction between BMP or BMP polypeptide having BMP activity and ERFE or ERFE polypeptide having erythroferrone activity, or between BMP or BMP polypeptide having BMP activity and a BMP receptor. According to the present invention the BMP agonist or antagonist can agonise or antagonise BMP activity by enhancing the interaction between BMP or BMP polypeptide having BMP activity and an agonist or antagonist or by enhancing the interaction between BMP or BMP polypeptide having BMP activity and ERFE or ERFE polypeptide having erythroferrone activity, or between BMP or BMP polypeptide having BMP activity and a BMP receptor.

According to the present invention the BMP agonist or antagonist can agonise or antagonise BMP activity by inhibiting the action of an agonist or antagonist of BMP or BMP polypeptide having BMP activity for example by (i) binding to BMP, or a BMP polypeptide having BMP activity, and preventing its interaction with and/or inhibition or activation by an agonist or antagonist or (ii) binding to an agonist or antagonist of BMP or BMP polypeptide having BMP activity and preventing its interaction with and/or inhibition or activation of BMP or BMP polypeptide having BMP activity (iii) binding to BMP, or a BMP polypeptide having BMP activity, and preventing its interaction with and/or inhibition by ERFE or ERFE polypeptide having erythroferrone activity (iv) binding to ERFE or an ERFE polypeptide having erythroferrone activity and preventing or inhibiting its interaction with BMP or BMP polypeptide having BMP activity and/or inhibition of BMP activity. According to the present invention the BMP agonist or antagonist can agonise or antagonise BMP activity by (i) binding to BMP or a BMP polypeptide having BMP activity and preventing or inhibiting its interaction with a BMP receptor, (ii) binding to BMP or a BMP polypeptide having BMP activity and enhancing its interaction with a BMP receptor, (iii) binding to a BMP receptor and preventing or inhibiting its interaction with its BMP or BMP polypeptide having BMP activity, (iv) binding to a BMP receptor and enhancing its interaction with its BMP or BMP polypeptide having BMP activity; whereby the activity mediated by BMP binding to the BMP receptor is agonised or antagonised.

According to the invention the BMP agonist or antagonist can specifically bind to a BMP or a BMP polypeptide having BMP activity, preferably (i) BMP2, (ii) BMP2/6 heterodimer, (iii) BMP4, (iv) BMP5, (v) BMP6 or (vi) BMP7 with a binding constant or KD of about or less than about 0.001 nM, preferably of about or less than about 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 325, 350, 375, 400, 425, 450, 475, 500, 525, 550, 575, 600, 625, 650, 675, 700, 725, 750, 775, 800, 825, 850, 875, 900, 925, 950, 975, 1000 nM, +/−5% or 10% error as measured in a suitable activity assay such as for example an SPR (surface plasmon resonance) or HTRF (Homogeneous Time Resolved Fluorescence) assay for example as described herein.

According to the invention the BMP agonist or antagonist can specifically bind to (a) an agonist of BMP or BMP polypeptide having BMP activity, (b) an antagonist of BMP or BMP polypeptide having BMP activity, (c) a BMP receptor, (d) ERFE or ERFE polypeptide having erythroferrone activity; with a binding constant or KD of about or less than about 0.001 nM, preferably of about or less than about 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 325, 350, 375, 400, 425, 450, 475, 500, 525, 550, 575, 600, 625, 650, 675, 700, 725, 750, 775, 800, 825, 850, 875, 900, 925, 950, 975, 1000 nM, +/−5% or 10% error as measured in a suitable activity assay such as for example an SPR (surface plasmon resonance) or HTRF (Homogeneous Time Resolved Fluorescence) assay for example as described herein. Preferably the BMP receptor is a receptor of (i) BMP2, (ii) BMP2/6 heterodimer, (iii) BMP4, (iv) BMP5, (v) BMP6 or (vi) BMP7.

According to the invention the BMP agonist or antagonist can specifically inhibit the binding of BMP or a BMP polypeptide having BMP activity, preferably (i) BMP2, (ii) BMP2/6 heterodimer, (iii) BMP4, (iv) BMP5, (v) BMP6 or (vi) BMP7 to any one or more of (a) an agonist of BMP or BMP polypeptide having BMP activity, (b) an antagonist of BMP or BMP polypeptide having BMP activity, (c) a BMP receptor, (d) ERFE or ERFE polypeptide having erythroferrone activity, with an IC50 or inhibition constant (Ki) of about or less than about 0.001 nM, preferably of about or less than about 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 325, 350, 375, 400, 425, 450, 475, 500, 525, 550, 575, 600, 625, 650, 675, 700, 725, 750, 775, 800, 825, 850, 875, 900, 925, 950, 975, 1000 nM, +/−5% or 10% error as measured in a suitable activity assay such as for example an SPR (surface plasmon resonance) or HTRF (Homogeneous Time Resolved Fluorescence) assay for example as described herein. Preferably the BMP receptor is a receptor of (i) BMP2, (ii) BMP2/6 heterodimer, (iii) BMP4, (iv) BMP5, (v) BMP6 or (vi) BMP7.

According to the invention the BMP agonist or antagonist can specifically enhance the binding of BMP or a BMP polypeptide having BMP activity, preferably (i) BMP2, (ii) BMP2/6 heterodimer, (iii) BMP4, (iv) BMP5, (v) BMP6 or (vi) BMP7 to any one or more of (a) an agonist of BMP or BMP polypeptide having BMP activity, (b) an antagonist of BMP or BMP polypeptide having BMP activity, (c) a BMP receptor, (d) ERFE or ERFE polypeptide having erythroferrone activity and improve the binding affinity (KD) of the interaction by about any of about 2, 4, 6, 8, 10, 12, 14, 16, 18, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 800, 825, 850, 875, 900, 925, 950, 975, 1000, 2000, 3000, 4000, 5000, 6000, 7000, 8000, 9000, 10,000 times, for example as measured in a suitable activity assay such as for example an SPR (surface plasmon resonance) for example as described herein. Preferably the BMP receptor is a receptor of (i) BMP2, (ii) BMP2/6 heterodimer, (iii) BMP4, (iv) BMP5, (v) BMP6 or (vi) BMP7.

According to the present invention the BMP agonist or antagonist can bind specifically or selectively to BMP or a BMP polypeptide having BMP activity, can bind specifically or selectively to an agonist or antagonist of BMP or BMP polypeptide having BMP activity, can bind specifically or selectively to ERFE or an ERFE polypeptide having erythroferrone activity, can bind specifically or selectively to a BMP receptor; whereby agonism or antagonism of BMP activity is mediated.

According to the invention the BMP agonist or antagonist can selectively bind to BMP or a BMP polypeptide having BMP activity preferably (i) BMP2, (ii) BMP2/6 heterodimer, (iii) BMP4, (iv) BMP5, (v) BMP6 or (vi) BMP7 in comparison to another different BMP family member selected from the group of 2, 2/6 heterodimer, 3, 4, 5, 6, 7, 8a, 8b, 9, 10, 11, 12, 13, 14, or 15; preferably wherein, the binding affinity (KD) of the agonist or antagonist for the BMP or a BMP polypeptide having BMP activity is between about 2 and 10,000 times tighter than the KD for the other selected BMP family member(s). Preferably the binding affinity can be greater by any of about 2, 4, 6, 8, 10, 12, 14, 16, 18, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 800, 825, 850, 875, 900, 925, 950, 975, 1000, 2000, 3000, 4000, 5000, 6000, 7000, 8000, 9000, 10,000 times tighter, for example as measured in a suitable activity assay such as for example an SPR (surface plasmon resonance) for example as described herein. Preferably the BMP receptor is a receptor of (i) BMP2, (ii) BMP2/6 heterodimer, (iii) BMP4, (iv) BMP5, (v) BMP6 or (vi) BMP7.

According to the invention the BMP agonist or antagonist can selectively inhibit the binding of BMP or a BMP polypeptide having BMP activity, preferably (i) BMP2, (ii) BMP2/6 heterodimer, (iii) BMP4, (iv) BMP5, (v) BMP6 or (vi) BMP7 to any one or more of (a) an agonist of BMP or BMP polypeptide having BMP activity, (b) an antagonist of BMP or BMP polypeptide having BMP activity, (c) a BMP receptor, (d) ERFE or ERFE polypeptide having erythroferrone activity, in comparison to another different BMP family member selected from the group of 2, 2/6 heterodimer, 3, 4, 5, 6, 7, 8a, 8b, 9, 10, 11, 12, 13, 14, or 15; preferably wherein, the binding affinity (KD) is between about 2 and 10,000 times weaker in comparison to the KD for the other selected BMP family member. Preferably the binding affinity (KD) can be weaker by about 2, 4, 6, 8, 10, 12, 14, 16, 18, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 800, 825, 850, 875, 900, 925, 950, 975, 1000, 2000, 3000, 4000, 5000, 6000, 7000, 8000, 9000, 10,000 times weaker, for example as measured in a suitable activity assay such as for example an SPR (surface plasmon resonance) for example as described herein. Preferably the BMP receptor is a receptor of (i) BMP2, (ii) BMP2/6 heterodimer, (iii) BMP4, (iv) BMP5, (v) BMP6 or (vi) BMP7.

According to the invention the BMP agonist or antagonist can selectively enhance the binding of BMP, preferably can selectively enhance the binding of BMP or a BMP polypeptide having BMP activity preferably (i) BMP2, (ii) BMP2/6 heterodimer, (iii) BMP4, (iv) BMP5, (v) BMP6 or (vi) BMP7 to any one or more of (a) an agonist of BMP or BMP polypeptide having BMP activity, (b) an antagonist of BMP or BMP polypeptide having BMP activity, (c) a BMP receptor, (d) ERFE or ERFE polypeptide having erythroferrone activity, in comparison to another different BMP family member selected from the group of 2, 2/6 heterodimer, 3, 4, 5, 6, 7, 8a, 8b, 9, 10, 11, 12, 13, 14, or 15; preferably wherein, the binding affinity (KD) is between about 2 and 10,000 times tighter in comparison to the KD for the other selected BMP family member. Preferably the selectivity according to binding affinity (KD) can be greater than any of about 2, 4, 6, 8, 10, 12, 14, 16, 18, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 800, 825, 850, 875, 900, 925, 950, 975, 1000, 2000, 3000, 4000, 5000, 6000, 7000, 8000, 9000, 10,000 times tighter, for example as measured in a suitable activity assay such as for example an SPR (surface plasmon resonance) for example as described herein. Preferably the BMP receptor is a receptor of (i) BMP2, (ii) BMP2/6 heterodimer, (iii) BMP4, (iv) BMP5, (v) BMP6 or (vi) BMP7.

The inhibition or enhancement of BMP or a BMP polypeptide having BMP activity binding in-vitro to any one or more of (a) an agonist of BMP or BMP polypeptide having BMP activity, (b) an antagonist of BMP or BMP polypeptide having BMP activity, (c) a BMP receptor, (d) ERFE or ERFE polypeptide having erythroferrone activity can be measured by an in-vitro binding assay for BMP such as for example SPR (surface plasmon resonance) or HTRF (Homogeneous Time Resolved Fluorescence) assay as described herein. A homogenous time-resolved fluorescence assay (HTRF assay) can be used to identify agonists or antagonists of BMP such as anti-BMP, anti-BMP receptor or anti-ERFE antibodies or binding portions thereof that are capable of enhancing or inhibiting a BMP—partner molecule interaction. For example a recombinant BMP receptor labelled with europium cryptate is added to an assay mixture containing biotinylated human BMP and a dilution series of anti-BMP antibody is added and a fluorescence reading measured from which the IC50 may be calculated. The assay may be conducted at room temperature or 20° C., for example in a suitable assay buffer for example at room temperature or 20° C. Reactions can proceed for a period, for example 3 hours before taking data readings. Data can be obtained with excitation at 340 nm and two emission readings at 615 nm and 665 nm and readings can be expressed as a ratio of fluorescence at 665/615, optionally using an EnVision MultiLabel Plate Reader. Alternatively the ability of an anti-BMP antibody to inhibit binding of BMP to ERFE or ERFE polypeptide having erythroferrone activity can be determined using an SPR assay at room temperature or 20° C. for example run on the BIAcore T200. For example the ERFE or ERFE polypeptide having erythroferrone activity can be immobilized onto the flow cell, increasing concentrations of anti-BMP antibody are added in the presence of BMP and signal detected from which IC50 for inhibition of BMP-ERFE, or ERFE polypeptide having erythroferrone activity, interaction can be determined.

According to the present invention the BMP agonist or antagonist can be a small molecule agonist or antagonist. According to the present invention the BMP agonist or antagonist can be an agonist or antagonist immunoglobulin molecule, an agonist or antagonist antibody, capable of the specific and/or selective binding, such immunoglobulin or antibody can be an antibody or antigen binding fragment or portion thereof. The antibody or antigen binding portion thereof can specifically and/or selectively bind to and/or be raised against ERFE or an ERFE polypeptide having erythroferrone activity. The antibody or antigen binding portion thereof can specifically and/or selectively bind to and/or be raised against BMP or a BMP polypeptide having BMP activity, preferably of (i) BMP2, (ii) BMP2/6 heterodimer, (iii) BMP4, (iv) BMP5, (v) BMP6 or (vi) BMP7. The antibody or antigen binding portion thereof can specifically and/or selectively bind to and/or be raised against a BMP receptor, preferably a receptor of (i) BMP2, (ii) BMP2/6 heterodimer, (iii) BMP4, (iv) BMP5, (v) BMP6 or (vi) BMP7. In each case specific and/or selectively binding can be in-vitro and/or in-vivo.

According to the present invention the BMP agonist or antagonist can be a BMP receptor inhibitor for example, dorsomorphin, LDN-193189, LDN-212854, FMH1, K02288, LDN-213844, LDN-214117, a BMPR ligand trap. According to the present invention the BMP agonist or antagonist can be a BMP ligand for example, noggin, chordin, chordin-like 1, chordin-like 2, endoglin, Gremlin, Cerberus, follistatin, ectodin/uterine sensitization-associated gene-1 (USAG-1), and DAN family member. According to the present invention the BMP agonist or antagonist can be a E3 ubiquitine ligase such as Smurf1, Smurf2, or can be a transcriptional co-repressor such as c-Ski, SnoN, and Tob, or can be a feedback inhibitor such as BAMBI, SMAD6, SMAD7.

According to the present invention the BMP agonist or antagonist can be an antibody or antigen-binding portion thereof which binds to, specifically binds to, or selectively binds to ERFE or an ERFE polypeptide having erythroferrone activity preferably to (i) the N-terminal region of ERFE, or amino acid positions 1 to 190 or 1 to 212 of SEQ ID NO: 1 (ii) the SEQ ID NO: 3 (TNFD domain), or amino acid positions 190 to 354 of SEQ ID NO: 1, (iii) the SEQ ID NO: 4 (NTD2 domain), or amino acid positions 114 to 189 of SEQ ID NO: 1, (iv) the SEQ ID NO: 5 (Collagen Like Domain), or amino acid positions 96 to 113 of SEQ ID NO: 1, (v) the SEQ ID NO: 6 (NTD1 domain), or amino acid positions 24 to 95 of SEQ ID NO: 1, (v) the SEQ ID NO: 7 (SP domain), or amino acid positions 1 to 23 of SEQ ID NO: 1, (vi) a sequence consisting of amino acids 196 to 206 of SEQ ID NO:1, or the sequence set forth in SEQ ID NO: 8[GPRAPRVEAAF], (vii) a sequence consisting of amino acids 132 to 148 of SEQ ID NO:1, or the sequence set forth in SEQ ID NO: 9, [LLKEFQLLLKGAVRQRE], (viii) a sequence consisting of amino acids 109 to 125 of SEQ ID NO:1, or the sequence set forth in SEQ ID NO: 10, [GLPGPPGPPGPQGPPGP], (ix) a sequence consisting of amino acids 73 to 94 of SEQ ID NO:1, or the sequence set forth in SEQ ID NO: 11, [AHSVDPRDAWMLFVXQSDKGXN] or SEQ ID NO: 13 [AHSVDPRDAWMLFVRQSDKGVN], (x) a sequence consisting of amino acids 73 to 85 of SEQ ID NO:1, or the sequence set forth in SEQ ID NO: 12, [AHSVDPRDAWMLFV], (xi) a sequence consisting of or comprising all or part of the amino acid sequence [RDAWFVRQ], SEQ ID NO: 14, (xii) a sequence consisting of or comprising all or part of the amino acid sequence HSVDPRDAWM, SEQ ID NO:15, (xiii) a sequence consisting of or comprising all or part of the amino acid sequence DPRDAWFV, SEQ ID NO: 16, (xiv) a sequence consisting of or comprising all or part of the amino acid sequence DPRDAWMLFV, SEQ ID NO: 17, (xv) a sequence consisting of or comprising all or part of the amino acid sequences HSVDPRDAWM and RDAWFVRQ, SEQ ID NOs: 15 and 14, (xvi) a sequence consisting of or comprising all or part of the amino acid sequence SEQ ID NO:1 or sequence having 95 to 100% identity to SEQ ID NO: 1. According to the present invention the BMP agonist or antagonist can be an antibody or antigen-binding portion thereof which (i) specifically or selectively binds to a sequence consisting of or comprising all or part of the amino acid sequence RDAWFVRQ SEQ ID NO: 14, (ii) specifically or selectively binds to a sequence consisting of or comprising all or part of the amino acid sequence HSVDPRDAWM, SEQ ID NO: 15 (iii) specifically or selectively binds to a sequence consisting of or comprising all or part of the amino acid sequences HSVDPRDAWM and RDAWFVRQ, SEQ ID NOs: 15 and 14.

According to the present invention the BMP agonist or antagonist can be an antibody or antigen-binding portion thereof which comprises: (i) the CDR sequences: CDRH1, SEQ ID NO: 18, CDRH2, SEQ ID NO: 19, CDRH3, SEQ ID NO: 20, CDRL1, SEQ ID NO: 21, CDRL2, SEQ ID NO: 22, CDRL3, SEQ ID NO: 23, (ii) the Vh and Vl sequences, SEQ ID NO: 24, and SEQ ID NO: 25 respectively, or (iii) the heavy and light chain sequences, SEQ ID NO: 26, and SEQ ID NO: 37 respectively.

According to the present invention the BMP agonist or antagonist can be an immunoglobulin molecule agonist or antagonist capable of the specific and/or selective binding such as an antibody or antigen binding fragment or portion thereof. The antibody or antigen binding portion thereof can specifically and/or selectively bind to and/or be raised against ERFE or an ERFE polypeptide having erythroferrone activity or specifically and/or selectively bind to and/or be raised against BMP or a BMP polypeptide having BMP activity, or specifically and/or selectively bind to and/or be raised against a BMP receptor, as herein before described. According to the invention, the antibody, or an antigen-binding portion thereof, can specifically and/or selectively bind in-vitro and/or in-vivo. According to the invention the antibody or antigen binding portion thereof can be bi-specific and specifically and/or selectively bind to ERFE or an ERFE polypeptide having erythroferrone activity and/or specifically and selectively bind to BMP or a BMP polypeptide having BMP activity, preferably (i) BMP2, (ii) BMP2/6 heterodimer, (iii) BMP4, (iv) BMP5, (v) BMP6 or (vi) BMP7. According to the invention the antibody or antigen binding portion thereof can be bi-specific and specifically and/or selectively bind to a BMP receptor, preferably a receptor of (i) BMP2, (ii) BMP2/6 heterodimer, (iii) BMP4, (iv) BMP5, (v) BMP6 or (vi) BMP7, and specifically and selectively bind to BMP, preferably (i) BMP2, (ii) BMP2/6 heterodimer, (iii) BMP4, (iv) BMP5, (v) BMP6 or (vi) BMP7; and/or specifically and selectively bind to BMP or a BMP polypeptide having BMP activity, preferably (i) BMP2, (ii) BMP2/6 heterodimer, (iii) BMP4, (iv) BMP5, (v) BMP6 or (vi) BMP7.

The BMP agonist or antagonist, or the antibody or an antigen-binding portion thereof, can bind ERFE or an ERFE polypeptide having erythroferrone activity and/or bind BMP or a BMP polypeptide having BMP activity and/or bind BMP receptor in a dose or concentration dependent manner and/or can form a stable complex therewith. According to the invention, the BMP agonist or antagonist, or the antibody, or an antigen-binding portion thereof, can form a complex with ERFE or an ERFE polypeptide having erythroferrone activity and/or BMP or a BMP polypeptide having BMP activity and/or BMP receptor which can have a half life in-vitro and/or in-vivo and/or in biological fluid of about or more than any one of about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 hours+/−1 hour. The BMP agonist or antagonist, or the antibody or an antigen-binding portion thereof, can bind in a dose or concentration dependent manner to ERFE or an ERFE polypeptide having erythroferrone activity and/or BMP or a BMP polypeptide having BMP activity and/or BMP receptor and/or can form a stable complex therewith. According to an embodiment of the invention, the BMP agonist or antagonist or the antibody, or an antigen-binding portion thereof, can form a complex with ERFE or an ERFE polypeptide having erythroferrone activity and/or BMP or a BMP polypeptide having BMP activity and/or BMP receptor which can have a half life in-vitro and/or in-vivo and/or in biological fluid of about or more than any one of about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 days+/−1 day. In an embodiment, the half life is about or more than any one of about 5 days, 6 days, 20 days, 26 days, 27 days.

According to the foregoing invention the complex with the BMP agonist or antagonist, or the antibody, or an antigen-binding portion thereof, has a half life in-vivo or in biological fluid of about or more than 6 days. The stability in-vitro can be measured at about physiological pH, in a buffered aqueous solution, for example at 20° C. or 37° C., for example by SPR (surface Plasmon resonance, BIACORE), ELISA or radioimmunoassay to quantify the levels of active antibody by target binding or alternatively by determination of the soluble antibody level in solution using spectrophotometry. According to the foregoing embodiments, the in-vivo half life can be half life in a rat, mouse or human body or biological fluid thereof, for example human. The half life can also determined from serum or plasma measurements of the antibody-ERFE complex levels following introduction of the antibody into a biological fluid sample or its administration in-vivo for example by intravenous or subcutaneous injection.

According to the invention the complex of the BMP agonist or antagonist, or the antibody or an antigen-binding portion thereof has a prolonged half life, higher stability in-vivo for example in serum is desirable as it permits a dosage regime of less frequent dosing and/or lower dosing levels hence reducing risk of any potential toxicity or side effects in-vivo. High stability of the BMP agonist or antagonist or the antibody, or an antigen-binding portion thereof, complex is an indicator of higher potency and has the mentioned benefit that the antibody can be used at lower dosage amounts than a less specific and/or less selective and/or less potent BMP agonist, antagonist or antibody to achieve the same therapeutic efficacy hence reducing potential toxicity or side effects in-vivo.

The BMP agonist or antagonist, or the antibody, or antigen-binding portion thereof, or a complex therewith, can have a half life in-vivo of about or more than any one of about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 40, 42, 44, 426, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, or 600 hours+/−1 hour. For example the BMP antagonist or agonist, or the antibody, or antigen-binding portion thereof, or complex therewith, can have a half life in-vivo of between about 163 and 540 hours and/or about or more than about 163 hours. The BMP agonist or antagonist, or the antibody, or antigen-binding portion thereof, or a complex therewith, can have a half life in-vivo of about or more than any one of about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 days+/−1 day, for example the BMP agonist or antagonist, or the antibody, or antigen-binding portion thereof, or a complex therewith, has a half life in-vivo of between about 6 and 22 days, for example of about or more than about 6 days.

According to the foregoing embodiments, the in-vivo half life can be the half life in rat, mouse or human body or biological fluid thereof. The half life can be determined from plasma or serum measurements of the levels of the BMP agonist or antagonist, or the antibody, or antigen-binding portion thereof, or a complex therewith following administration in-vivo for example by intravenous or subcutaneous injection.

According to an embodiment of the present invention, the antibody or an antigen-binding portion thereof, can be human, humanised or chimeric.

The antibody or an antigen-binding portion thereof can have an isotype subclass selected from the group consisting of IgG1, of IgG₂, IgG₄, IgG_{2Δa}, IgG_{4Δb}, IgG_{4Δc}, IgG₄ S228P, IgG_{4Δb} S228P and IgG_{4Δc} S228P. The antibody or an antigen-binding portion thereof, can be a full length-antibody of an IgG1, of IgG₂, IgG₄, IgG_{2Δa}, IgG_{4Δb}, IgG_{4Δc}, IgG₄ S228P, IgG_{4Δb} S228P or IgG_{4Δc} S228P isotype. The antibody or an antigen-binding portion thereof, may be a single chain antibody, a Fab fragment, a F(ab)₂ fragment, a Fv fragment. The antibody or an antigen-binding portion thereof, may be a tetrameric antibody, a tetravalent antibody, a bi-specific or multispecific antibody, a domain-specific antibody, a single domain antibody. The antibody or an antigen-binding portion thereof, may be a fusion protein. The invention also provides a bispecific molecule comprising the antibody, or antigen-binding portion thereof, of the invention, linked to a second functional moiety having a different binding specificity than said antibody, or antigen binding portion thereof.

According to the present invention the BMP agonist or antagonist can be ERFE or an ERFE polypeptide having erythroferrone activity and/or which binds to BMP, preferably to (i) BMP2, (ii) BMP2/6 heterodimer, (iii) BMP4, (iv) BMP5, (v) BMP6 or (vi) BMP7; further preferably to BMP2 and/or BMP4, or BMP5 and/or BMP6 and/or BMP7. Accordingly the BMP agonist or antagonist can be (i) ERFE of SEQ ID NO:1 or sequence having 95 to 100% identity to SEQ ID NO: 1 or can be (ii) an isolated ERFE polypeptide consisting of an N-terminal region of EFRE comprising a C-terminal truncation of amino acid sequence SEQ ID NO:1 or sequence having 95 to 100% identity to SEQ ID NO: 1.

Accordingly the C-terminal truncation can be within the TNF like domain, wherein the TNF like domain comprises amino acids 190 to 354 of amino acid sequence SEQ ID NO:1 or sequence having 95 to 100% identity to SEQ ID NO: 1, optionally wherein the TNF like domain is truncated between amino acids 190 and 212, preferably 212 or wherein the TNF like domain is deleted.

Alternatively the C-terminal truncation can be within the NTD2 domain, wherein the NTD2 domain comprises amino acids 114 to 189 of amino acid sequence SEQ ID NO:1 or sequence having 95 to 100% identity to SEQ ID NO: 1, optionally wherein the C-terminal truncation is at amino acid position 142 or wherein the NTD2 domain is deleted.

Alternatively the C-terminal truncation can be within the collagen-like domain, wherein the collagen-like domain comprises amino acids 96 to 113 of amino acid sequence SEQ ID NO:1 or sequence having 95 to 100% identity to SEQ ID NO: 1, optionally wherein the C-terminal truncation is at amino acid position 96 or 112 or wherein the collagen domain is deleted.

Alternatively the C-terminal truncation can be within the NTD1 domain, wherein the NTD1 domain comprises amino acids 24 to 95 of amino acid sequence SEQ ID NO:1 or sequence having 95 to 100% identity to SEQ ID NO: 1, optionally wherein the NTD1 domain is truncated at amino acid position 42.

The ERFE or an ERFE polypeptide having erythroferrone activity can comprise or consist of (i) a sequence consisting of amino acids 196 to 206 of SEQ ID NO:1, or the sequence set forth in SEQ ID NO: 8 [GPRAPRVEAAF, SEQ ID NO: 8]; (ii) a sequence consisting of amino acids 132 to 148 of SEQ ID NO:1, or the sequence set forth in SEQ ID NO: 9, [LLKEFQLLLKGAVRQRE, SEQ ID NO: 9]; (iii) a sequence consisting of amino acids 109 to 125 of SEQ ID NO:1, or the sequence set forth in SEQ ID NO: 10, [GLPGPPGPPGPQGPPGP, SEQ ID NO: 10]; (iv) a sequence consisting of amino acids 73 to 94 of SEQ ID NO:1, or the sequence set forth in SEQ ID NO: 11, [AHSVDPRDAWMLFVXQSDKGXN, SEQ ID NO: 11]; or (v) a sequence consisting of amino acids 73 to 85 of SEQ ID NO:1, or the sequence set forth in SEQ ID NO: 12, [AHSVDPRDAWMLFV, SEQ ID NO: 12].

The ERFE or an ERFE polypeptide having erythroferrone activity can lack an SP domain, wherein the SP domain comprises amino acids 1 to 24 of amino acid sequence SEQ ID NO:1 or sequence having 95 to 100% identity to SEQ ID NO: 1. Preferably the ERFE or an ERFE polypeptide having erythroferrone activity exhibits erythroferrone activity which is similar or the same as the erythroferrone activity exhibited by EFRE of SEQ ID NO:1. Preferably the ERFE or an ERFE polypeptide having erythroferrone activity decreases and/or inhibits hepcidin activity, hepcidin expression or hepcidin mRNA production, inhibits BMP activity, binds BMP or BMP polypeptide having BMP activity, preferably (i) BMP2, (ii) BMP2/6 heterodimer, (iii) BMP4, (iv) BMP5, (v) BMP6 or (vi) BMP7; further preferably to BMP2 and/or BMP4, or BMP5 and/or BMP6 and/or BMP7. In the foregoing description the term having “95 to 100% identity to SEQ ID NO: 1” may be read to include “having 95%, 96%, 97%, 98%, 99%, or 100% identity to SEQ ID NO: 1 or such identity over the equivalent length of polypeptide sequence in SEQ ID NO:1”.

Nucleic Acids, Vectors, Cells

According to a third aspect of the present invention there is provided (i) a method of treating, preventing, ameliorating, controlling, reducing incidence of, or delaying the development or progression of the development or progression of a disease of iron metabolism or (ii) a method of treating, preventing, ameliorating, controlling, reducing incidence of, or delaying the development or progression of the development or progression of a disease of lipid or carbohydrate metabolism using the nucleic acid encoding the BMP agonist or antagonist or vector comprising the nucleic acid such as a gene delivery vector, for example an AAV vector. The vector can be a replicable expression vector, optionally for transfecting a mammalian cell, for example the vector can be a viral vector for example an AAV vector.

According to the present invention as herein described the BMP agonist or antagonist can be: (1.1) ERFE or an ERFE polypeptide having erythroferrone activity, (1.2) BMP or a BMP polypeptide having BMP activity; preferably BMP2, BMP2/6 heterodimer, BMP4, BMP5, BMP6 or BMP7, (1.3) a BMP receptor or fusion protein thereof, (1.4) an antibody or antigen binding portion thereof which can specifically and/or selectively bind to and/or is raised against: (i) ERFE or an ERFE polypeptide having erythroferrone activity, (ii) BMP or a BMP polypeptide having BMP activity; preferably BMP2, BMP2/6 heterodimer, BMP4, BMP5, BMP6 or BMP7, (iii) a BMP receptor, preferably receptor of BMP2, BMP2/6 heterodimer, BMP4, BMP5, BMP6 or BMP7. According to the present invention as herein described the BMP agonist or antagonist can be a nucleic acid encoding the BMP agonist or antagonist recited herein for example encoding any of (1.1)-(1.4) above or vector comprising the nucleic acid such as a gene delivery vector, for example an AAV vector.

The present invention therefore provides nucleic acids encoding the BMP agonist or antagonist according to the invention and vectors and cells comprising such nucleic acids as well as methods of producing the BMP agonist or antagonist from the cells for example by expression from the cells and optional subsequent purification. The invention further provides a nucleic acid molecule encoding the BMP agonist or antagonist and/or complementary nucleic acid thereof. According to the present invention the nucleic acid molecule may further comprise a region encoding a signal sequence, for example a DNA or RNA sequence or for example an immunoglobulin signal sequence. The invention further provides a replicable expression vector for transfecting a cell, the vector comprising the nucleic acid molecule of the invention. In an embodiment, the vector is a viral vector. The vector can be for use as a medicament and/or for use in the prevention and/or treatment of a disorder of iron metabolism and/or disease or disorder comprising abnormally low or high iron levels and/or a disease or disorder comprising abnormally low or high hepcidin levels and/or symptoms thereof in an individual, and/or a disease of carbohydrate or lipid metabolism.

The invention further provides a method of expressing the nucleic acid molecule or the vector of the invention to produce or secrete the BMP agonist or antagonist according to the invention. The method can comprise the introduction of the nucleic acid molecule or vector into a cell and expression of the nucleic acid therein to produce or secrete the BMP agonist or antagonist according to the invention. The nucleic acid molecule or vector can be introduced into the cell in-vitro alternatively in-vivo. The expressed BMP agonist or antagonist, can be expressed in-vitro, optionally further isolated and purified. The expressed BMP agonist or antagonist, can be expressed in-vivo, the in-vivo expression such that it can constitute gene therapy. The vector can be a replicable expression vector, optionally for transfecting a mammalian cell, for example the vector can be a viral vector for example an AAV vector.

The invention further provides a host cell harbouring the nucleic acid molecule or vector of either the third or fourth aspect, for example the cell can be a eukaryotic cell or a prokaryotic cell, for example a bacterial cell a yeast cell or a mammalian cell. In an embodiment, the host cell is a mammalian cell.

Pharmaceutical Compositions

According to a fourth aspect of the present invention there is provided a method of treating, preventing, ameliorating, controlling, reducing incidence of, or delaying the development or progression of the development or progression of a disease of iron metabolism or a disease of lipid or carbohydrate metabolism, using a pharmaceutical composition comprising the BMP agonist or antagonist or nucleic acid encoding the BMP agonist or antagonist or vector comprising the nucleic acid according to any of the foregoing aspects of the invention further comprising a pharmaceutically acceptable carrier and/or an excipient. According to one embodiment the pharmaceutical composition can comprise one or more BMP agonists or antagonists according to the invention and/or one or more nucleic acids encoding the BMP agonist or antagonist or vectors comprising the nucleic acid.

Combination Therapy

According to a fifth aspect of the present invention there is provided in one embodiment a method of treating a disease of iron metabolism using a BMP agonist or antagonist, or nucleic acid encoding the BMP agonist or antagonist or vector comprising the nucleic acid, or pharmaceutical composition thereof, according to the first, third and fourth aspects, wherein the BMP agonist or antagonist or nucleic acid encoding the BMP agonist or antagonist or vector comprising the nucleic acid or pharmaceutical composition is provided for use separately, sequentially or simultaneously in combination with a second therapeutic agent, optionally wherein the combination is provided as a pharmaceutical composition comprising a pharmaceutically acceptable carrier and/or an excipient. The second therapeutic agent may be selected from a BMP agonist or antagonist, an agonist such as for example any one or more BMP agonist or antagonist or nucleic acid encoding the BMP agonist or antagonist or vector comprising the nucleic acid or pharmaceutical composition already herein before described. The second therapeutic agent may be selected from: red blood cells for example as provided by transfusion or erythocytapheresis, iron chelators, such as for example deferoxamine or deferiprone, folate. The second therapeutic agent may be selected from one or more of: hydroxyurea, hypomethylating agents, histone deacetylase inhibitors, erythropoietin, antioxidants such as for example: vitamin E, acetylcysteine, deferiprone; bone or bone marrow stem cells, for example as provided by allogeneic transplantation, thalidomide, lenalidomide, sirolimus, ruxolitinib, pacritinib, a JAK2 inhibitor, luspatercept, sotatercept, a mini-hepcidin, apo-transferrin, β- or γ-globin for example as provided by gene addition, a regulator of globinsynthesis.

According to a fifth aspect of the present invention there is provided in one embodiment a method of treating a disease of lipid or carbohydrate metabolism using a BMP agonist or antagonist, or nucleic acid encoding the BMP agonist or antagonist or vector comprising the nucleic acid, or pharmaceutical composition thereof, according to the second, third and fourth aspects, wherein the BMP agonist or antagonist or nucleic acid encoding the BMP agonist or antagonist or vector comprising the nucleic acid or pharmaceutical composition is provided for use separately, sequentially or simultaneously in combination with a second therapeutic agent as herein described according to the foregoing aspects, optionally wherein the combination is provided as a pharmaceutical composition comprising a pharmaceutically acceptable carrier and/or an excipient. The second therapeutic agent may be selected from a BMP agonist or antagonist or nucleic acid encoding the BMP agonist or antagonist or vector comprising the nucleic acid or pharmaceutical composition already herein before described. The second therapeutic agent may be selected from one or more of insulin sensitizers, metformin, thiazolidinedione, statins, pentoxifylline, diuretics, ACE inhibitors, simvastatin, sitagliptin, GLP-1 agonists, insulin, or synthetic insulin analogs.

Methods of Treatment

According to the first, third, fourth and fifth aspects of the present invention there is further provided a method of treating, preventing, ameliorating, controlling, reducing incidence of, or delaying the development or progression of the development or progression of a disease of iron metabolism using a BMP agonist or antagonist, nucleic acid, nucleic acid complement, vector or pharmaceutical composition thereof, or combination according to the foregoing aspects, wherein the degree to which the concentration or level of a biomarker of the disease of iron metabolism deviates from normal concentration or level is reduced by the use or administration of the BMP agonist or antagonist, nucleic acid, nucleic acid complement, vector or pharmaceutical composition thereof, or combination. The normal or control concentration or control or level of biomarker can be judged from a control sample, for example from an individual not having the disease of iron metabolism, or can be the normal or control concentration or level of biomarker in an individual not having the disease iron metabolism as known from, published or accepted in the art. The control can be an individual of equivalent gender, age, such as adult or child, or sample therefrom. The use or administration of the BMP agonist or antagonist, nucleic acid, nucleic acid complement, vector or pharmaceutical composition thereof, or combination can reduce the deviation of the biomarker level or concentration from normal or control biomarker level or concentration by about or more than 5 percent, 10 percent, 15 percent, 20 percent, 25 percent, 30 percent, 35 percent, 40 percent, 45 percent, 50 percent, 55 percent, 60 percent, 65 percent, 70 percent, 75 percent, 80 percent, 85 percent, 90 percent or 95 percent or greater, for example, 96 percent, 97 percent, 98 percent, 99 percent or 100 percent. The use or administration of the BMP agonist or antagonist, nucleic acid, nucleic acid complement, vector or pharmaceutical composition thereof, or combination according to the foregoing aspects can reduce the deviation of the biomarker level or concentration from normal or control biomarker level or concentration within a period of or less than 2 hours, 4 hours, 6 hours, 8 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days.

A biomarker of disease of iron metabolism can be blood or serum hepcidin, serum ferritin or transferrin or iron accumulation in liver, hepatic iron index (HII), total iron binding capacity, mean corpuscular volume (MCV), mean corpuscular hemoglobin (MCH), mean corpuscular hemoglobin concentration (MCHC), hematocrit or packed cell volume (PCV), total red blood cell (RBC) count or blood hemoglobin, for example low level of RBC or hemoglobin is a sign of anemia.

According to the second, third, fourth and fifth aspects of the present invention there is further provided a method of treating, preventing, ameliorating, controlling, reducing incidence of, or delaying the development or progression of the development or progression of a disease of lipid or carbohydrate metabolism using a BMP agonist or antagonist, nucleic acid, nucleic acid complement, vector or pharmaceutical composition thereof, or combination, wherein the degree to which the concentration or level of a biomarker of the disease of lipid or carbohydrate metabolism deviates from normal or control concentration or level is reduced by the use or administration of the BMP agonist or antagonist, nucleic acid, nucleic acid complement, vector or pharmaceutical composition thereof, or combination. The normal or control concentration or level of biomarker can be judged from a control sample, for example from an individual not having the disease of lipid or carbohydrate metabolism, or can be the normal or control concentration or level of biomarker in an individual not having the disease of lipid or carbohydrate metabolism as known from, published or accepted in the art. The control can be an individual of equivalent gender, age, such as adult or child, or sample therefrom. The use or administration of the BMP agonist or antagonist, nucleic acid, nucleic acid complement, vector or pharmaceutical composition thereof, or combination can reduce the deviation of the biomarker level or concentration from normal or control biomarker level or concentration by about or more than 5 percent, 10 percent, 15 percent, 20 percent, 25 percent, 30 percent, 35 percent, 40 percent, 45 percent, 50 percent, 55 percent, 60 percent, 65 percent, 70 percent, 75 percent, 80 percent, 85 percent, 90 percent or 95 percent or greater, for example, 96 percent, 97 percent, 98 percent, 99 percent or 100 percent. The use or administration of the BMP agonist or antagonist, nucleic acid, nucleic acid complement, vector or pharmaceutical composition thereof, or combination according to the foregoing aspects can reduce the deviation of the biomarker level or concentration from normal or control biomarker level or concentration within a period of or less than 2 hours, 4 hours, 6 hours, 8 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days. A biomarker of disease of lipid or carbohydrate metabolism can be platelet count, mean platelet volume (MPV), blood insulin, blood sugar, serum triglycerides, blood high-density lipoprotein (HDL) level, blood pressure, blood cholesterol, serum level of hyaluronic acid, cytokeratin-18 (CK-18) and Collagen 7s. A biomarker can further include serum concentration of CRP, for example as detected by hs-CRP testing and which is elevated in NAFLD, obesity, insulin resistance and metabolic syndrome. Further biomarkers can include blood or serum ferritin or transferrin or iron accumulation in liver or fat accumulation levels in hepatic cells, these are increased for example in patients with NAFLD, steatohepatitis and NASH. A biomarker can additionally be serum albumin, serum malondialdehyde, serum plasma pentraxin 3, blood transaminases of alanine aminotransferase (ALT) and blood aspartate aminotransferase (AST), blood alkaline phosphatase (ALKP), additionally also serum leptin, serum adipokines, serum adipocytokines, serum adiponectin, levels of which are for example altered in metabolic syndrome, insulin resistance, NASH and NAFLD. Further biomarkers can include blood or serum level of IL-6 or of TNF-α and its soluble receptors, insulin resistance for example as measured by the metabolic clearance rate of glucose, these are significantly higher for example in NAFLD, insulin resistance and obesity. A biomarker can further include, body mass index, total body fat or distribution of adipose tissue, central-to-peripheral fat distribution gluteal to abdominal fat distribution for example as measured by dual energy x ray absorptiometry (DEXA) and imaging techniques.

According to the second, third, fourth and fifth aspects of the present invention the use or administration of the BMP agonist or antagonist, nucleic acid, nucleic acid complement, vector or pharmaceutical composition thereof, or combination according to the foregoing aspects can achieve an improvement in a diagnostic test or diagnostic test score for a disease of lipid or carbohydrate metabolism, for example compared to the test or score prior to use or administration or in comparison to an untreated individual or sample therefrom. The diagnostic test or diagnostic test score can be for example the BAAT score [Ratziu V, et. al., Gastroenterology. 2000; 118:1117-1123], F1B4 index [Sumida Y, et. al, BMC Gastroenterol. 2012; 12:2], FibroTest/NASH test [Ratziu V, et. al., Aliment Pharmacol Ther. 2007; 25:207-218], FibroMeter/NAFLD Fibrosis Score or NFS test [Calès P, et. al., Liver Int. 2010; 30:1346-1354.], AST to ALT ratio and the AST to platelet ratio index (APRI).

Treatment Regimen and Dosage

The BMP agonist or antagonist, according to the first or second aspects, or the nucleic acid molecule or vector according to the third aspect, the pharmaceutical composition according to the fourth aspect or the combination according to the fifth aspect can be prepared for or be suitable for oral, sublingual, buccal, topical, rectal, inhalation, transdermal, subcutaneous, intravenous, intra-arterial, intramuscular, intracardiac, intraosseous, intradermal, intraperitoneal, transmucosal, vaginal, intravitreal, intra-articular, peri-articular, local or epicutaneous administration, which can be prior to and/or during and/or after the onset of the aforementioned conditions for therapy or for such use.

Preferably the BMP agonist or antagonist, according to the first or second aspects, or the nucleic acid molecule or vector according to the third aspect, the pharmaceutical composition according to the fourth aspect or the combination according to the fifth aspect is for, or is prepared for, administration between once to 7 times per week, for example around once twice, three, four, five six or seven times per week, by further example between once to four times per month, or between once to six times per 6 month period, or once to twelve times per year. Additionally preferably is prepared to be, peripherally administered in a period selected from: once daily, once every two, three, four, five or six days, weekly, once every two weeks, once every three weeks, monthly, once every two months, once every three months, once every four months, once every five months, once every six months, once every seven months, once every eight months, once every nine months, once every ten months, once every eleven months or yearly.

Preferably the BMP agonist or antagonist, according to the first or second aspects, or the nucleic acid molecule or vector according to the third aspect, the pharmaceutical composition according to the fourth aspect or the combination according to the fifth aspect can be, is, or is prepared to be, peripherally administered via a route selected from one or more of; orally, sublingually, buccally, topically, rectally, via inhalation, transdermally, subcutaneously, intravenously, intra-arterially or intramuscularly, via intracardiac administration, intraosseously, intradermally, intraperitoneally, transmucosally, vaginally, intravitreally, epicutaneously, intra-articularly, intravesically, intrathecally, peri-articularly or locally. In an embodiment the administration is intravenous or subcutaneous administration.

Preferably the BMP agonist or antagonist, according to the first or second aspects, or the nucleic acid molecule or vector according to the third aspect, the pharmaceutical composition according to the fourth aspect or the combination according to the fifth aspect is for, or is prepared for, administration at a concentration of between about 0.1 to about 200 mg/ml; for example at any one of about 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190 or 200 mg/ml+/−about 10% error, for example at about 50 mg/ml, for example with respect to the respective active ingredient.

Preferably the BMP agonist or antagonist, according to the first or second aspects or the embodiments thereof, or the nucleic acid molecule or vector according to the third aspect, the pharmaceutical composition according to the fourth aspect or the combination according to the fifth aspect is for, or is prepared for, administration at a concentration of between about 0.01 to about 200 mg/kg of body weight; for example at any one of about 0.1, 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190 or about 200 mg/kg of body weight+/−about 10% error, for example at about 10 mg/kg, for example with respect to the respective active ingredient.

The BMP agonist or antagonist, according to the first or second aspects, or the nucleic acid molecule or vector according to the third aspect, the pharmaceutical composition according to the fourth aspect or the combination according to the fifth aspect can be administered to an individual via any suitable route. It should be apparent to a person skilled in the art that the examples described herein are not intended to be limiting but to be illustrative of the techniques available. Accordingly administration may be in accordance with known methods, such as intravenous administration, e.g., as a bolus or by continuous infusion over a period of time, by intramuscular, intraperitoneal, intracerebrospinal, transdermal, subcutaneous, intraarticular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, the BMP agonist or antagonist, nucleic acid molecule, vector, pharmaceutical composition or combination according to the foregoing aspects can be aerosolized using a fluorocarbon formulation and a metered dose inhaler, or inhaled as a lyophilized and milled powder. Administration can be site-specific or targeted local delivery including via various implantable depot sources of the medicament or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of a BMP agonist or antagonist, nucleic acid, vector, pharmaceutical composition or combination according to the foregoing aspects may be used for administration. In some embodiments, these may be administered neat, alternatively comprising a pharmaceutically acceptable excipient. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000. In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

The BMP agonist or antagonist, according to the first or second aspects or the embodiments thereof, or the nucleic acid molecule or vector according to the third aspect, the pharmaceutical composition according to the fourth aspect or the combination according to the fifth aspect can be administered using any suitable method, including by injection (e.g., intraperitoneally, intravenously, subcutaneously, intramuscularly, etc.). These can also be administered topically or via inhalation, as described herein. Generally, for administration, an initial candidate dosage can be about 2 mg/kg. For the purpose of the present invention, a typical daily dosage might range from about any of 3 mg/kg to 10 mg/kg, 3 mg/kg to 30 mg/kg, 3 mg/kg, to 100 mg/kg, 3 mg/kg, to 300 mg/kg or more, depending on the factors mentioned above. For example, dosage of about 1 mg/kg, about 2.5 mg/kg, about 5 mg/kg, about 10 mg/kg, and about 25 mg/kg may be used. For repeated administrations over several days or longer, depending on the condition, the treatment is sustained until a desired suppression of symptoms or conditions occur, until sufficient therapeutic levels are achieved, or until the aforementioned deviation of a biomarker level or concentration from normal or control biomarker level or concentration is reduced for example, to reduce, prevent or treat the relevant disease or condition. The progress of this therapy is easily monitored by conventional techniques and assays and the dosing regimen can vary over time.

For the purpose of the present invention, the appropriate dosage of the BMP agonist or antagonist, nucleic acid molecule, vector, pharmaceutical composition or combination employed, will depend on the type and severity of the disease of iron metabolism or lipid or carbohydrate metabolism to be treated, whether the agent is administered for preventive or therapeutic purposes, whether there has been previous therapy, the patient's clinical history and response to the agent or agents used, the clearance rate for the administered agent, and the discretion of the attending physician. Typically the clinician will administer the BMP agonist or antagonist, nucleic acid molecule, vector, pharmaceutical composition or combination until a dosage is reached that achieves the desired result of treating the disease. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on prevention and/or treatment and/or suppression and/or amelioration and/or delay of the disease of iron metabolism or lipid or carbohydrate metabolism. Alternatively, sustained continuous release formulations of BMP agonist or antagonist, nucleic acid molecule, vector, pharmaceutical composition or combination may be appropriate. Various formulations and devices for achieving sustained release are known in the art.

In one embodiment, dosages for a BMP agonist or antagonist according to the foregoing aspects may be determined empirically in individuals who have been given one or more administration(s) of the BMP agonist or antagonist, nucleic acid molecule, vector, pharmaceutical composition or combination, optionally wherein assessment of efficacy is by monitoring an indicator of the disease such as any of the aforementioned biomarkers. Alternatively, administration can be continuous or intermittent, depending, for example, upon the recipient's physiological condition, whether the purpose of the administration is therapeutic or prophylactic, and other factors known to skilled practitioners. For example, administration may be essentially continuous over a preselected period of time or may be in a series of spaced doses.

The treatment provided according to the present invention is for treatment of an individual for example the individual is a human, or a companion animal such as a horse, cat or dog or a farm animal such as a sheep, cow or pig; preferably a human.

Therapeutic Formulations

Therapeutic formulations of the BMP agonist or antagonist, nucleic acid molecule, vector, pharmaceutical composition or combination according to any of the preceding aspects of the invention can be are prepared for storage by mixing at the desired degree of purity with optional pharmaceutically acceptable carriers, excipients or stabilizers (Remington, The Science and Practice of Pharmacy 20th Ed), Mack Publishing, 2000), in the form of lyophilized formulations or aqueous solutions.

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the BMP agonist or antagonist, nucleic acid molecule, vector, pharmaceutical composition or combination can be prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations for use in in-vivo administration must be sterile. This is readily accomplished by, for example, filtration through sterile filtration membranes. The therapeutic compositions according to the aforementioned aspects are generally placed into a container having a sterile access port, for example, an intravenous solution bag or vial having a stopper pierceable by a hypodermic injection needle.

The compositions according to the present invention may be in unit dosage forms such as solid compositions, tablets, pills, capsules, powders, granules, solutions or suspensions, or suppositories, for oral, parenteral or rectal administration, or administration by inhalation or insufflation.

Kits

According to a further aspect of the present invention there is provided a kit comprising:

(a) the BMP agonist or antagonist, nucleic acid, nucleic acid complement, vector or pharmaceutical composition thereof, or combination, according to any of the preceding aspects; and

(b) instructions for the administration of an effective amount of said BMP agonist or antagonist, nucleic acid, nucleic acid complement, vector or pharmaceutical composition thereof, or combination, to an individual for treating, preventing, ameliorating, controlling, reducing incidence of, or delaying the development or progression of the development or progression of a disease of iron metabolism or a disease of lipid or carbohydrate metabolism or symptoms thereof.

The kit may include one or more containers containing the a BMP agonist or antagonist, nucleic acid, nucleic acid complement, vector or pharmaceutical composition thereof, or combination described herein and instructions for use in accordance with any of the methods and uses of the invention. The kit may further comprise a description of selecting an individual suitable for treatment based on identifying whether that individual has a disease of iron metabolism or a disease of carbohydrate or lipid metabolism or symptom thereof or is at risk of having such disease. The instructions for the administration of the pharmaceutical composition may include information as to dosage, dosing schedule and routes of administration for the intended treatment.

Generally, kit instructions comprise a description of administration of the BMP agonist or antagonist, nucleic acid, nucleic acid complement, vector or pharmaceutical composition thereof, or combination, for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of a BMP agonist or antagonist, nucleic acid, nucleic acid complement, vector or pharmaceutical composition thereof, or combination, generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar™ or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a BMP agonist or antagonist, nucleic acid, nucleic acid complement, vector. The container may further comprise a second pharmaceutically active agent.

Kits may optionally provide additional components such as buffers and interpretive information. Normally, the kit comprises a container and a label or package insert(s) on or associated with the container.

The invention also provides diagnostic kits comprising an antibodies specifically binding a biomarker as described herein in a sample. In some embodiments, a diagnostic kit can be used to identify an individual at risk of developing a disease of iron metabolism or disease or lipid or carbohydrate metabolism.

Diagnostic kits of the invention include one or more containers comprising an anti-biomarker antibody specifically binding a biomarker described herein and instructions for use in accordance with any of the methods of the invention described herein. Generally, these instructions comprise a description of use of the anti-biomarker antibody to detect the presence of a biomarker in individuals at risk of developing a disease of iron metabolism or disease or lipid or carbohydrate metabolism. In some embodiments, an exemplary diagnostic kit can be configured to contain reagents such as, for example, an anti-biomarker antibody, a negative control sample, a positive control sample, and directions for using the kit.

### DESCRIPTION OF FIGURES

FIG. 1A: Effect of BMPs of hepcidin-nanoluciferase fusion expression: The indicated example BMPs (BMP2, 6, 9) were added to NanoLuc cells, a dose-dependent increase on hepcidin expression is observed, EC50 shown in pM.

FIG. 1B: A schematic representation of the “nano-luc” nanoluciferase reporter construct.

FIG. 2: ERFE binds to BMP2, BMP4 and BMP6 with different affinities as measured by SPR/Biacore™.

FIG. 3A-F. BMP/SMAD signalling is suppressed by ERFE:

FIG. 3A. Gene expression analysis (Illumina) of Huh7 cells treated with human or mouse ERFE (10 μg/ml) for 24 h. Values represent Log(fold change) of genes differentially expressed in cells treated with human or mouse ERFE.

FIG. 3B. Gene expression measured by qRT-PCR of selected BMP/SMAD target genes and FGA in Huh7 cells treated with vehicle or mouse ERFE (10 μg/ml).

FIG. 3C. Huh7 cells treated with mouse ERFE (10 μg/ml), BMP6 and LDN (100 nM), alone or in combination, for 30 min. pSMAD/SMAD ratios values were calculated by densitometry from Western blot.

FIG. 3D. Huh7 cells treated with mouse ERFE (10 μg/ml), BMP6 and LDN (100 nM), alone or in combination, for 30 min, western blot.

FIG. 3E. C2C12 Bre-Luc cells were treated with 2 nM of BMP in combination with a gradient of mouse ERFE concentrations (7.5 pM to 0.5 μM) for 24 h, and luminescence measured in each well. Data was normalized to percentage of maximum luminescence (no ERFE)

FIG. 3F. Huh7 cells were treated with 2 nM of BMPs, alone or in combination with 10 μg/ml of mouse ERFE, in serum-free media, and analysed 6 h after treatment. Gene expression of HAMP and ID1 was measured by qRT-PCR. Results represented as average+/−standard deviation from three independent experiments (*p<0.05, **p<0.01, ***p<0.001, ****p<0.0001, Student's t test).

FIGS. 3G and 3H: ERFE suppresses BMP/SMAD signalling by inhibiting BMP 2/6, BMP6 and BMP7 in Huh7 cells: 2 nM of BMPs+/−10 μg/ml of mouse ERFE, 6 hr incubation in serum-free media, gene expression of HAMP and ID1 measured by qRT-PCR, results expressed as fold change relative to non-treated cells from 3 independent experiments, statistical significance analysed for each pair of BMP treatments. (*p<0.05, **p<0.01, ***p<0.001, ****p<0.0001, Student's t test).

FIGS. 3J and 3K: ERFE suppresses BMP/SMAD signalling by inhibiting BMP 2/6, BMP6 and BMP7 in HepG2 cells: 2 nM of BMPs+/−10 μg/mlof mouse ERFE, 6 hr incubation in serum-free media, gene expression of HAMP and ID1 measured by qRT-PCR, results expressed as fold change relative to non-treated cells from 3 independent experiments, statistical significance analysed for each pair of BMP treatments. (*p<0.05, **p<0.01, ***p<0.001, ****p<0.0001, Student's t test).

FIG. 3L: monoFC-huErfe suppresses hepcidin production in a dose-dependent fashion: HepG2 cells containing a stably integrated Nanoluciferase reporter construct, were treated with monoFC-huErfe A4 mutant at the indicated concentrations for 24 hours, prior to harvesting of supernatant and measurement of fluorescence monoFC

FIG. 4: EPO suppresses BMP-target genes in an ERFE-dependent manner in-vivo: (A,B) WT and ERFE KO male mice (10-13 weeks old) were injected with 3 doses of 200 u of EPO, one dose every 24 h, and analysed 24 h after the last injection to measure expression of BMP-target genes in the liver measured by qRT-PCR; and serum and liver iron analysis; (*p<0.05, **p<0.01, ***p<0.001, ****p<0.0001, using two-way ANOVA, n=6-8 mice per group).

FIG. 5: ERFE suppresses BMP-target genes in vivo: Nine weeks old WT male mice were injected i.v. with 200 μg of the monoFC-muErfe or a mono-Fc control that contained the N-terminal 14 amino acids of human ERFE. Mice were analysed 3 h after the injections to measure serum and liver iron and expression of BMP-target genes in the liver, (*p<0.05, **p<0.01, ***p<0.001, ****p<0.0001, using Student's t test, n=6-8 mice per group).

FIG. 6. The C1q domain is not required for erythroferrone activity: (FIG. 6A) Huh7 cells were treated for 24 h with full-length or C1q domain of human erythroferrone (10 μg/ml). (FIG. 6B) Huh7 cells were treated for 24 h with adipoferrone (construct containing N-terminal domain of adiponectin and C1q domain of erythroferrone) or C1q trimer (Tri-C1q) derived from mouse erythroferrone at 10 μg/ml. Gene expression measured by qRT-PCR. Data represents mean+standard deviation (n=3). ***p<0.001; ****p<0.0001.

FIG. 7. Mutation of the RARR furin cleavage site at the N-terminal of Erfe prevents cleavage by furin. (FIG. 7A) Diagrammatic structure of full length human erythroferrone and potential subunits generated after furin cleavage. (FIG. 7B) Coomassie blue staining of wild-type and an RARR-AAAA(A4) Erfe mutant, in the presence or absence of furin protease. The second in silico-predicted furin cleavage site does not appear to be active in the cell-types that were used in these experiments (arrows indicate ERFE).

FIG. 8. Erythroferrone subunits designed based on predicted patterns of furin cleavage differ in their ability to suppress hepcidin: (FIG. 8A) mono-Fc-huERFE A4 construct suppresses hepcidin expression, (FIG. 8B) Huh7 cells were treated for 24 h with human erythroferrone subunits that were designed to represent all the potential Erfe subunits that could be formed assuming activity at both in silico-predicted furin cleavage sites. (see FIG. 7A). Gene expression measured by qRT-PCR. Data represents mean+standard deviation (n=3). ***p<0.001; ****p<0.0001.

FIG. 9. The N-terminal domain of erythroferrone suppresses BMP signalling and Hamp in vivo. Eight weeks old C57/BL6 mice were injected i.p. with 100 μg of the F2 subunit of human erythroferrone or saline (6 mice per group). Three hours after injection, mice were culled and blood and tissues harvested for analysis of liver gene expression (FIG. 9A), serum hepcidin and serum iron (FIG. 9B). Gene expression measured by qRT-PCR. *p<0.05; **p<0.01; ****p<0.0001

FIG. 10A/B: BMPs 2/6, 5, 6 and 7 can compete with a neutralising anti-erfe antibody for binding to Erfe.

FIG. 10A: BMPs 2/6, 5, 6 and 7 compete with cryptate-labelled neutralising anti-ERFE antibody ab 15.1 for binding to biotinylated monoFC-muErfe with varying degrees of efficacy, FRET assay.

FIG. 10B: Anti-erfe antibody 15.1 inhibits Erfe function in a dose-dependent fashion: HepG2 cells containing the hepcidin-NanoLuc reporter fusion were treated with neutralising anti-ERFE antibody ab 15.1, serially diluted in tripling dilutions from a starting concentration of 500 nM, in the presence of 20 nM monoFC- and 625 pM BMP6.

FIG. 11: Neutralising anti-ERFE antibody prevents ERFE-based suppression of BMP signalling of BMPs 5/6/7 in vitro.

FIG. 12: Erfe suppresses SMAD phosphorylation by BMP2 in abdominal preadipocytes but not gluteal preadipocytes: Western blotting for phospho- and total SMAD1/5/8 and β-actin control in abdominal preadipocytes (FIG. 12A), Western blotting for phospho- and total SMAD1/5/8 and β-actin control in gluteal preadipocytes (FIG. 12B).

FIG. 13: Induction of Erfe production in mice leads to increase in non-esterified fatty acid levels but does not affect triacylglycerides: Measurement of non-esterified fatty acids (NEFA) concentrations, FIG. 13A, and triglycerides (TAG) concentrations, FIG. 13B, determined enzymatically using in wild-type and ERFE knock out male mice following consecutive EPO injections at time points 0 h, 24 h and 48 h.

### DEFINITIONS

As used herein a “disease of iron metabolism” can include hemochromatosis, such as HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis. Diseases of iron metabolism also include myelodysplasia syndrome, hepcidin deficiency, transfusional iron overload, thalassemia, thalassemia intermedia, alpha thalassemia, beta thalassemia, delta thalassemia, sideroblastic anemia, porphyria, porphyria cutanea tarda, African iron overload, hyperferritinemia, ceruloplasmin deficiency, atransferrinemia. Diseases of iron metabolism additionally include anemia, for example congenital dyserythropoietic anemia, anemia of chronic disease, anemia of inflammation, anemia of infection, hypochromic microcytic anemia, iron-deficiency anemia, iron-refractory iron deficiency anemia, anemia of chronic kidney disease. Diseases of iron metabolism further include erythropoietin resistance, iron deficiency of obesity, benign or malignant tumors that overproduce hepcidin or induce its overproduction, conditions with hepcidin excess, Friedreich ataxia, gracile syndrome, Hallervorden-Spatz disease, Wilson's disease, pulmonary hemosiderosis, hepatocellular carcinoma, cancer, hepatitis, cirrhosis of liver, pica, chronic renal failure, insulin resistance, diabetes, diabetes Type I or diabetes Type II, insulin resistance, glucose intolerance, atherosclerosis, neurodegenerative disorders, multiple sclerosis, Parkinson's disease, Huntington's disease, and Alzheimer's disease

As used herein a “disease or disorder comprising abnormally high hepcidin levels and/or abnormally low iron”, can be for example anemia or example iron-refractory iron-deficiency anemia (IRIDA), anemia of chronic kidney disease, anemias due to tumors that secrete hepcidin, anemia of inflammation, anemia associated with disease or infection which may be acute or chronic, also diabetes (Type I or Type II), insulin resistance, glucose intolerance.

As used herein “a disease comprising abnormally low hepcidin levels and/or abnormally high iron levels”, can be for example in treating thalassemia such as alpha-thalassemia, beta-thalassemia, delta-thalassemia or a thalassemia coexisting with other hemoglobinopathies, for example: hemoglobin E/thalassemia, hemoglobin S/thalassemia: hemoglobin C/thalassemia, hemoglobin D/thalassemia, congenital dyserythropoietic anemia, adult and juvenile hereditary hemochromatosis, and chronic liver diseases such as chronic hepatitis B, hepatitis B, hepatitis C, alcoholic liver disease, or iron overload disease for example, iron overload or iron toxicity, iron-loading anemia, alcoholic liver diseases, chronic hepatitis C and hereditary hemochromatosis.

As used herein, “BMP”, bone morphogenetic protein, includes all mammalian species of native sequence BMP or BMP polypeptide having BMP activity or recombinant BMP or BMP polypeptide having BMP activity, including human, rat, mouse and chicken and includes any of the family members BMP 2, 2/6 heterodimer, 3, 4, 5, 6, 7, 8a, 8b, 9, 10, 11, 12, 13, 14, or 15. The term “BMP” is used to include variants, isoforms and species homologs of human BMP or BMP polypeptide having BMP activity. Antibodies for use in the present invention may, in certain cases, cross-react with BMP or BMP polypeptide having BMP activity from species other than human. In certain embodiments, the antibodies may be completely specific for human BMP or BMP polypeptide having BMP activity and may not exhibit non-human cross-reactivity

“BMP activity” or “activity” or “biological activity”, in the context of BMP or BMP polypeptide having BMP activity generally refers to the ability to increase or enhance, for example in a dose dependent or concentration dependent manner or in comparison to conditions where the BMP is absent, in-vivo or in-vitro, for example in a cell, a biological sample or sample of body fluid, for example plasma or serum; the hepcidin activity, hepcidin expression, hepcidin levels or concentration, serum and/or plasma hepcidin levels/concentration, hepcidin mRNA production or levels or concentration, hepcidin mRNA production or levels/concentration, hepatic hepcidin mRNA production or levels/concentration and/or the reduction in plasma and/or serum concentration of iron. “Biological activity”, “BMP activity” or “activity” in the context of BMP or BMP polypeptide having BMP activity additionally refers to the ability to increase in-vivo or in-vitro, as hereinbefore described, activation of downstream pathway(s) mediated by BMP activity, such as the BMP/SMAD pathway, or the expression, concentration, level, activity, mRNA production of HAMP, ID1, ID2, ID3, SMAD6, SMAD7, ATOH8 or the phosphorylation of or ratio of phosphorylated to un-phosphorylated SMAD1, SMAD5 or SMAD8. Determination of activity can be made by assay of hepcidin, iron, HAMP, ID1, ID2, ID3, SMAD6, SMAD7, ATOH8, SMAD phosphorylation as described herein. “BMP activity” or “activity” or “biological activity”, in the context of BMP also refers to the ability of BMP to bind to a BMP receptor in-vivo or in-vitro, for example in a cell, a biological sample or sample of body fluid and/or activate downstream pathway(s) mediated by BMP activity as herein described.

As used herein, the terms “Erythroferrone” and “ERFE” refer to Erythroferrone and variants thereof that retain at least part of the biological activity of Erythroferrone. As used herein, Erythroferrone includes all mammalian species of native sequence Erythroferrone, including human, rabbit, cynomolgus monkey rat, mouse and chicken. The terms “Erythroferrone” and “ERFE” are used to include variants, isoforms and species homologs of human Erythroferrone. Antibodies for use in the present invention may, in certain cases, cross-react with Erythroferrone from species other than human. In certain embodiments, the antibodies may be completely specific for human Erythroferrone and may not exhibit non-human cross-reactivity. The complete amino acid sequence of an exemplary human Erythroferrone has Genbank accession number: AHL84165.1 (and is designated herein as SEQ ID NO:1).

“ERFE activity” or “activity” or “biological activity”, in the context of ERFE or ERFE polypeptide having erythroferrone activity generally refers to the ability to bind to BMP, preferably (i) BMP2, (ii) BMP2/6 heterodimer, (iii) BMP4, (iv) BMP5, (v) BMP6 or (vi) BMP7, and/or inhibit BMP activity or to decrease in-vitro, for example in a biological sample or sample of body fluid, for example plasma or serum, or in-vivo, the hepcidin activity, hepcidin expression, hepcidin levels/concentration, serum and/or plasma hepcidin levels/concentration, hepcidin mRNA production or levels/concentration, hepcidin mRNA production or levels/concentration, hepatic hepcidin mRNA production or levels/concentration and/or the increase in plasma and/or serum concentration of iron.

The term “polypeptide having BMP activity”, encompasses a BMP polypeptide having BMP activity or a polypeptide fragment of or a polypeptide derived from BMP having BMP activity, preferably wherein the BMP is as defined herein, BMP 2, 2/6 heterodimer, 3, 4, 5, 6, 7, 8a, 8b, 9, 10, 11, 12, 13, 14, or 15, preferably (i) BMP2, (ii) BMP2/6 heterodimer, (iii) BMP4, (iv) BMP5, (v) BMP6 or (vi) BMP7.

The term “polypeptide having erythroferrone activity” encompasses an ERFE polypeptide having erythroferrone activity or a polypeptide fragment of or a polypeptide derived from ERFE having erythroferrone activity

As used herein, an “agonist” in the context of BMP acts to increase or enhance BMP activity. An agonist of BMP can bind to or interact with BMP or BMP polypeptide having BMP activity and increase or enhance BMP activity, for example a small molecule, or anti-BMP antibody. An agonist of BMP can bind to or interact with BMP or BMP polypeptide having BMP activity to inhibit or prevent binding of an antagonist or compete with the antagonist for the binding of BMP, for example by inhibiting or preventing or competing for binding at the same binding site on BMP or BMP polypeptide having BMP activity. In the context of antibodies or antigen-binding portion thereof, the agonist can be an anti-BMP or anti-BMP polypeptide having BMP activity antibody, binding to or competing for the same binding region or epitope as an antagonist on BMP, preferably (i) BMP2, (ii) BMP2/6 heterodimer, (iii) BMP4, (iv) BMP5, (v) BMP6 or (vi) BMP7. Alternatively an agonist of BMP or BMP polypeptide having BMP activity can bind to or interact with an antagonist of BMP or BMP polypeptide having BMP activity, preferably (i) BMP2, (ii) BMP2/6 heterodimer, (iii) BMP4, (iv) BMP5, (v) BMP6 or (vi) BMP7, to inhibit or prevent binding to BMP or BMP polypeptide having BMP activity or compete with the BMP or BMP polypeptide having BMP activity for the binding of the antagonist, for example by inhibiting or preventing or competing for binding at the same binding site on the antagonist or in the context of antibodies, such as an anti-antagonist antibody, for the same binding region or epitope on the antagonist; for example wherein the antagonist can be ERFE or an ERFE polypeptide having erythroferrone activity, hence the agonist may be an anti-ERFE or anti-ERFE polypeptide having erythroferrone activity antibody. As used herein, an “agonist” in the context of BMP or BMP polypeptide having BMP activity can also act to enhance the binding between BMP or BMP polypeptide having BMP activity and its BMP receptor. Alternatively the agonist may bind to a BMP receptor binding inhibitor or antagonist preventing the inhibitor/antagonist from interacting with or binding to the BMP receptor. In this context the agonist may interact or bind to an inhibitor antagonist of BMP receptor binding to prevent antagonism or inhibition or alternatively the agonist may interact or bind to either the BMP or BMP polypeptide having BMP activity and/or the receptor to effect an enhancement of interaction, for example an antibody bispecific for the receptor and BMP or BMP polypeptide having BMP activity, preferably (i) BMP2, (ii) BMP2/6 heterodimer, (iii) BMP4, (iv) BMP5, (v) BMP6 or (vi) BMP7.

As used herein, an “antagonist” in the context of BMP or BMP polypeptide having BMP activity acts to decrease or inhibit BMP activity. An antagonist of BMP can bind to or interact with BMP, or a BMP polypeptide having BMP activity, preferably (i) BMP2, (ii) BMP2/6 heterodimer, (iii) BMP4, (iv) BMP5, (v) BMP6 or (vi) BMP7, and decrease or inhibit BMP activity; for example wherein the agonist can be an anti-BMP antibody or antigen binding fragment thereof, anti-BMP polypeptide having BMP activity, antibody or antigen binding fragment thereof, ERFE or an ERFE polypeptide having erythroferrone activity. An antagonist of BMP can bind to or interact with BMP or a BMP polypeptide having BMP activity, preferably (i) BMP2, (ii) BMP2/6 heterodimer, (iii) BMP4, (iv) BMP5, (v) BMP6 or (vi) BMP7, (a) to inhibit or prevent binding to a BMP receptor, preferably a receptor for preferably (i) BMP2, (ii) BMP2/6 heterodimer, (iii) BMP4, (iv) BMP5, (v) BMP6 or (vi) BMP7, alternatively (b) to inhibit or prevent binding of an agonist or compete with the agonist for the binding of BMP, for example by inhibiting or preventing or competing for binding at the same binding site on BMP or BMP polypeptide having BMP activity or in the context of antibodies or antigen-binding portion thereof, such as an anti-BMP antibody, for the same binding region or epitope on BMP or BMP polypeptide having BMP activity. Alternatively an antagonist of BMP can bind to or interact with an agonist of BMP to inhibit or prevent binding to BMP or BMP polypeptide having BMP activity or compete with the BMP or BMP polypeptide having BMP activity for the binding of the agonist, for example by inhibiting or preventing or competing for binding at the same binding site on the agonist or in the context of antibodies, such as an anti-agonist antibody, for the same binding region or epitope on the agonist. An antagonist of BMP can bind to or interact with BMP or a BMP polypeptide having BMP activity, preferably (i) BMP2, (ii) BMP2/6 heterodimer, (iii) BMP4, (iv) BMP5, (v) BMP6 or (vi) BMP7 to inhibit or prevent binding of a BMP receptor or compete with the BMP receptor for the binding of BMP. Alternatively an antagonist of BMP can bind to or interact with a BMP receptor, preferably for (i) BMP2, (ii) BMP2/6 heterodimer, (iii) BMP4, (iv) BMP5, (v) BMP6 or (vi) BMP7 to inhibit or prevent binding of a BMP or compete with the BMP for the binding of BMP receptor.

As used herein, an “agonist” or “antagonist” as used in the context of Erythroferrone or a polypeptide having Erythroferrone activity refers to the ability of a molecule, for example an antibody or antigen-binding portion thereof which is able to bind to Erythroferrone polypeptide having Erythroferrone activity, to enhance or inhibit Erythroferrone biological activity and/or downstream pathway(s) mediated by Erythroferrone activity. This encompasses molecules such as antibodies or antigen-binding portion thereof that can enhance, increase (including significantly), agonise or alternatively block, antagonize, suppress or reduce (including significantly) Erythroferrone biological activity, including downstream pathways mediated by Erythroferrone activity, such as hepcidin activity, hepcidin expression, hepcidin levels, serum and/or plasma hepcidin levels, hepcidin mRNA production or levels, hepcidin mRNA production or levels, hepatic hepcidin mRNA production or levels. As used herein, an “antagonist” as used in the context of an antibody or antigen-binding portion which binds to, specifically binds to or selectively binds to ERFE or an ERFE polypeptide having erythroferrone activity thereof or an “anti-ERFE antibody” or anti-“ERFE antagonist antibody” refers to an antibody or antigen-binding portion thereof which is able to bind to ERFE and inhibit ERFE biological activity and/or downstream pathway(s) mediated by ERFE activity and/or ability to bind to BMP or polypeptide having BMP activity, preferably (i) BMP2, (ii) BMP2/6 heterodimer, (iii) BMP4, (iv) BMP5, (v) BMP6 or (vi) BMP7 and/or inhibit BMP activity. Such antagonist antibodies encompass antibodies or antigen-binding portion thereof that can block, antagonize, suppress or reduce (including significantly) ERFE biological activity, including downstream pathways mediated by Erythroferrone ERFE, such as hepcidin activity, hepcidin expression, hepcidin levels, serum and/or plasma hepcidin levels, hepcidin mRNA production or levels, hepcidin mRNA production or levels, hepatic hepcidin mRNA production or levels. For the purposes of the present invention, it will be explicitly understood that the term “anti-ERFE antagonist antibody” or antigen-binding portion thereof encompass all the herein identified terms, titles, and functional states and characteristics whereby ERFE itself, and ERFE biological activity (including but not limited to its ability to mediate any aspect of hepcidin activity, expression or mRNA production and/or the increase in plasma and/or serum concentration of iron), or the consequences of the activity or biological activity, are substantially nullified, decreased, or neutralized in any meaningful degree. In some embodiments, an anti-ERFE antibody or anti-ERFE antagonist antibody or antigen-binding portion thereof binds ERFE and prevents ERFE BMP binding, preferably (i) BMP2, (ii) BMP2/6 heterodimer, (iii) BMP4, (iv) BMP5, (v) BMP6 or (vi) BMP7, prevents induced inhibition of BMP activity, prevents BMP induced hepcidin activity, expression or mRNA production and/or the increase in plasma and/or serum concentration of iron. Examples of anti-ERFE antibodies or anti-ERFE antagonist antibodies are provided herein, for example antibody ab 15.1.

According to the present invention the term “selectively binds” “selectively interacts”, “selectively recognises”, in the context of an antibody or binding portion thereof which binds or interacts with BMP or polypeptide having BMP activity, or with ERFE or an ERFE polypeptide having erythroferrone activity means that the antibody binds to a BMP family member or a specific sequence or epitope on said BMP family member or with greater affinity, avidity, and/or more readily, and/or with greater duration than it binds to other BMP family members or specific sequence or epitope on said other BMP family members. In the context of an antibody or binding portion thereof which binds or interacts with ERFE or an ERFE polypeptide having erythroferrone activity it means that the antibody binds to a specific sequence or epitope on said ERFE or an ERFE polypeptide with greater affinity, avidity, and/or more readily, and/or with greater duration than it binds to other ERFE or an ERFE polypeptides or specific sequence or epitope on said other ERFE or an ERFE polypeptides.

According to the present invention the term “specifically binds” “specifically interacts”, “specifically recognises”, an antibody or binding portion thereof which binds or interacts with ERFE or an ERFE polypeptide having erythroferrone activity it means that the antibody preferentially binds the ERFE or ERFE polypeptide or epitope thereof with greater affinity, avidity, more readily, and/or with greater duration than it binds to other isolated ERFE polypeptides of different sequence or epitope thereof, and/or does not significantly bind such other ERFE polypeptides of different sequence at high antibody concentrations for example in excess of the Kd for example at least or more than 2, 4, 6, 8, 10 fold in excess of Kd. In the context of an antibody which binds BMP this means that the antibody preferentially binds the BMP or polypeptide having BMP activity or BMP region or epitope thereof with greater affinity, avidity, more readily, and/or with greater duration than it binds to other BMP or polypeptide having BMP activity or BMP region or epitope thereof of different sequence or epitope thereof, and/or does not significantly bind such other BMP or polypeptide having BMP activity or BMP region or epitope thereof regions of different sequence at high antibody concentrations for example in excess of the Kd for example at least or more than 2, 4, 6, 8, 10 fold in excess of Kd. It is also understood by reading this definition that, for example, an antibody (or moiety or epitope) that specifically or preferentially binds, interacts with or recognises a first target may or may not specifically or preferentially bind, interact with or recognise a second target. As such, “specific binding” or “preferential binding” does not necessarily require (although it can include) exclusive binding. Generally, but not necessarily, reference to binding means preferential binding.

Binding selectivity in the context of antibody ligand interaction is a relative or comparative term indicating that the antibody can bind with differing affinities with different ligands to form a complex. For example, where an antibody is described as selectively binding BMP or polypeptide having BMP activity or ERFE or an polypeptide having erythroferrone activity this indicates that in comparison to binding an other BMP or polypeptide having BMP activity or ERFE or an polypeptide having erythroferrone activity the equilibrium constant for the reaction of displacement of BMP or ERFE polypeptides from the binding site of the antibody lies in the direction of the BMP or polypeptide having BMP activity or ERFE or an polypeptide having erythroferrone activity of the selective-antibody complex in comparison to the antibody complex with the other BMP or ERFE polypeptides.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also any antigen binding fragment (i.e., “antigen-binding portion”) or single chain thereof, fusion proteins comprising an antibody, and any other modified configuration of the immunoglobulin molecule that comprises an antigen recognition site including, for example without limitation, scFv, single domain antibodies (e.g., shark and camelid antibodies), maxibodies, minibodies, intrabodies, diabodies, triabodies, tetrabodies, v-NAR and bis-scFv (see, e.g., Hollinger and Hudson, 2005, Nature Biotechnology 23(9): 1126-1136). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody, as used herein, refers to one or more fragments of an intact antibody that retain the ability to specifically bind to an antigen or antigen epitope, for example to BMP or a polypeptide having BMP activity or ERFE or polypeptide having erythroferrone activity. Antigen binding functions of an antibody can be performed by fragments of an intact antibody. Examples of binding fragments encompassed within the term “antigen binding portion” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., 1989 Nature 341:544-546), and an isolated complementarity determining region (CDR).

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987). When choosing FR to flank subject CDRs, e.g., when humanizing or optimizing an antibody, FRs from antibodies which contain CDR1 and CDR2 sequences in the same canonical class are preferred.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., 1989, Nature 342:877-883. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., 1996, J. Mol. Biol., 262:732-745. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

The term “monoclonal antibody” (Mab) refers to an antibody, or antigen-binding portion thereof, that is derived from a single copy or clone, including e.g., any eukaryotic, prokaryotic, or phage clone, and not the method by which it is produced. Preferably, a monoclonal antibody of the invention exists in a homogeneous or substantially homogeneous population.

“Humanized” antibody refers to forms of non-human (e.g. murine or chicken) antibodies, or antigen-binding portion thereof, that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity.

“Human antibody or fully human antibody” refers to those antibodies, or antigen-binding portion thereof, derived from transgenic mice carrying human antibody genes or from human cells. The term “chimeric antibody” is intended to refer to antibodies, or antigen-binding portion thereof, in which the variable region sequences are derived from one species and the constant region sequences are derived from another species, such as an antibody in which the variable region sequences are derived from a mouse antibody and the constant region sequences are derived from a human antibody. Antibodies can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50 (1999) and Fellouse, F. A., et al, J. Mol. Biol., 373(4):924-40 (2007)).

In some embodiments, antibodies of the invention, or antigen-binding portion thereof, can comprise a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level and/or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jeffe s et al., Immunological Reviews 163:59-76, 1998. In some embodiments, the constant region is modified as described in Eur. J. Immunol., 1999, 29:2613-2624; PCT Application No. PCT/GB99/01441; and/or UK Patent Application No. 9809951.8.

In some embodiments, an antibody constant region can be modified to avoid interaction with Fc gamma receptor and the complement and immune systems. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692.

In some embodiments, the constant region can be modified as described in Eur. J. Immunol., 1999, 29:2613-2624; PCT Application No. PCT/GB99/01441; and/or UK Patent Application No. 9809951.8. In such embodiments, the Fc can be human IgG₂ or human IgG₄. The Fc can be human IgG2 containing the mutation A330P331 to S330S331 (designated IgG2Δa), in which the amino acid residues are numbered with reference to the wild type IgG2 sequence. Eur. J. Immunol., 1999, 29:2613-2624. In some embodiments, the antibody comprises a constant region of IgG comprising the following mutations (Armour et al., 2003, Molecular Immunology 40 585-593): E233F234L235 to P233V234A235 (IgG₄Δc), in which the numbering is with reference to wild type IgG4. In yet another embodiment, the Fc is human IgG₄ E233F234L235 to P233V234A235 with deletion G236 (IgG₄Δb)—In another embodiment the Fc is any human IgG₄ Fc (IgG₄, IgGΔΔb or IgG Δ_{c}) containing hinge stabilizing mutation S228 to P228 (Aalberse et al., 2002, Immunology 105, 9-19).

In some embodiments, the antibody comprises a human heavy chain IgG2 constant region comprising the following mutations: A330P331 to 53305331 (amino acid numbering with reference to the wild type IgG2 sequence). Eur. J. Immunol., 1999, 29:2613-2624. In still other embodiments, the constant region is aglycosylated for N-linked glycosylation. In some embodiments, the constant region is aglycosylated for N-linked glycosylation by mutating the oligosaccharide attachment residue and/or flanking residues that are part of the N-glycosylation recognition sequence in the constant region. For example, N-glycosylation site N297 may be mutated to, e.g., A, Q, K, or H. See, Tao et al., J. Immunology 143: 2595-2601, 1989; and Jefferis et al., Immunological Reviews 163:59-76, 1998. In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications comprised by the antibodies of the invention, or antigen-binding portion thereof, include antibodies that have been modified as described in PCT Publication No. WO 99/58572. These antibodies comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcγRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

In some embodiments, the antibodies of the invention, or antigen-binding portion thereof, comprises a modified constant region that has increased binding affinity for FcRn and/or an increased serum half-life as compared with the unmodified antibody.

In a process known as “germlining”, certain amino acids in the VH and VL sequences can be mutated to match those found naturally in germline VH and VL sequences. In particular, the amino acid sequences of the framework regions in the VH and VL sequences can be mutated to match the germline sequences to reduce the risk of immunogenicity when the antibody is administered. Germline DNA sequences for human VH and VL genes are known in the art (see e.g., the “Vbase” human germline sequence database; see also Kabat, E. A., et al., 1991, Sequences of Proteins of Immunological Interest, Fifth Edition, U.S. Department of Health and Human Services, NIH Publication No. 91-3242; Tomlinson et al., 1992, J. Mol. Biol. 227:776-798; and Cox et al., 1994, Eur. J. Immunol. 24:827-836).

Another type of amino acid substitution that may be made is to remove potential proteolytic sites in the antibody. Such sites may occur in a CDR or framework region of a variable domain or in the constant region of an antibody. Substitution of cysteine residues and removal of proteolytic sites may decrease the risk of heterogeneity in the antibody product and thus increase its homogeneity. Another type of amino acid substitution is to eliminate asparagine-glycine pairs, which form potential deamidation sites, by altering one or both of the residues. In another example, the C-terminal lysine of the heavy chain of an antibody of the invention can be cleaved. In various embodiments of the invention, the heavy and light chains of the antibodies may optionally include a signal sequence.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

The term “epitope” refers to that portion of a molecule capable of being recognized by and bound by an antibody, or antigen-binding portion thereof, at one or more of the antibody's antigen-binding regions. Epitopes can consist of defined regions of primary secondary or tertiary protein structure and includes combinations of secondary structural units or structural domains of the target recognised by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody can specifically bind as determined by any method well known in the art, for example, by conventional immunoassays, antibody competitive binding assays or by x-ray crystallography or related structural determination methods (for example NMR). A “nonlinear epitope” or “conformational epitope” comprises noncontiguous polypeptides (or amino acids) within the antigenic protein to which an antibody specific to the epitope binds. Once a desired epitope on an antigen is determined, it is possible to generate antibodies to that epitope, e.g., using the techniques described in the present specification. During the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another e.g., the antibodies compete for binding to the antigen or antigenic epitope.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “binding affinity” or “K_{D}” as used herein, is intended to refer to the dissociation rate of a particular antigen-antibody interaction. The K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M). It follows that the smaller the K_{D}, the stronger the affinity of binding. Therefore, a K_{D} of 1 μM indicates weak binding affinity compared to a K_{D} of 1 nM. K_{D} values for antibodies can be determined using methods well established in the art. One method for determining the K_{D} of an antibody is by using surface plasmon resonance (SPR), typically using a biosensor system such as a Biacore® system.

The term “potency” is a measurement of biological activity and may be designated as IC₅₀, or effective concentration of an antibody to an antigen, for example BMP or polypeptide having BMP activity or ERFE or polypeptide having erythroferrone activity, which is required to inhibit 50% of activity measured in a ERFE or BMP activity assay such as described herein.

The term “inhibit” or “neutralize” as used herein with respect to biological activity of an antibody of the invention means the ability of the antibody to substantially antagonize, prohibit, prevent, restrain, slow, disrupt, eliminate, stop, reduce or reverse e.g. progression or severity of that which is being inhibited including, but not limited to, a biological activity or expression of BMP or polypeptide having BMP activity or ERFE or polypeptide having erythroferrone activity.

The term “compete”, as used herein with regard to an antibody or antigen-binding portion thereof, means that a first antibody, or an antigen-binding portion thereof, binds to an epitope, in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s) or binding region(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein. The term “compete(s)” as used herein encompasses antibodies or antigen binding portions thereof binding BMP or polypeptide having BMP activity or ERFE or polypeptide having erythroferrone activity, or epitopes thereof.

According to the invention BMP or erythroferrone activity and agonism or antagonism thereof can be determined by assay of hepcidin activity, hepcidin expression, hepcidin levels, serum and/or plasma hepcidin levels, hepcidin mRNA production or levels, hepatic hepcidin mRNA production or levels optionally as compared to a control, for example in absence of the BMP or erythroferrone or agonist or antagonist thereof. Assay can be made in-vivo and/or in-vitro, for example in a biological sample or sample of body fluid, for example plasma or serum, urine, saliva, cerebral spinal fluid, spinal fluid, blood, cord blood, amniotic fluid or peritoneal dialysis fluid. Alternatively an assay may be made in an in-vitro system such as the nano-luc assay described herein. Hepcidin expression, levels and/or concentration may be determined using an enzyme-linked immunosorbent assay (ELISA) or a competitive enzyme-linked immunosorbent assay (ELISA) performed in the presence of labelled hepcidin. The assay can comprise a surface bound hepcidin ligand such as an antibody or ligand binding domain thereof to capture hepcidin from the sample, and a further such ligand optionally conjugated to an enzyme readout in a substrate reaction or conjugated to an alternative means of signal generation which may be chromogenic or chemifluorescent or chemiluminescent. A competitive ELISA can be used and can comprise an unlabeled hepcidin primary ligand, for example an anti-hepcidin antibody, which is incubated with a sample containing the hepcidin antigen for measurement. The primary ligand-antigen complexes are then added to a container pre-coated with hepcidin antigen. Unbound primary ligand is removed by washing. The more hepcidin antigen in the sample, the less ligand will be able to bind to the antigen in the container. A secondary ligand for example an antibody that is specific to the primary ligand/antibody and conjugated with an enzyme or equivalent readout means is added and optionally subsequently a substrate is added to elicit a chromogenic or fluorescent signal. Determination of iron levels may be made in-vitro, for example in a biological sample or sample of body fluid, as herein before described, for example by ferritin assay. Ferritin can be assayed for example by Radioimmunoassay (RIA) or Immunoradiometric assay, IRMA. In a radioimmunoassay, a known quantity of ferritin is labeled with a radioactive isotope then mixed with a known amount of anti-ferritin antibody the sample containing an unknown quantity of ferritin is subsequently added to compete for binding and the ratio of antibody-bound radiolabeled ferritin to free radiolabeled ferritin is determined which when performed at varying concentrations of labeled ferritin permits the calculation of unlabeled ferritin in the sample. In IRMA, the antibodies are labeled with radioisotopes which are used to bind ferritin present in the sample, remaining labeled antibodies are removed by a second reaction with a solid phase ferritin. The amount of radioactive remaining in the solution is direct function of the ferritin concentration.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

As used herein, “pharmaceutically acceptable carrier” or “pharmaceutical acceptable excipient” includes any material which, when combined with an active ingredient, allows the ingredient to retain biological activity and is non-reactive with the subject's immune system. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “treating”, as used herein, unless otherwise indicated, means reversing, alleviating, inhibiting the progress of, delaying the progression of, delaying the onset of, or preventing or inhibiting the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the avoidance of doubt, reference herein to “treatment” includes reference to curative, palliative and prophylactic treatment. For the avoidance of doubt, references herein to “treatment” also include references to curative, palliative and prophylactic treatment.

A “biological sample” encompasses a variety of sample types obtained from an individual and can be used in a diagnostic or monitoring assay. The definition encompasses blood, plasma, serum, urine and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples.

As used herein, “substantially pure” refers to material which is at least 50% pure (i.e., free from contaminants), more preferably at least 90% pure, more preferably at least 95% pure, more preferably at least 98% pure, more preferably at least 99% pure.

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present invention. The materials, methods, and examples are illustrative only and not intended to be limiting.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims. The contents of all figures and all references, patents and published patent applications cited throughout this application are expressly incorporated herein by reference.

### EXAMPLES

### Example 1. BMP Effect on Hepcidin Expression

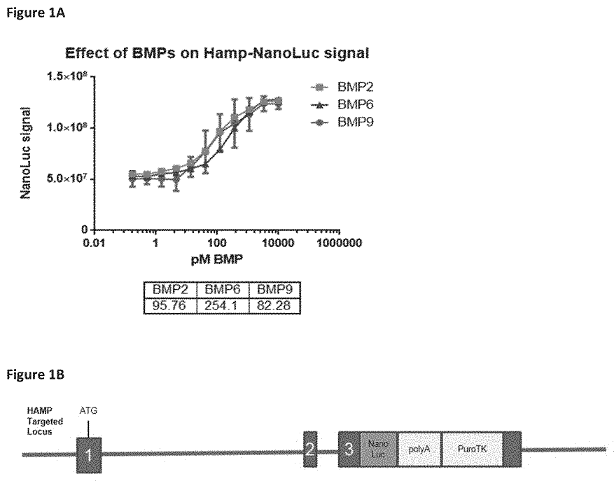

The was measured in-vitro using as follows: A hepcidin transcriptional fusion reporter assay (nano-luc or nano-luciferase assay) was developed to assess the effect of BMPs on hepcidin production in-vitro. A nanoluciferase reporter was inserted at the end of the coding sequence of the endogenous HAMP gene, in HepG2 hepatoma cells, creating a HAMP-nanoluciferase fusion. CRISPR-Cas9 was used to edit the cells with this knock-in reporter construct. A bovine Growth Hormone poly A was included after the nanoLuc to ensure proper mRNA processing. A Puromycin-TK expression cassette was also included 3′ of the nanoluc reporter to enrich for properly targeted clones. Clones that were homozygous for the HAMP-NanoLuc fusion were selected and tested for their response to BMPs and LDN(4-[6-[4-(1-Piperazinyl)phenyl]pyrazolo[1,5-a]pyrimidin-3-yl]quinoline dihydrochloride), a potent ALK2/3 BMP inhibitor, a schematic of the nano-luc construct is shown in FIG. 1B. The effect of representative BMPs (BMP, 2, 6, 9) on the Hamp-nanoLuc signal was assessed. Approximately 20,000 cells (cells split with trypsin/EDTA) were suspended in 200 uL of complete media (DMEM+10% serum+Na pyruvate) and were distributed into each well of 96-well plates and incubated for 24 hours at 37 C. Media was then aspirated and the cells treated in 100 uL complete media for 24 h at 37 C in the presence of the selected BMP which was serial diluted (in duplicates), controls of fresh media alone were used for a baseline signal (in duplicate). Following the incubation 90 uL of supernatant was transferred to a white wall 96 well plate and equilibrated at RT, 90 uL of RT NanoLuc reagent was added/well (for 1 plate, 10 mL diluting reagent+200 uL NanoLuc substrate—from Nano-Glo Luciferase Assay System, Promega, ref cat #N1150) and mixed 2-3 min at room temperature. Plates were read on Envision with Lum700 program following the manufacturer's instructions. Data shown in FIG. 1A demonstrates that there is a dose-dependent effect of each assayed BMP on hepcidin expression in the assay system. In conclusion hepcidin expression is modulated via BMP.

### Example 2. BMP Interaction with ERFE Measured by Biacore

Iron absorption is tightly regulated by erythropoietic demand via control of hepcidin expression. Erythropoietin (EPO) causes hepcidin suppression, at least in part by increasing synthesis of the hormone erythroferrone (ERFE). ERFE is produced by erythroblasts after bleeding or EPO treatment, and acts on hepatocytes to suppress hepcidin expression and increase iron availability. ERFE knock out mice fail to suppress hepcidin after phlebotomy and show delayed recovery from blood loss. Furthermore, serum ERFE concentrations are increased in humans after blood loss and EPO administration, and in β-thalassemia patients. Hepcidin expression is modulated via the BMP/SMAD signalling pathway. In particular BMP6 and BMP2, produced by liver sinusoidal endothelial cells can trigger a signalling cascade by binding to BMP receptors on hepatocyte cell membranes, which phosphorylate cytosolic SMADs (SMAD1/5/8) that translocate to the nucleus complexed with SMAD4 to activate the transcription of target genes, including hepcidin (HAMP). We sought to determine whether the mechanism by which ERFE suppresses hepcidin is through interaction with BMP. Binding constants were measure using Biacore SPR. BMPs 2, 4, 6 at 50 nM was run against ERFE A4-biotin amine coupled to CM4 sensor chip in MES-Tris buffer pH6.0, 25° C.

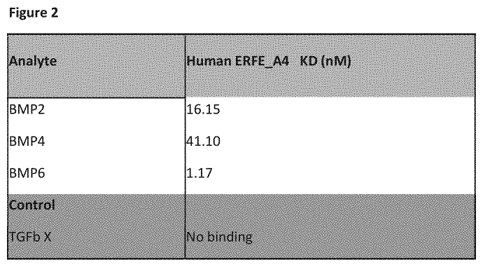

In conclusion, the data in FIG. 2 shows that ERFE binds with nanomolar Kd to the selected BMPs 2, 4 and 6, most tightly to BMP 6 as determined by SPR (Biacore™).

### Example 3. BMP/SMAD Signalling Pathway is Suppressed by Erythroferrone In-Vitro

(a) Hepcidin expression is modulated via the BMP/SMAD signalling pathway: BMPs, for example BMP6 and BMP2, produced by liver sinusoidal endothelial cells, trigger a signalling cascade by binding to BMP receptors on hepatocyte cell membranes, which phosphorylate cytosolic SMADs (SMAD1/5/8) that translocate to the nucleus complexed with SMAD4 to activate the transcription of target genes, including hepcidin (HAMP).

(b) Gene expression microarray: Microarray analysis of Huh7 cells treated with mouse or human erythroferrone was carried out to test the activity of erythroferrone on the BMP/SMAD signalling pathway. Generally cell treatments were carried out according to the following protocol: Huh7 and HepG2 were cultured in Dulbecco's Modified Eagle's Medium—High Glucose (Sigma), supplemented with 10% Fetal Bovine Serum (Sigma), 1% Penicillin-Streptomycin (Sigma) and 1% L-Glutamine (Sigma), unless otherwise indicated. Cells were plated 24 h before treatments in 24-well (gene expression analysis) or 12-well (protein analysis) cell culture plates. At the time of treatment, cells were washed with PBS and fresh media was added. Cells were treated with recombinant human or mouse ERFE (10 μg/ml), BMP2, 4, 5, 6, 7 or 9 (R&D systems), Activin B (R&D systems), LDN-193189 (MedChem Express) or IL-6 (R&D systems), for 30 minutes (Western Blot), 6 or 24 hours (gene expression).

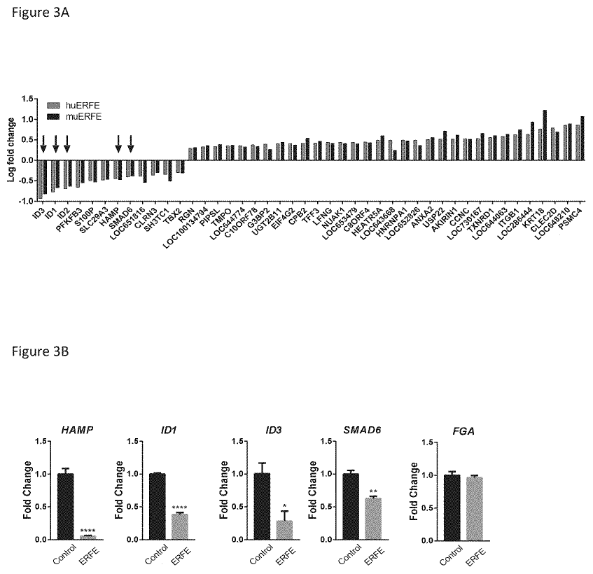

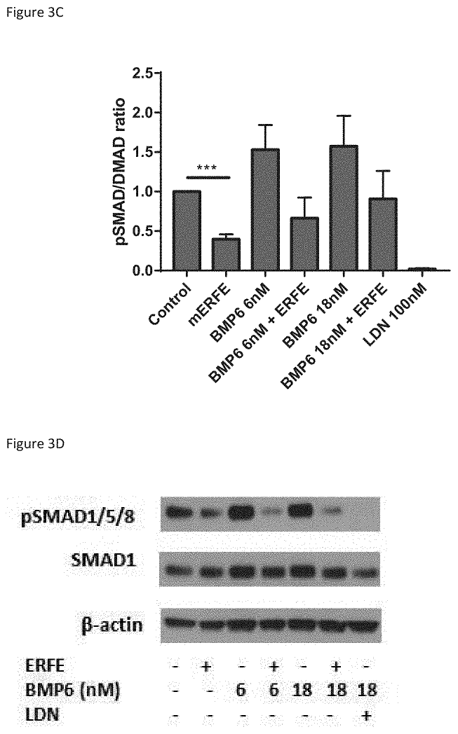

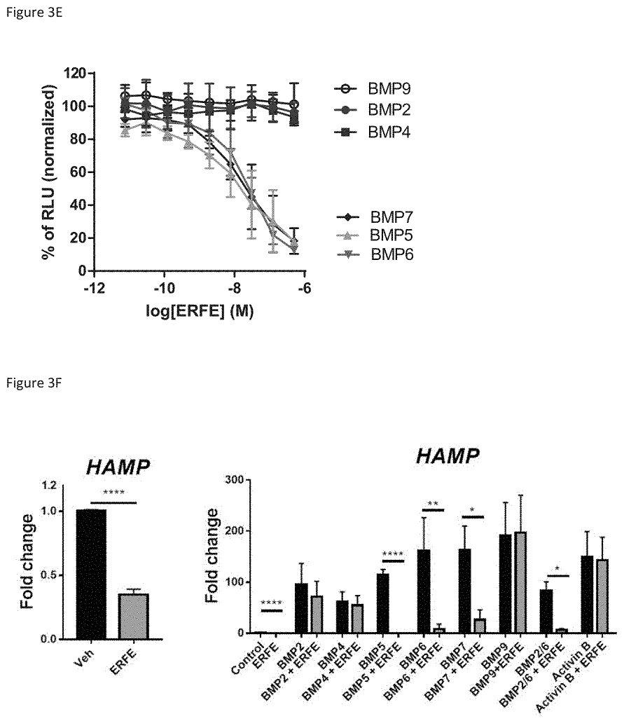

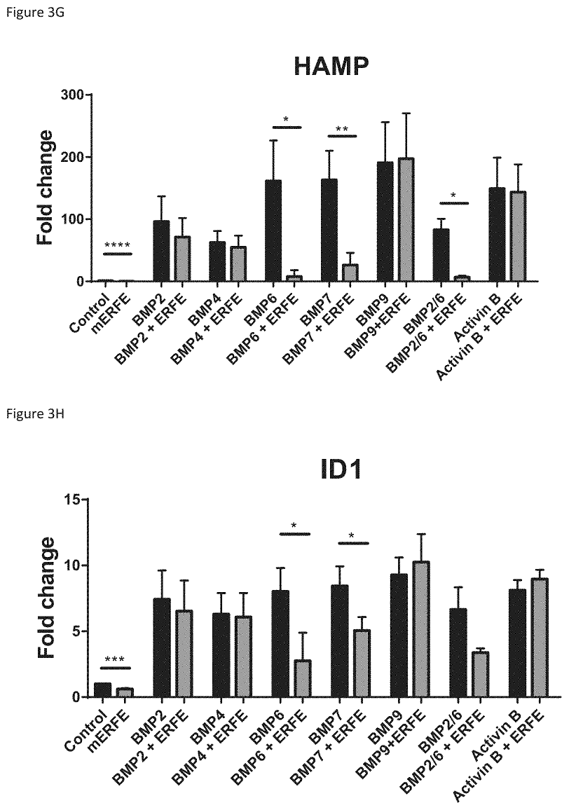

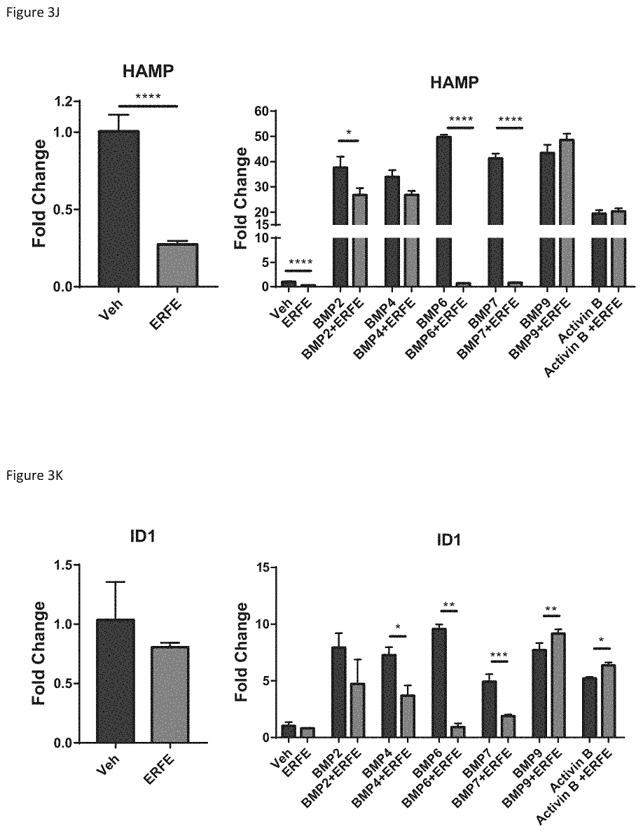

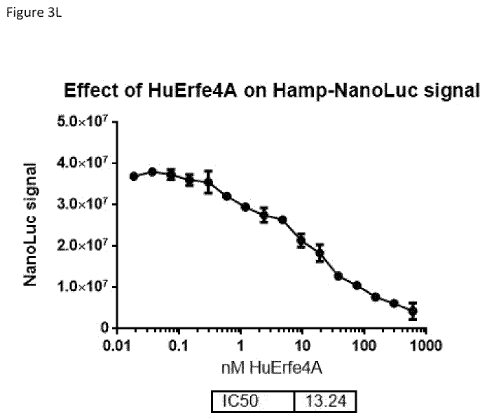

For the purposes of gene expression microarray analysis RNA was isolated from the Huh7 cells using RNeasy Plus kit (Qiagen), this was followed by RNA quantification and quality assessment using a 2100 Bioanalyzer (Agilent). RNA was converted into biotin labelled cRNA for hybridization and gene expression analysed using the Human HT12v4.0 Expression Beadchip (Illumina) and the Illuminas's iScan Scanner. Raw data was normalised using the lumi package (Bioconductor) and compared using LIMMA (Bioconductor). Statistical significance was set at p<0.05. Analysis of the cells treated with mouse or human ERFE revealed 12 suppressed genes (FIG. 3A), 5 of which are targets of BMP/SMAD signalling—ID1, ID2, ID3, SMAD6 and HAMP. No changes were observed in target genes of the JAK/STAT3 pathway, a canonical pathway that influences hepcidin expression, indicating that reduction in inflammatory signalling is not the primary driver of Erfe mediated reduction of HAMP expression.

(c) RNA isolation, cDNA synthesis and qRT-PCR: Suppression of several BMP-target genes: HAMP, ID1, ID3, SMAD6 and SMAD7 was further confirmed by qRT-PCR. Cells were lysed and RNA was isolated using the RNeasy Plus kit (Qiagen), followed by RNA quantification and quality assessment using Nanodrop (Thermo Fisher). cDNA was synthesized using the High Capacity RNA-to-cDNA kit (Applied Biosystems). Gene expression was assessed using quantitative real-time PCR with Taqman Gene Expression Master Mix and inventoried Taqman Gene expression assays (Applied Biosystems) specific for the genes of interest according to the manufacturer's instructions. Data shown in FIG. 3B demonstrates gene expression measured by qRT-PCR, performed using the QuantStudio 7 Flex Real-Time PCR system, relating to the five selected BMP/SMAD target genes and FGA (a JAK/STAT3 target gene) in Huh7 cells treated with vehicle or mouse ERFE (10 μg/ml). In conclusion the changes in HAMP are not mediated by decreased inflammatory signalling, no changes were observed in FGA, a target gene of the JAK/STAT3 pathway.

(d) ERFE effects on BMP/SMAD signalling measured by SMAD 1/5/8 phosphorylation: To confirm the suppression of BMP/SMAD signalling, we analysed phosphorylation of SMAD1/5/8, required to transduce the signal leading to HAMP upregulation. Analysis was carried out by Western blot according to the following methodology: Cells were lysed at 4° C. using RIPA buffer (Thermo Scientific) containing protease/phosphatase inhibitor (Cell Signalling). Lysates were denatured at 95° C. and separated on a 10% SDS polyacrylamide gel (Bio-Rad), following the manufacturer's instruction. Protein sizes were estimated by using the Novex Sharp Pre-Stained Protein Ladder (life technologies). Protein was transferred to a nitrocellulose membrane, then blocked with milk/TBS for 1 h. Antibodies used were anti-P-SMAD 1/5 (S463s/465)/9(S465/467) (Cell signalling 13820S 1:500), anti-Smad1 (Cell Signalling 6944S 1:500), anti-β-actin-peroxidase (Sigma A3854 1:10 000), and Anti-rabbit IgG HRP conjugated (RnD systems HAP008 1:5000). ERFE was shown to cause a decrease in SMAD 1/5/8 phosphorylation relative to non-treated cells (FIG. 3C), both at baseline and after BMP6 stimulation. In this case Huh7 cells were treated with mouse ERFE (10 μg/ml) or BMP6 and LDN, a small molecule inhibitor of BMPs, (100 nM), alone or in combination, for 30 min and pSMAD/SMAD ratios values were calculated by densitometry. Erythroferrone caused a decrease in SMAD phosphorylation relative to non-treated cells (FIG. 3D). Furthermore, erythroferrone also blunted the increase in phosphorylation caused by BMP6 stimulation.

Altogether, these data suggest that erythroferrone suppresses hepatic HAMP via suppression of BMP/SMAD signalling.

As HAMP can be stimulated by a variety of ligands, we further tested the effect of ERFE in cells treated with various BMPs by measuring BMP/SMAD signalling output in C2C12 Bre-Luc cells. C2C12 Bre-Luc cells were treated with 2 nM of BMP in combination with a gradient of mouse ERFE concentrations (7.5 pM to 0.5 μM) for 24 h, and luminescence measured in each well. Data was normalized to percentage of maximum luminescence (no ERFE) and is shown in FIG. 3E. We observed a dose-dependent decrease in activation of BMP signalling by BMP5, BMP6 and BMP7 in response to increased concentrations of ERFE with no effect in BMP2, BMP4 and BMP9 activity, and this effect was seen in the different cell types tested as discussed below. Analogous data confirmed suppression of HAMP and ID1 in cells treated with ERFE and BMP 2/6, BMP5, BMP6 and BMP7, FIG. 3F.

In conclusion, these data demonstrate that ERFE inhibits BMP/SMAD signalling, specifically affecting activation by BMP5, BMP6 and BMP7, (and to some degree BMP 2/6) leading to hepcidin suppression.

(e) ERFE suppresses BMP/SMAD signalling in-vitro in a variety of cell types primarily by inhibiting BMP5, BMP6 and BMP7.

The inhibitory effect of EFE on selected BMPs was tested in-vitro in three different cell types, C2C12, Huh7 and HepG2. Huh7 (FIG. 3G, 3H) and HepG2 (FIGS. 3J, 3K) cells were treated with 2 nM of BMPs, alone or in combination with 10 μg/ml of mouse ERFE, in serum-free media, and analysed 6 h after treatment. Gene expression of HAMP and ID1 was measured by qRT-PCR. Results expressed as fold change relative to non-treated cells from 3 independent experiments. Statistical significance was analysed for each pair of BMP treatments. (*p<0.05, **p<0.01, ***p<0.001, ****p<0.0001, Student's t test). Additionally we tested the effect of ERFE in cells treated with various BMPs by measuring BMP/SMAD signalling output in C2C12 Bre-Luc cells (FIG. 3E). C2C12 Bre-Luc cells were treated with 2 nM of BMP in combination with a gradient of mouse ERFE concentrations (7.5 pM to 0.5 μM) for 24 h, and luminescence measured in each well.

Human ERFE was further shown to have a dose dependent effect on hepcidin production in the NanoLuc system. NanoLuc cells were prepared as described in Example 1 and seeded into a multiwall dish at a density of 20000 cells/well. monoFC-huErfe was serially diluted in two fold dilutions from a starting concentration of 600 nM, data is shown in FIG. 3L, from this it is concluded that ERFE suppression of hepcidin is dose dependent.

In conclusion, taken together these data demonstrate that ERFE suppresses BMP/SMAD signalling by inhibiting BMP5, BMP6 and BMP7 and that suppression of hepcidin is dose dependent. Inhibitory action is also seen for BMP2 and 4 and BMP2/6 heterodimer in a variety of different cell types.

### Example 4. BMP/SMAD Signalling Pathway is Suppressed by Erythroferrone In-Vivo

(a) Animal studies: Wild-type male C57BL/6 mice were purchased from Harlan Laboratories, UK. Embryos from Fam132b+/− mice on a mixed Sv129/C57BL/6 background were obtained from the Mutant Mouse Regional Resource Center (MMRRC) at UC Davis (strain 66; 12955-Fam132btm1Lex/Mmucd, ID MMRRC:032289-UCD) and backcrossed onto C57BL/6 background using marker-assisted accelerated backcrossing. Heterozygote pairs were mated to generate homozygous animals from which knockout and wild-type colonies were maintained. Animals were housed in individually ventilated cages in the Department of Biomedical Services, University of Oxford, and provided access to normal chow (163 ppm of iron, Special Diets Services 801700) and water ad libitum. All experiments were performed in 9-13 weeks old male mice. For EPO treatments, mice were injected intraperitoneally with 200 IU recombinant human EPO (Bio-Rad) in water or vehicle (water) daily for three consecutive days and culled 24 h after the last EPO injection. For ERFE treatments, mice were injected intravenously with 200 μg of recombinant mouse ERFE or vehicle (saline) and culled 3 h after treatment. Mice were euthanized in increasing CO₂ concentrations.

(b) Serum iron analysis: Blood was taken by cardiac puncture immediately after euthanising mice and collected in BD EDTA or SST (serum) Microtainer tubes. Serum was prepared by centrifugation of clotted blood at 8000×g for 3 minutes in BD Microtainer SST tubes (Beckton Dickinson) and used for serum iron quantification using a Abbott Architect c16000 automated analyzer (Abbott Laboratories).

(c) Tissue non-heme iron measurement: Liver tissues were dried for 4 hours at 100° C., weighed and digested in 10% tricholoroacetic acid (Sigma)/30% hydrochloric acid (Sigma) for 20 hours at 65° C. A standard curve was generated using a dilution series of ferric ammonium citrate (Sigma) in the 10% (w/v) trichloroacetic acid/30% hydrochloric acid mixture. Non-heme iron content was determined colorimetrically by measuring absorbance at 535 nm following reaction with chromogen reagent containing 0.1% (w/v) bathophenoldisulphonic acid (Sigma)/0.8% thioglycolic acid (Sigma).

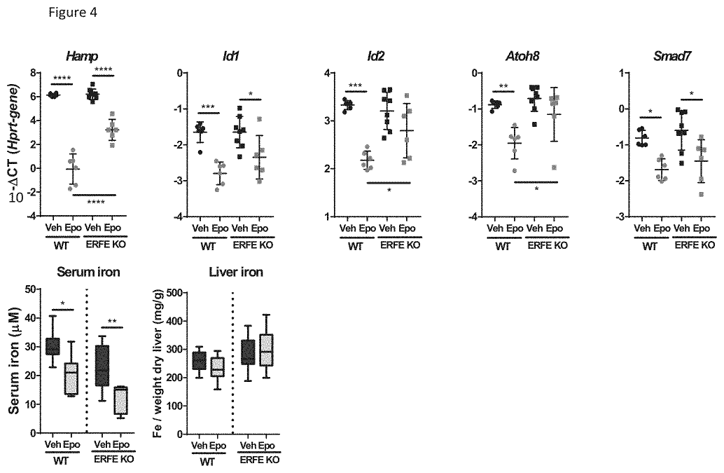

(d) Mice challenged with erythropoietin or ERFE downregulate BMP-target genes: WT and ERFE KO male mice (10-13 weeks old) were injected with 3 doses of 200u of EPO, one dose every 24 h, and analysed 24 h after the last injection. Expression of BMP-target genes in the liver was measured by qRT-PCR. EPO strongly suppressed Hamp and other BMP-target genes—Id1, Id2, Atoh8 and Smad7—indicating a decrease in BMP-signalling activity (FIG. 4). In ERFE KO mice, suppression of Hamp and BMP target genes was blunted or prevented, confirming that the effect of EPO on BMP signalling requires ERFE.

Observed partial contributions of ERFE-independent decrease in BMP-signalling could be explained by decreased serum iron in EPO-treated mice (FIG. 4) due to increased iron consumption by erythroblasts, analysis was 24 hours after last EPO injection, serum iron measured in a chemical analyser and liver iron measured using a colorimetric assay.

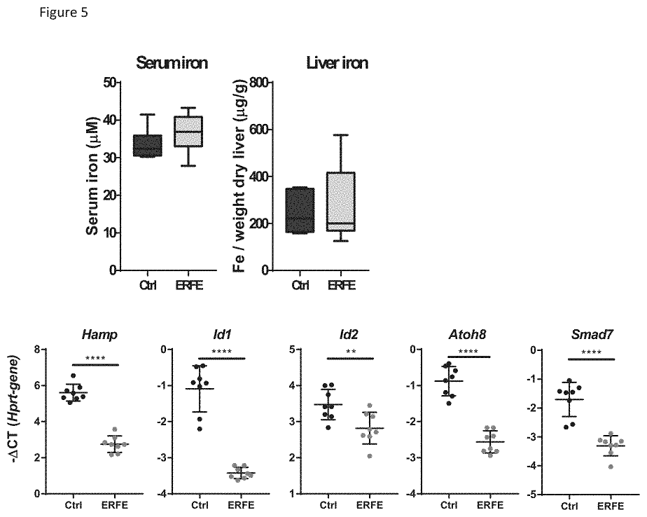

To distinguish between the effects of ERFE and iron on hepatic BMP signalling we performed a short-term ERFE treatment in WT mice (analysis was 3 hours after last EPO injection), nine weeks old WT male mice were injected i.v. with 200 μg of the murine ERFE (muERFE WT) or an inactive control (Clip huERFE) which is a mono Fc fusion with a 14 amino acid N-terminal region of human ERFE. Three hours after the injections, mice were culled and serum and liver iron measured. Serum and liver iron were not affected by ERFE injection (FIG. 5) indicating that iron levels are not driving the observed effect of decreased BMP signalling. Additionally, three hours after the injections, mice were culled and the liver harvested for analysis of liver gene expression for BMP-target genes. At this 3 hour time-point ERFE significantly reduced the expression of Hamp and BMP-target genes (FIG. 5). It was noted that serum hepcidin levels were also reduced in ERFE administered animals in comparison to the control.

In conclusion, ERFE suppresses BMP/SMAD signalling independently of iron, particularly by inhibiting BMP5, BMP6 and BMP7.

### Example 5. Erythroferrone Activity is not Mediated by the ERFE Globular C1q Domain

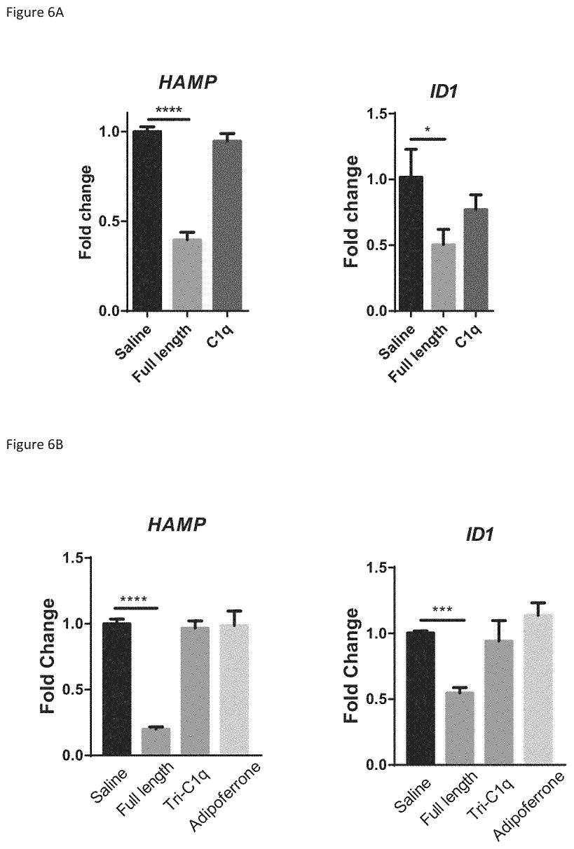

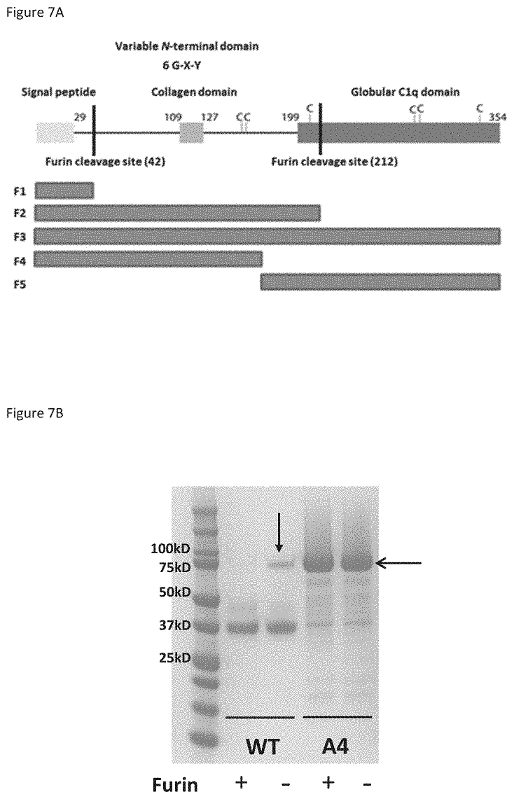

Erythroferrone is a member of the C1q/TNF-related protein (CTRP) family and comprises a structure composed of a signal peptide, an N-terminal, a collagen-like domain, and a globular C terminal domain homologous to complement protein 1q (C1q). To analyse the mode of action of erythroferrone, we investigated the activity of different subunits of the protein. We have generated a protein composed solely by the C1q domain and compared its activity with the full-length protein. We observed that the globular domain per se is not sufficient to suppress HAMP and ID1 in Huh7 cells (FIG. 6A). To test if the lack of effect is due to structural constraints due to the lack of the N-terminal domain, we generated C1q trimers (the most common arrangement in other members of the CTRP family), and a hybrid of the N-terminus of adiponectin (a structurally related member of the CTRP family) and the C1q domain of erythroferrone-adipoferrone (FIG. 6B).

In conclusion, none of the proteins tested above could suppress HAMP, confirming that the globular domain is not required for erythroferrone activity.

### Example 6. ERFE Activity is Mediated by an Active N-Terminal Domain

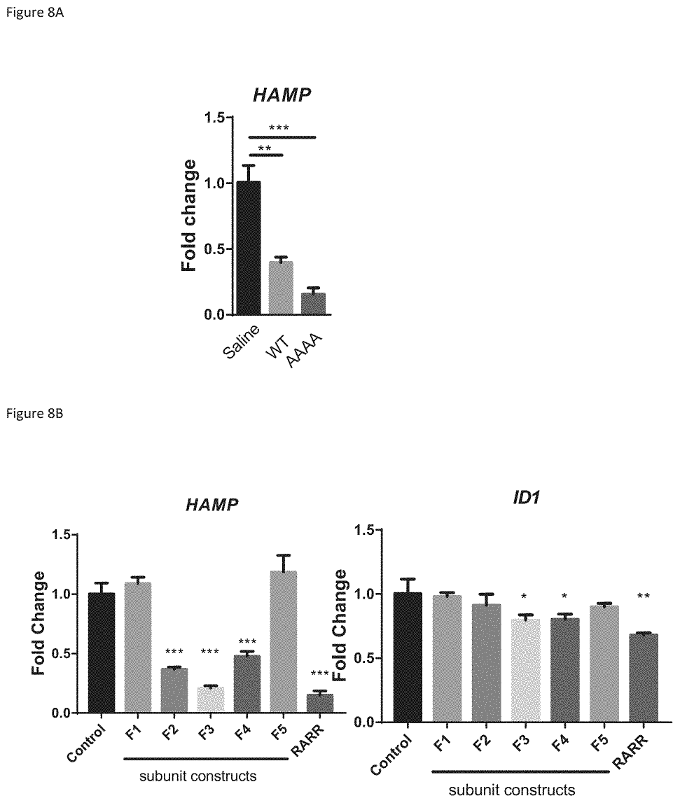

Sequence analysis of human erythroferrone using the ProP software predicted two furin cleavage sites: RARR at position 42 and RLRR at position 212 (FIG. 7A). The RLRR site is conserved in the mouse ortholog (position 198). Mutation of the RARR site to AAAA reduced the clipping of human erythroferrone after treatment with furin (FIG. 7B), arrows denote ERFE, confirming the presence of an active furin cleavage site. This monoFC-huErfe A4 construct was shown to effectively suppress hepcidin expression (FIG. 8a) To assess the potential contribution of furin cleavage to the function of ERFE, we compared the activity of the different erythroferrone subunits that could be created by furin cleavage (FIG. 8A, 8B), as well as the furin-cleavage site mutant (AAAA) and the wild-type protein. Testing of erythroferrone subunits allowed us to identify the N-terminal domain as the catalytic site of erythroferrone: only the sub-units containing that portion of the protein (F2, F3, and F4) were able to suppress HAMP and ID1. As shown previously (FIG. 6A), the subunit containing only the C1q domain (F5) was inactive.

In conclusion, in-silico design of potential furin cleavage peptides of ERFE was used to guide synthesis of the putative furin cleaved peptides, these peptides showed that the active site responsible for ERFE activity is located in the N-terminal domain.

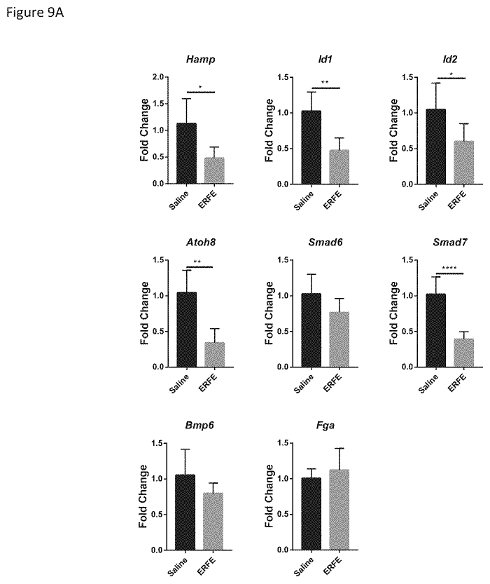

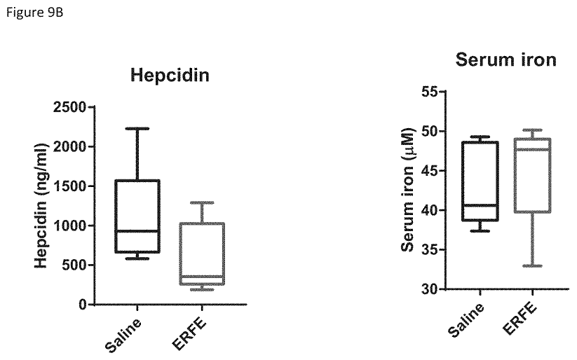

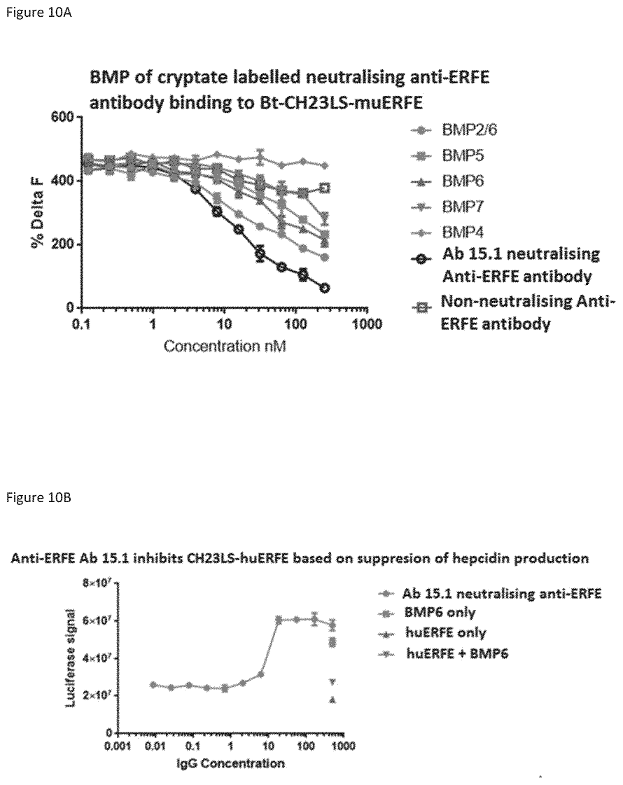

To further show that the N-terminal domain is required for the activity of erythroferrone, we injected C57BL/6 mice with the F2 subunit (containing only the N-terminal domain) and analysed the expression of Hamp and other BMP/SMAD target genes in the liver. Three hours after treatment we observed a decrease in Hamp, as well as Id1, Id2, Smad7 and Atoh8 (FIG. 9A). No changes in Fga indicate that erythroferrone injections do not cause an inflammatory reaction. We also observed a trend to a decrease in serum hepcidin protein, and no changes in serum iron at this time point (FIG. 9B).

In conclusion, the above observed gene expression pattern replicates our data observed in vitro, confirming that erythroferrone, in particular the N-terminal domain, suppresses BMP/SMD signalling leading to suppression of hepcidin.

### Example 7: Neutralising Anti-ERFE Antibodies Interfere with the ERFE BMP Interaction

(a) Investigation of the ability of BMPs to disrupt binding of cryptate-labelled anti-ERFE antibody to biotinylated murine ERFE: FRET assay was used to determine the ability of BMPs to disrupt binding between ERFE and a neutralising anti-ERFE antibody (Ab 15.1). The assay setup comprised streptavidin XL665 (cisbio assays) at a constant concentration of 1:1000 to be 50 ng per well, biotinylated monoFC-muErfe (9 μM) i.e. a monomeric FC murine ERFE fusion, cryptate labelled anti-ERFE antibody (Ab 15.1) at 15 nM. Each reaction well contained a total volume of 20 μl comprising: 5 μl streptavidin XL665, 5 μl Biotinylated monoFC-muErfe, 5 μl cryptate labelled anti-ERFE antibody 15.1, 5 μl competing antigen as a titration from 250 nM to 0.39 nM, all antigens at each concentration were assayed in duplicate. Reactions were left for 3 hours at room temperature and then absorbance at 665 nM and 615 nM was read on the Envision. BMPs 2/6 heterodimer, BMP5, BMP6 and BMP7 demonstrate measurable competition with cryptate labelled anti-ERFE antibody for binding to biotinylated monoFC-muErfe, (FIG. 10A). BMP4 did not compete effectively with the neutralising anti-ERFE antibody for binding. The assay was also conducted using a non-neutralising anti-ERFE antibody as a control.

In conclusion, BMPs, exampled here as BMPs 2/6 heterodimer, BMP5, BMP6 and BMP7 but not BMP4, bind to ERFE and can compete for binding to ERFE with a neutralising anti-ERFE antibody, the effect was not seen with the non-neutralising control anti-ERFE antibody.

(b) A neutralising anti-ERFE antibody was demonstrated to inhibit ERFE suppression of hepcidin production in a dose-dependent manner: NanoLuc cells were prepared as described in Example 1 and seeded into a multiwall dish at a density of 20000 cells/well. A neutralising anti-ERFE antibody (Ab 15.1) was serially diluted in tripling dilutions from a starting concentration of 500 nM, BMP6 was maintained at a constant concentration on 625 pM, monoFC-huErfe was maintained at a constant concentration of 20 nM, data is shown in FIG. 10B.

In conclusion, a neutralising anti-ERFE antibody can block the interaction between ERFE and BMP and prevent BMP inhibition as shown here for BMP 2/6, 5, 6 and 7 and do so in a dose dependent manner.

### Example 9: Neutralising Anti-ERFE Antibody Blocks ERFE Activity on BMPs 5/6/7 In Vitro

HUH7 cells treated with BMP 5, 6 or 7 (2 nM), murine ERFE (1 μg/ml) and anti-ERFE antibody 15.1 (10 μg/ml), alone or in combination, for 6 h. HAMP and ID1 gene expression was measured by qRT-PCR and expressed as fold-change relative to untreated cells, FIGS. 11A and 11B.

In conclusion, a neutralising anti-ERFE antibody can block the interaction between ERFE and BMP and prevent BMP inhibition as shown here for BMP5, 6 and 7 and antibody 15.1.

### Example 10: Investigation of the Effect of ERFE in Gluteal vs Abdominal Pre-Adipocytes

(a) Primary pre-adipocyte isolation. Human primary pre-adipocytes were prepared by isolation from white adipose tissue (WAT) biopsies taken under local anesthetic (1% lignocaine) by needle aspiration at the level of the umbilicus (ASAT) and from the upper-, outer-quadrant of the gluteal region (GSAT). Primary preadipocytes were isolated from ASAT and GSAT biopsies by mechanically mincing using scissors, washed twice with Hanks' buffered salt solution to remove contaminating blood, and then enzymatically digested in 1 mg/ml collagenase (Roche Applied Science) in Hanks' buffered salt solution in a shaking water bath (90 rpm) at 37° C. for 45 min. The digested tissue was centrifuged at 1000 rpm for 5 min at 4° C. The pellet containing stromal-vascular cells was resuspended in Dulbecco's modified Eagle's medium/F12 Ham's nutrient mixture (v/v, 1:1) containing 17.5 mM glucose and supplemented with 10% foetal calf serum, 0.25 ng/ml fibroblast growth factor, 2 mM glutamine, 100 units/ml penicillin and 100 μg/ml streptomycin. Cell stocks of APAD and GPAD cells were prepared and stored in liquid nitrogen for future studies.

(b) Generation of preadipocyte cell line: Human telomerase reverse transcriptase (hTERT) and human papillomavirus type 16 E7 oncoprotein (HPV16-E7) were sub-cloned into the pLenti6.3/V5-DEST lentiviral expression vector (Invitrogen) from the pBABE-neo-hTERT and pGEX2T E7 plasmids (Addgene), respectively. For the constitutive expression of hTERT and HPV16-E7 lentiviral particles were generated in 293FT producer cells using the ViraPower HiPerform Lentiviral Expression System (Invitrogen). 1° APAD and 1° GPAD cells were pre-treated with hexadimethrine bromide (8 μg/ml) and then transduced with hTERT lentiviral particles. To select preadipocytes constitutively expressing hTERT cells were cultured in the presence of blasticidin (2 μg/ml). Blasticidin treatment of non-transduced cells was used to determine the optimal lethal concentration of blasticidin. Blasticidin-resistant cells were then transduced with HPV16-E7 lentiviral particles. Expression of hTERT and HPV16-E7 was driven by the human cytomegalovirus (CMV) immediate early promoter within the pLenti6.3/V5 vector. imAPAD and imGPAD cells were cultured as described for human primary preadipocytes with the addition of blasticidin (2 μg/ml) to the culture medium.

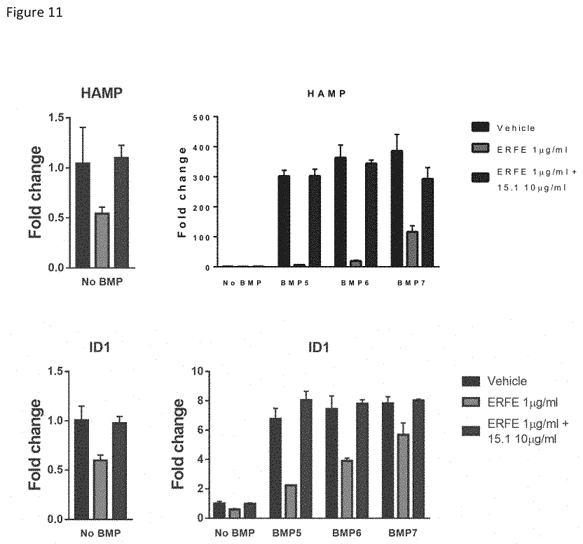

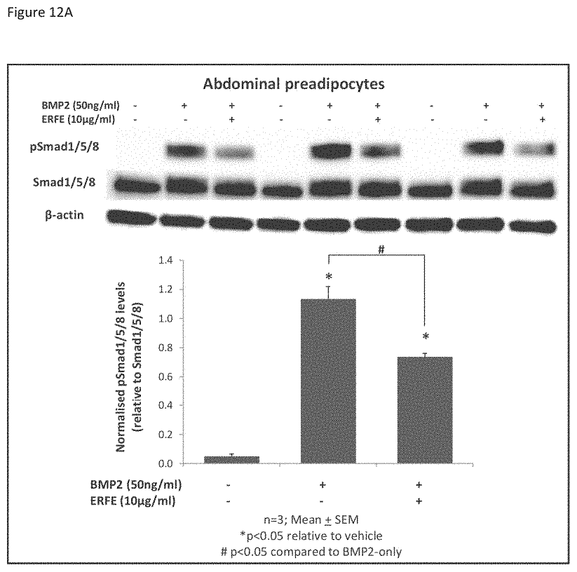

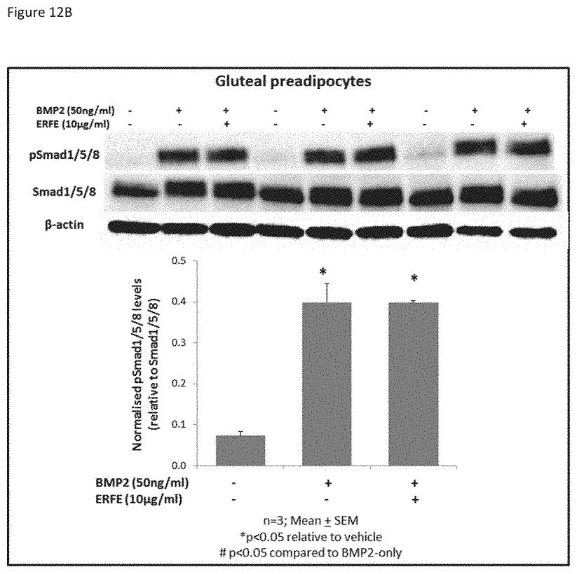

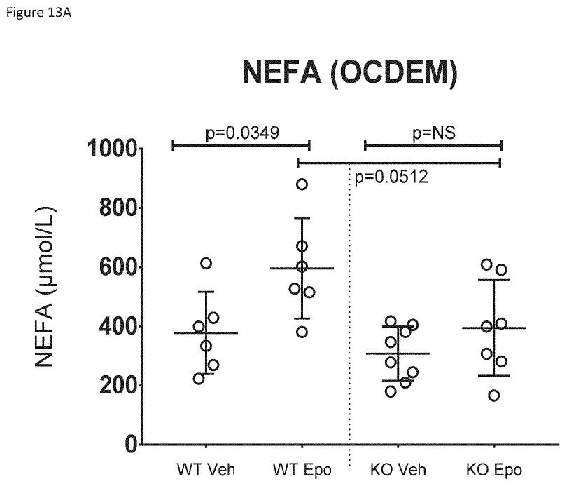

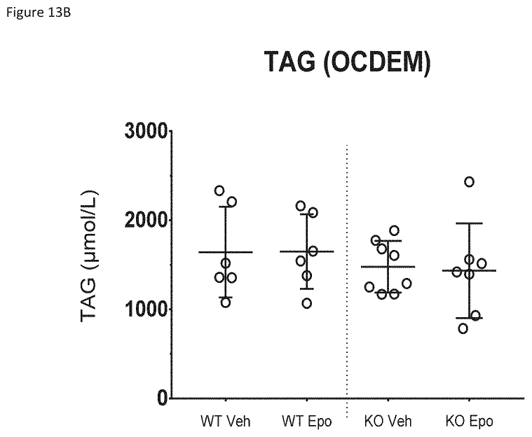

(c) Western Blot: cells were seeded at 1.5×10\hat{ }5 cells/well (6 well format), cultured for 24 hr, then incubated overnight in serum-free growth medium (Dulbecco's modified Eagle's medium/nutrient mixture F-12 Ham's (v/v, 1:1; Sigma)) supplemented with 0.25 ng/ml fibroblast growth factor (Sigma); 2 mM L-glutamine (Invitrogen); and 100 units/ml penicillin and 100 μg/ml streptomycin (Invitrogen). Cells were then incubated with BMP2 and/or mouse recombinant ERFE (or vehicle) dissolved in serum-free medium for 30 minutes, harvested for protein in 1% NP-40 lysis buffer supplemented with protease and phosphatase inhibitors before processing for Western blotting for phospho- and total SMAD1/5/8 and b-actin. Results of the Western blot are shown in FIGS. 12A and 12B.

In conclusion, treatment with BMP2 enhanced BMP signaling as observed by the increase in SMAD1/5/8 phosphorylation in both gluteal and abdominal cell types but this effect was diminished by addition of ERFE, exclusively in abdominal adipocytes. The data therefore suggest a potential effect of ERFE BMP interaction on fat distribution.

### Example 11: Investigation of the Effect of ERFE on the Release of NEFA and TAG In Vivo

Wild-type and Fam132b (ERFE) knock out male mice, on a C57BL/6 background, aged 10-13-weeks old were injected i.p. with 200 units of recombinant human erythropoietin (EPO) or water as vehicle. EPO injections have been shown to highly increase ERFE production in wild type. Mice received three injections in consecutive days (0 h, 24 h and 48 h), and were culled 24 h after the last injection (72 h). Blood was collected by cardiac puncture into Plasma Separator Tubes (BD Microtainer) and centrifuged at 8000 g for 3 min at 4 C. Each plasma was separated in two pre-cooled Eppendorf tubes for measurement of triglycerides (TAG) and non-esterified fatty acids (NEFA). Tetrahydrolipastatin was added to the NEFA tube at a final concentration 30 ug/ml to prevent lipolysis. Plasma triglyceride and NEFA concentrations were determined enzymatically using an ILab 600 Multianalyser (Instrumentation Laboratory, Warrington, U.K.). Data shown in FIGS. 13A and 13B demonstrate that treatment with EPO in WT mice significantly increased serum NEFA, while this effect was not observed in ERFE KO mice, suggesting that the release of NEFA into the blood is ERFE mediated. Increased serum NEFA have been associated with the development of obesity, diabetes and NAFLD in humans. No changes were observed in plasma TAG.

In conclusion, the present data indicates that the BMP ERFE interaction modulates serum NEFA and is implicated in the development of obesity, diabetes and NAFLD in humans.

Sequences

MAPARRPAGARLLLVYAGLLAAAAAGLGSPEPGAPSRSRARREPPPGNELPRGPGESRAGPAARPPEPTAERAHS
VDPRDAWMLFVRQSDKGVNGKKRSRGKAKKLKFGLPGPPGPPGPQGPPGPIIPPEALLKEFQLLLKGAVRQRERA
EPEPCTCGPAGPVAASLAPVSATAGEDDDDVVGDVLALLAAPLAPGPRAPRVEAAFLCRLRRDALVERRALHELGV
YYLPDAEGAFRRGPGLNLTSGQYRAPVAGFYALAATLHVALGEPPRRGPPRPRDHLRLLICIQSRCQRNASLEAIMG
LESSSELFTISVNGVLYLQMGQWTSVFLDNASGCSLTVRSGSHFSAVLLGV, SEQ ID NO: 1-[GenBank:
AHL84165.1-erythroferrone Homo sapiens]
MASRRPVGARTLLACASLLAAMGLGVPESAEPVGTHARPQPPGAELPAPPANSPPEPIAHAHSVDPRDAWMLFV
KQSDKGINSKRRSKARRLKLGLPGPPGPPGPQGPPGPFIPSEVLLKEFQLLLKGAVRQRESHLEHCTRDLPASGSPSR
VPAAQELDSQDPGALLALLAATLAQGPRAPRVEAAFHCRLRRDVQVDRRALHELGIYYLPEVEGAFHRGPGLNLTS
GQYTAPVAGFYALAATLHVALTEQPRKGPTRPRDRLRLLICIQSLCQHNASLETVMGLENSSELFISVNGVLYLQAG
HYSVFLDNASGSSLTVRSGSHFSAILLGL, SEQ ID NO: 2-[erythroferrone mouse]
AAPLAPGPRAPRVEAAFLCRLRRDALVERRALHELGVYYLPDAEGAFRRGPGLNLTSGQYRAPVAGFYALAATLHV
ALGEPPRRGPPRPRDHLRLLICIQSRCQRNASLEAIMGLESSSELFTISVNGVLYLQMGQWTSVFLDNASGCSLTVRS
GSHFSAVLLGV, SEQ ID NO: 3 , the TNF like domain, TNFD (Tissue Necrosis Factor like domain),
amino acid positions 190 to 354 of SEQ ID NO: 1.
PGPPGPQGPPGPIIPPEALLKEFQLLLKGAVRQRERAEPEPCTCGPAGPVAASLAPVSATAGEDDDDVVGDVLALL,
SEQ ID NO: 4, the NTD2 (N-terminal Domain 2), amino acid positions 114 to 189 of
SEQ ID NO: 1.
KKRSRGKAKKLKFGLPGP, SEQ ID NO: 5-CD (Collagen Domain), [amino acid positions 96 to 113]
AGLGSPEPGAPSRSRARREPPPGNELPRGPGESRAGPAARPPEPTAERAHSVDPRDAWMLFVRQSDKGVNG,
SEQ ID NO: 6-NTD1 (N-terminal Domain 1) [amino acid positions 24 to 95 of SEQ ID NO: 1]
MAPARRPAGARLLLVYAGLLAAAA, SEQ ID NO: 7-SP (Signal Peptide Domain), amino acid positions 1
to 23 of SEQ ID NO: 1.
GPRAPRVEAAF, SEQ ID NO: 8;
LLKEFQLLLKGAVRQRE, SEQ ID NO: 9;
GLPGPPGPPGPQGPPGP, SEQ ID NO: 10;
AHSVDPRDAWMLFVXQSDKGXN, SEQ ID NO: 11
AHSVDPRDAWMLFV, SEQ ID NO: 12.
AHSVDPRDAWMLFVRQSDKGVN, SEQ ID NO: 13
RDAWFVRQ [SEQ ID NO. 14]
HSVDPRDAWM [SEQ ID NO. 15]
DPRDAWFV [SEQ ID NO. 16]
DPRDAWMLFV [SEQ ID NO. 17]
Anti-ERFE antibody 15.1 Heavy Chain CDRs
CDRH1. TDYSMH [SEQ ID NO: 18]
CDRH 2. YINPNSGGTSYNQKFKG [SEQ ID NO: 19]
CDRH 3. YGYDDY [SEQ ID NO: 20]
Anti-ERFE antibody 15.1 Light Chain CDRs
CDRL1. RSSQSIVHSNGNTYLE [SEQ ID NO 21:]
CDRL 2. KVSNRFS [SEQ ID NO 22:]
CDRL 3. FQGSHVPLT [SEQ ID NO: 23]
Anti-ERFE antibody 15.1 Variable Heavy Chain-CDRs underlined
EVQLQQSGPE LVKPGASVKM SCKASGYTFT DYSMHWVKQS HGKSLEWIGY INPNSGGTSY NQKFKGKATL T
VNKSSSTAY MELRSLTSED SAVYYCVPYG YDDYWGQGTT LTVSS
[SEQ ID NO: 24]
Anti-ERFE antibody 15.1 Variable Light chain-CDRs underlined
DVLMTQTPLS LPVSLGDQVS ISCRSSQSIV HSNGNTYLEW YLQKPGQSPK LLIYKVSNRF SGVPDRFSGS GSGT
DFTLRI TRVAAEDLGV YYCFQGSHVP LTFGAGTKLE LKR [SEQ ID NO: 25]
Anti-ERFE antibody 15.1 Heavy Chain-CDRs underlined
EVQLQQSGPE LVKPGASVKM SCKASGYTFT DYSMHWVKQS HGKSLEWIGY INPNSGGTSY NQKFKGKATL T
VNKSSSTAY MELRSLTSED SAVYYCVPYG YDDYWGQGTT LTVSSAKTTA PSVYPLAPVC GDTTGSSVTL GCLVK
GYFPE PVTLTWNSGS LSSGVHTFPA VLQSDLYTLS SSVTVTSSTW PSQSITCNVA HPASSTKVDK KIEPRGPTIK
PCPPCKCPAP NLEGGPSVFI FPPKIKDVLM ISLSPIVTCV VVDVSEDDPD VQISWFVNNV EVHTAQTQTH REDY
NSTLRV VSALPIQHQD WMSGKAFACA VNNKDLPAPI ERTISKPKGS VRAPQVYVLP PPEEEMTKKQVTLTCM
VTDF MPEDIYVEWT NNGKTELNYK NTEPVLDSDG SYFMYSKLRV EKKNWVERNS YSCSVVHEGL HNHHTTKS
FS RTPG [SEQ ID NO: 26]
Anti-ERFE antibody 15.1 Light chain-CDRs underlined
DVLMTQTPLS LPVSLGDQVS ISCRSSQSIV HSNGNTYLEW YLQKPGQSPK LLIYKVSNRF SGVPDRFSGS GSGT
DFTLRI TRVAAEDLGV YYCFQGSHVP LTFGAGTKLE LKRTDAAPTV SIFPPSSEQL TSGGASVVCF LNNFYPKDIN
VKWKIDGSER QNGVLNSVVTD QDSKDSTYSM SSTLTLTKDE YERHNSYTCE ATHKTSTSPI VKSFNRNEC
[SEQ ID NO: 27]

### STATEMENTS OF INVENTION

1. The BMP agonist or antagonist for use in treating a disease of iron metabolism wherein the BMP agonist or antagonist:

(i) prevents or inhibits the activity of a BMP agonist or antagonist,

(ii) prevents or inhibits the interaction between BMP or a BMP polypeptide having BMP activity and a BMP agonist or antagonist,

(iii) prevents or inhibits the interaction between BMP or a BMP polypeptide having BMP activity and ERFE or ERFE polypeptide having erythroferrone activity,

(iv) prevents or inhibits the interaction between BMP or a BMP polypeptide having BMP activity and a BMP receptor,

(v) enhances the interaction between BMP or a BMP polypeptide having BMP activity and a BMP agonist or antagonist,

(vi) enhances the interaction between BMP or a BMP polypeptide having BMP activity and ERFE or ERFE polypeptide having erythroferrone activity,

(vii) enhances the interaction between BMP or a BMP polypeptide having BMP activity and a BMP receptor,

(vii) binds to BMP, or a BMP polypeptide having BMP activity, and prevents its interaction with and/or inhibition or activation by an agonist or antagonist,

(ix) binds to an agonist or antagonist of BMP and prevents its interaction with and/or inhibition or activation of BMP or a BMP polypeptide having BMP activity,

(x) binds to BMP, or a BMP polypeptide having BMP activity, and prevents its interaction with and/or inhibition by ERFE or ERFE polypeptide having erythroferrone activity,

(xi) binds to ERFE or an ERFE polypeptide having erythroferrone activity and prevents or inhibits its interaction with BMP or a BMP polypeptide having BMP activity and/or inhibition of BMP activity,

(xii) binds to BMP, or a BMP polypeptide having BMP activity, and enhances its interaction with and/or inhibition by ERFE or ERFE polypeptide having erythroferrone activity,

(xiii) binds to ERFE or an ERFE polypeptide having erythroferrone activity and enhances its interaction with and/or inhibition of BMP activity,

(xiv) binds to BMP or a BMP polypeptide having BMP activity and prevents or inhibits its interaction with a BMP receptor,

(xv) binds to BMP or a BMP polypeptide having BMP activity and enhances its interaction with a BMP receptor,

(xvi) binds to a BMP receptor and prevents or inhibits its interaction with BMP or BMP polypeptide having BMP activity,

(xvii) binds to a BMP receptor and enhances its interaction with BMP or BMP polypeptide having BMP activity.

2. A BMP agonist or antagonist for use according to statement 1, wherein the BMP agonist or antagonist is an agonist and wherein the agonist:

(a) inhibits the interaction between BMP or a BMP polypeptide having BMP activity and ERFE or an ERFE polypeptide having erythroferrone activity and/or inhibits the inhibition of BMP activity by ERFE or an ERFE polypeptide having erythroferrone activity,

(b) binds to BMP or a BMP polypeptide having BMP activity and prevents its interaction with and/or inhibition by an antagonist,

(c) binds to an antagonist of BMP or a BMP polypeptide having BMP activity and prevents its interaction with and/or inhibition of BMP,

(d) binds to BMP or a BMP polypeptide having BMP activity and prevents its interaction with and/or inhibition by ERFE or ERFE polypeptide having erythroferrone activity,

(e) binds to ERFE or an ERFE polypeptide having erythroferrone activity and prevents or inhibits its interaction with BMP or a BMP polypeptide having BMP activity and/or inhibition of BMP activity,

(f) binds to BMP or a BMP polypeptide having BMP activity and enhances its interaction with a BMP receptor, or

(g) binds to a BMP receptor and enhances its interaction with its BMP or BMP polypeptide having BMP activity.

3. The BMP agonist or antagonist for use according to statement 1 or 2 wherein the BMP is selected from:

(i) any one or more of BMP 2, 2/6 heterodimer, 3, 4, 5, 6, 7, 8a, 8b, 9, 10, 11, 12, 13, 14, 15,

(ii) any one or more of BMP2/6 heterodimer, BMP5, BMP6, BMP7,

(iii) any one or more of BMP5, BMP6, BMP7,

(iv) any one or more of (a) BMP2, BMP4, (b) BMP 2, (c) BMP 4, (d) BMP 5, (e) BMP 6, (f) BMP 7;

(v) any one or more of (a) BMP2, (b) BMP2/6 heterodimer, (c) BMP4, (v) BMP5, (b) BMP6 or (f) BMP7,

(vi) any one or more of (a) BMP2, (b) BMP 2/6 or (c) BMP4.

4. The BMP agonist or antagonist for use according to any of statements 1 to 3 wherein the BMP agonist or antagonist can bind specifically and/or selectively to (a) BMP or a BMP polypeptide having BMP activity, (b) an agonist or antagonist of BMP or BMP polypeptide having BMP activity, (c) ERFE or an ERFE polypeptide having erythroferrone activity, (d) a BMP receptor; optionally with a binding constant or KD of about or less than about 10, 1, 0.1, 0.01, or 0.001 nM.

5. The BMP agonist or antagonist for use according to any of statements 1 to 4 wherein the BMP agonist or antagonist can specifically and/or selectively inhibit or enhance the binding of BMP or a BMP polypeptide having BMP activity, to any one or more of (a) an agonist or antagonist of BMP or BMP polypeptide having BMP activity, (b) a BMP receptor, (c) ERFE or ERFE polypeptide having erythroferrone activity; optionally with an IC50 or inhibition constant (Ki) of about or less than about 10, 1, 0.1, 0.01, or 0.001 nM or an enhancement of activity by any of about 2, 4, 6, 8, 10, 12, 14, 16, 18, 20, 25, 30, 35, 40, 45, 50, 55, 60, 65, 70, 75, 80, 85, 90, 95, 100, 200 fold.

6. The BMP agonist for use according to any of statements 1 to 5 wherein the BMP agonist can specifically bind to (a) BMP or a BMP polypeptide having BMP activity (b) an antagonist of BMP, (c) a BMP receptor, or (d) ERFE or ERFE polypeptide having erythroferrone activity; optionally with a binding constant or KD of about or less than about 10, 1, 0.1, 0.01, or 0.001 nM.

7. The BMP agonist for use according to any of statements 1 to 6 wherein the BMP agonist or antagonist can specifically inhibit the binding of BMP or a BMP polypeptide having BMP activity, to any one or more of (a) an antagonist of BMP, (b) a BMP receptor, and/or (c) ERFE or ERFE polypeptide having erythroferrone activity; optionally with an IC50 or inhibition constant (Ki) of about or less than about 10, 1, 0.1, 0.01, or 0.001 nM.

8. The BMP agonist for use according to any of statements 1 to 7 wherein the BMP agonist can bind specifically or selectively to (a) BMP or a BMP polypeptide having BMP activity, (b) an antagonist of BMP or BMP polypeptide having BMP activity, (c) ERFE or an ERFE polypeptide having erythroferrone activity, or (d) a BMP receptor.

9. The BMP agonist for use according to any of statements 1 to 8 wherein the BMP agonist can selectively inhibit the binding of BMP or a BMP polypeptide having BMP activity, to any one or more of (a) an antagonist of BMP, (b) a BMP receptor, (c) ERFE or ERFE polypeptide having erythroferrone activity.

10. The BMP antagonist for use according to any of statements 1 to 9 wherein the disease comprises abnormally high hepcidin levels, high hepcidin activity, or abnormally low iron levels.

11. The BMP agonist for use according to any of statements 1 to 9 wherein the disease comprises abnormally low hepcidin levels, low hepcidin activity, or abnormally high iron levels.

12. The BMP agonist for use according to statement 11 wherein the disease is thalassemia.

13. The BMP agonist for use according to statement 12 wherein the thalassemia is selected from alpha-thalassemia, beta-thalassemia, delta-thalassemia, hemoglobin E/thalassemia, hemoglobin S/thalassemia, hemoglobin C/thalassemia, hemoglobin D/thalassemia.

14. The BMP agonist for use according to statement 11 wherein the disease is chronic hepatitis B, hepatitis B, hepatitis C, alcoholic liver disease, or iron overload disease.

15. A BMP agonist or antagonist for use in treating a disease of lipid or carbohydrate metabolism.

16. The BMP agonist or antagonist for use according to statement 15 wherein the BMP agonist or antagonist:

(i) prevents or inhibits the activity of a BMP agonist or antagonist,

(ii) prevents or inhibits the interaction between BMP or a BMP polypeptide having BMP activity and a BMP agonist or antagonist,

(iii) prevents or inhibits the interaction between BMP or a BMP polypeptide having BMP activity and ERFE or ERFE polypeptide having erythroferrone activity,

(iv) prevents or inhibits the interaction between BMP or a BMP polypeptide having BMP activity and a BMP receptor,

(v) enhances the interaction between BMP or a BMP polypeptide having BMP activity and a BMP agonist or antagonist,

(vi) enhances the interaction between BMP or a BMP polypeptide having BMP activity and ERFE or ERFE polypeptide having erythroferrone activity,

(vii) enhances the interaction between BMP or a BMP polypeptide having BMP activity and a BMP receptor,

(vii) binds to BMP, or a BMP polypeptide having BMP activity, and prevents its interaction with and/or inhibition or activation by an agonist or antagonist,

(ix) binds to an agonist or antagonist of BMP and prevents its interaction with and/or inhibition or activation of BMP a BMP polypeptide having BMP activity,

(x) binds to BMP, or a BMP polypeptide having BMP activity, and prevents its interaction with and/or inhibition by ERFE or ERFE polypeptide having erythroferrone activity,

(xi) binds to ERFE or an ERFE polypeptide having erythroferrone activity and prevents or inhibits its interaction with BMP or a BMP polypeptide having BMP activity and/or inhibition of BMP activity,

(xii) binds to BMP, or a BMP polypeptide having BMP activity, and enhances its interaction with and/or inhibition by ERFE or ERFE polypeptide having erythroferrone activity,

(xiii) binds to ERFE or an ERFE polypeptide having erythroferrone activity and enhances its interaction with and/or inhibition of BMP activity,

(xiv) binds to BMP or a BMP polypeptide having BMP activity and prevents or inhibits its interaction with a BMP receptor,

(xv) binds to BMP or a BMP polypeptide having BMP activity and enhances its interaction with a BMP receptor,

(xvi) binds to a BMP receptor and prevents or inhibits its interaction with BMP or BMP polypeptide having BMP activity,

(xvii) binds to a BMP receptor and enhances its interaction with BMP or BMP polypeptide having BMP activity.

17. The BMP agonist or antagonist for use according to statement 15 or 16 wherein the BMP is selected from:

(i) any one or more of BMP 2, 2/6 heterodimer, 3, 4, 5, 6, 7, 8a, 8b, 9, 10, 11, 12, 13, 14, 15,

(ii) any one or more of BMP2/6 heterodimer, BMP5, BMP6, BMP7,

(iii) any one or more of BMP5, BMP6, BMP7,

(iv) any one or more of (a) BMP2, BMP4, (b) BMP 2, (c) BMP 4, (d) BMP 5, (e) BMP 6, (f) BMP 7;

(v) any one or more of (a) BMP2, (b) BMP2/6 heterodimer, (c) BMP4, (v) BMP5, (b) BMP6 or (f) BMP7.

18. The BMP agonist or antagonist for use according to statement 15 or 16 wherein the BMP is selected from BMP2, BMP2/6 heterodimer or BMP4.

19. The BMP agonist or antagonist for use according to any of statements 15 to 18 wherein the BMP agonist or antagonist can bind specifically and/or selectively to (a) BMP or a BMP polypeptide having BMP activity, (b) an agonist or antagonist of BMP or BMP polypeptide having BMP activity, (c) ERFE or an ERFE polypeptide having erythroferrone activity, (d) a BMP receptor.

20. The BMP agonist or antagonist for use according to any of statements 15 to 19 wherein the BMP agonist can specifically and/or selectively inhibit or enhance the binding of BMP or a BMP polypeptide having BMP activity, to any one or more of (a) an agonist or antagonist of BMP or BMP polypeptide having BMP activity, (b) a BMP receptor, (c) ERFE or ERFE polypeptide having erythroferrone activity.

21. A BMP agonist or antagonist for use according to statement 15, wherein the BMP agonist or antagonist is an agonist and wherein the agonist:

(a) inhibits the interaction between BMP or a BMP polypeptide having BMP activity and ERFE or an ERFE polypeptide having erythroferrone activity and/or inhibits the inhibition of BMP activity by ERFE or an ERFE polypeptide having erythroferrone activity,

(b) binds to BMP or a BMP polypeptide having BMP activity and prevents its interaction with and/or inhibition by an antagonist,

(c) binds to an antagonist of BMP or a BMP polypeptide having BMP activity and prevents its interaction with and/or inhibition of BMP,

(d) binds to BMP or a BMP polypeptide having BMP activity and prevents its interaction with and/or inhibition by ERFE or ERFE polypeptide having erythroferrone activity,

(e) binds to ERFE or an ERFE polypeptide having erythroferrone activity and prevents or inhibits its interaction with BMP or a BMP polypeptide having BMP activity and/or inhibition of BMP activity,

(f) binds to BMP or a BMP polypeptide having BMP activity and enhances its interaction with a BMP receptor, or

(g) binds to a BMP receptor and enhances its interaction with its BMP or BMP polypeptide having BMP activity.

22. The BMP agonist for use according to statement 21 wherein the BMP is selected from:

(i) any one or more of BMP 2, 2/6 heterodimer, 3, 4, 5, 6, 7, 8a, 8b, 9, 10, 11, 12, 13, 14, 15,

(ii) any one or more of BMP2/6 heterodimer, BMP5, BMP6, BMP7,

(iii) any one or more of BMP5, BMP6, BMP7,

(iv) any one or more of (a) BMP2, BMP4, (b) BMP 2, (c) BMP 4, (d) BMP 5, (e) BMP 6, (f) BMP 7;

(v) any one or more of (a) BMP2, (b) BMP2/6 heterodimer, (c) BMP4, (v) BMP5, (b) BMP6 or (f) BMP7.

23. The BMP agonist for use according to any of statements 21 to 22 wherein the BMP agonist or antagonist can specifically bind to (a) BMP or a BMP polypeptide having BMP activity (b) an antagonist of BMP, (c) a BMP receptor, or (d) ERFE or ERFE polypeptide having erythroferrone activity; optionally with a binding constant or KD of about or less than about 10, 1, 0.1, 0.01, or 0.001 nM.

24. The BMP agonist for use according to any of statements 21 to 23 wherein the BMP agonist or antagonist can specifically inhibit the binding of BMP or a BMP polypeptide having BMP activity, to any one or more of (a) an antagonist of BMP, (b) a BMP receptor, and/or (c) ERFE or ERFE polypeptide having erythroferrone activity; optionally with an IC50 or inhibition constant (Ki) of about or less than about 10, 1, 0.1, 0.01, or 0.001 nM.

25. The BMP agonist for use according to any of statements 21 to 24 wherein the BMP agonist can bind specifically or selectively to (a) BMP or a BMP polypeptide having BMP activity, (b) an antagonist of BMP or BMP polypeptide having BMP activity, (c) ERFE or an ERFE polypeptide having erythroferrone activity, or (d) a BMP receptor.

26. The BMP agonist for use according to any of statements 21 to 25 wherein the BMP agonist can selectively inhibit the binding of BMP or a BMP polypeptide having BMP activity, to any one or more of (a) an antagonist of BMP, (b) a BMP receptor, (c) ERFE or ERFE polypeptide having erythroferrone activity.

27. The BMP agonist or antagonist for use according to any of statements 15 to 26 wherein the disease of lipid or carbohydrate metabolism is selected from non-alcoholic fatty liver disease (NAFLD), non-alcoholic steatohepatitis (NASH), pediatric nonalcoholic fatty liver disease (NAFLD), pediatric non-alcoholic steatohepatitis (NASH), obesity, diabetes type 1, diabetes type 2, gestational diabetes, or for use in treating high cholesterol or high triglycerides.

28. The BMP agonist or antagonist for use according to any of statements 1 to 27 wherein the agonist or antagonist is: (i) a small molecule, (ii) an antibody or antigen-binding portion thereof, (iii) ERFE or an ERFE polypeptide having erythroferrone activity (iv) BMP or BMP polypeptide having BMP activity (v) a BMP receptor, (vi) a nucleic acid encoding a BMP agonist or antagonist (vii) a vector comprising a nucleic acid encoding a BMP agonist or antagonist.

29. The BMP agonist or antagonist for use according to statement 28 wherein the agonist or antagonist is an antibody or antigen-binding portion thereof that binds to, specifically binds to, or selectively binds to ERFE or an ERFE polypeptide having erythroferrone activity

30. The BMP agonist or antagonist for use according to statement 28 or 29 wherein the agonist or antagonist is an antibody or antigen-binding portion thereof that binds to:

(i) the N-terminal region of ERFE,

(ii) SEQ ID NO: 3 (TNFD domain), or amino acid positions 190 to 354 of SEQ ID NO: 1,

(iii) SEQ ID NO: 4 (NTD2 domain), or amino acid positions 114 to 189 of SEQ ID NO: 1,

(iv) SEQ ID NO: 5 (Collagen Like Domain), or amino acid positions 96 to 113 of SEQ ID NO: 1,

(v) SEQ ID NO: 6 (NTD1 domain), or amino acid positions 24 to 95 of SEQ ID NO: 1,

(vi) SEQ ID NO: 7 (SP domain), or amino acid positions 1 to 23 of SEQ ID NO: 1,

(vii) a sequence consisting of amino acids 196 to 206 of SEQ ID NO:1, or the sequence set forth in SEQ ID NO: 8,

(viii) a sequence consisting of amino acids 132 to 148 of SEQ ID NO:1, or the sequence set forth in SEQ ID NO: 9,

(ix) a sequence consisting of amino acids 109 to 125 of SEQ ID NO:1, or the sequence set forth in SEQ ID NO: 10,

(x) a sequence consisting of amino acids 73 to 94 of SEQ ID NO:1, or the sequence set forth in SEQ ID NO: 11,

(xi) a sequence consisting of amino acids 73 to 85 of SEQ ID NO:1, or the sequence set forth in SEQ ID NO: 12,

(xii) a sequence consisting of or comprising all or part of the amino acid sequence RDAWFVRQ, or SEQ ID NO: 14,

(xiii) a sequence consisting of or comprising all or part of the amino acid sequence HSVDPRDAWM, or SEQ ID NO: 15,

(xiv) a sequence consisting of or comprising all or part of the amino acid sequence HSVDPRDAWM, or SEQ ID NO: 15,

(xv) a sequence consisting of or comprising all or part of the amino acid sequence RDAWFVRQ, or SEQ ID NO: 14,

(xvi) a sequence consisting of or comprising all or part of the amino acid sequence DPRDAWFV, or SEQ ID NO: 16,

(xvii) a sequence consisting of or comprising all or part of the amino acid sequence DPRDAWMLFV, or SEQ ID NO: 14,

(xviii) a sequence consisting of or comprising all or part of the amino acid sequences HSVDPRDAWM and RDAWFVRQ, or SEQ ID NO: 14 and SEQ ID NO: 15

(xix) a sequence consisting of or comprising all or part of the amino acid sequence SEQ ID NO:1 or sequence having 95 to 100% identity to SEQ ID NO: 1.

31. The BMP agonist or antagonist for use according to any of statements 28 to 30 wherein the agonist or antagonist is an antibody or antigen-binding portion thereof and wherein the antibody or antigen binding portion thereof comprises:

(i) the CDR sequences: CDRH1, SEQ ID NO: 18; CDRH2, SEQ ID NO: 19; CDRH3, SEQ ID NO: 20; CDRL1, SEQ ID NO: 21; CDRL2, SEQ ID NO: 22; CDRL3, SEQ ID NO: 23,

(ii) the VH and VL sequences, SEQ ID NO: 24, and SEQ ID NO: 25; or

(iii) the heavy and light chain sequences, SEQ ID NO: 26, and SEQ ID NO: 27.

32. The BMP agonist or antagonist for use according to statement 31 wherein the antibody or antigen-binding portion thereof (i) specifically binds to a sequence consisting of or comprising all or part of the amino acid sequence RDAWFVRQ, or SEQ ID NO: 14, (ii) specifically binds to a sequence consisting of or comprising all or part of the amino acid sequence HSVDPRDAWM, or SEQ ID NO: 15, (iii) specifically binds to a sequence consisting of or comprising all or part of the amino acid sequences HSVDPRDAWM and RDAWFVRQ, or SEQ ID NO: 14 and SEQ ID NO: 15.

33. The BMP agonist or antagonist for use according to statement 28 or 30 wherein the antibody competes for binding to ERFE or an ERFE polypeptide having erythroferrone activity with an antibody or antigen binding portion thereof of statement 31 or 32.

34. The BMP agonist or antagonist for use according to statements 28 to 30 wherein the agonist or antagonist is ERFE or is an ERFE polypeptide having erythroferrone activity wherein the ERFE polypeptide having erythroferrone activity is an N-terminal region of EFRE lacking or truncated within the C1Q region of amino acids 195 to 354 of SEQ ID NO:1; wherein the N-terminal region of EFRE comprises or consists of: (i) amino acids 1 to 212 of SEQ ID NO:1, (ii) amino acids 1 to 142 of SEQ ID NO:1, (iii) amino acids 1 to 42 of SEQ ID NO:1, (iv) amino acids 1 to 24 of SEQ ID NO:1, (v) amino acids 24 to 96 of SEQ ID NO:1, (vi) amino acids 96 to 114 of SEQ ID NO:1, (vii) amino acids 114 to 195 of SEQ ID NO:1, (viii) amino acids 1 to 96 of SEQ ID NO:1, (ix) amino acids 1 to 114 of SEQ ID NO:1, (x) amino acids 1 to 190 of SEQ ID NO:1, (xi) amino acids 1 to 195 of SEQ ID NO:1, (xii) amino acids 196 to 206 of SEQ ID NO:1, or the sequence set forth in SEQ ID NO: 8 [GPRAPRVEAAF, SEQ ID NO: 8]; (xiii) amino acids 132 to 148 of SEQ ID NO:1, or the sequence set forth in SEQ ID NO: 9, (xiv) amino acids 109 to 125 of SEQ ID NO:1, or the sequence set forth in SEQ ID NO: 10, (xv) amino acids 73 to 94 of SEQ ID NO:1, or the sequence set forth in SEQ ID NO: 11, or (xvi) amino acids 73 to 85 of SEQ ID NO:1, or the sequence set forth in SEQ ID NO: 12.

35. A pharmaceutical composition comprising the BMP agonist or antagonist for use according to any preceding statement wherein the pharmaceutical composition comprises one or more BMP agonist or antagonist and a pharmaceutically acceptable carrier and/or an excipient.

36. The BMP agonist or antagonist for use according to any of statements 1 to 34 or the pharmaceutical composition for use according to statement 35 wherein the BMP agonist or antagonist or pharmaceutical composition is provided for use separately, sequentially or simultaneously in combination with a second therapeutic agent, optionally wherein the combination is provided as a pharmaceutical composition comprising a pharmaceutically acceptable carrier and/or an excipient.

37. The BMP agonist or antagonist for use or the pharmaceutical composition for use according to statement 36 wherein the second therapeutic agent is selected from: (i) a BMP agonist or antagonist which is a small molecule, (ii) an antibody or antigen binding portion thereof which binds ERFE or an ERFE polypeptide having erythroferrone activity, (iii) an antibody or antigen binding portion thereof which binds BMP or BMP polypeptide having BMP activity, (iv) ERFE or an ERFE polypeptide having erythroferrone activity, (v) BMP or BMP polypeptide having BMP activity, (vi) a BMP receptor, (vii) a nucleic acid encoding a BMP agonist or antagonist (viii) a vector comprising a nucleic acid encoding a BMP agonist or antagonist. (ix) a nucleic acid encoding an anti-BMP or anti-ERFE antibody or vector containing said nucleic acid, (x) insulin sensitizers, (xi) metformin, (xii) thiazolidinedione, (xiii) a statin, (xiv) pentoxifylline, (xv) diuretics, (xvi) an ACE inhibitor, (xvii) simvastatin, (xviii) sitagliptin, (xix) a GLP-1 agonist, (xx) insulin, or (xxi) a synthetic insulin analog.

1. A BMP agonist for use in treating a disease of iron metabolism, wherein the BMP agonist
(a) inhibits the interaction between BMP or a BMP polypeptide having BMP activity and ERFE or an ERFE polypeptide having erythroferrone activity and/or inhibits the inhibition of BMP activity by ERFE or an ERFE polypeptide having erythroferrone activity,
(b) binds to BMP or a BMP polypeptide having BMP activity and prevents its interaction with and/or inhibition by an antagonist,
(c) binds to an antagonist of BMP or a BMP polypeptide having BMP activity and prevents its interaction with and/or inhibition of BMP,
(d) binds to BMP or a BMP polypeptide having BMP activity and prevents its interaction with and/or inhibition by ERFE or ERFE polypeptide having erythroferrone activity,
(e) binds to ERFE or an ERFE polypeptide having erythroferrone activity and prevents or inhibits its interaction with BMP or a BMP polypeptide having BMP activity and/or inhibition of BMP activity,
(f) binds to BMP or a BMP polypeptide having BMP activity and enhances its interaction with a BMP receptor, or
(g) binds to a BMP receptor and enhances its interaction with its BMP or BMP polypeptide having BMP activity.

2. The BMP agonist for use according to claim 1 wherein the BMP is selected from:
(i) any one or more of BMP 2, 2/6 heterodimer, 3, 4, 5, 6, 7, 8a, 8b, 9, 10, 11, 12, 13, 14, 15,
(ii) any one or more of BMP2/6 heterodimer, BMP5, BMP6, BMP7,
(iii) any one or more of BMP5, BMP6, BMP7,
(iv) any one or more of (a) BMP2, BMP4, (b) BMP 2, (c) BMP 4, (d) BMP 5, (e) BMP 6, (f) BMP 7;
(v) any one or more of (a) BMP2, (b) BMP2/6 heterodimer, (c) BMP4, (v) BMP5, (b) BMP6 or (f) BMP7,
(vi) any one or more of (a) BMP2, (b) BMP 2/6 or (c) BMP4.

3. The BMP agonist for use according to claim 1 or 2 wherein the BMP agonist can specifically bind to (a) BMP or a BMP polypeptide having BMP activity (b) an antagonist of BMP, (c) a BMP receptor, or (d) ERFE or ERFE polypeptide having erythroferrone activity; optionally with a binding constant or KD of about or less than about 0.001 nM.

4. The BMP agonist for use according to any of claims 1 to 3 wherein the BMP agonist can specifically inhibit the binding of BMP or a BMP polypeptide having BMP activity, to any one or more of (a) an antagonist of BMP, (b) a BMP receptor, and/or (c) ERFE or ERFE polypeptide having erythroferrone activity, optionally with an IC50 or inhibition constant (Ki) of about or less than about 0.001 nM.

5. The BMP agonist for use according to any of claims 1 to 4 wherein the BMP agonist can bind specifically or selectively to (a) BMP or a BMP polypeptide having BMP activity, (b) an antagonist of BMP or BMP polypeptide having BMP activity, (c) ERFE or an ERFE polypeptide having erythroferrone activity, or (d) a BMP receptor.

6. The BMP agonist for use according to any of claims 1 to 5 wherein the BMP agonist can selectively inhibit the binding of BMP or a BMP polypeptide having BMP activity, to any one or more of (a) an antagonist of BMP, (b) a BMP receptor, (c) ERFE or ERFE polypeptide having erythroferrone activity.

7. The BMP agonist for use according to any of claims 1 to 6 wherein the disease comprises abnormally low hepcidin levels, low hepcidin activity, or abnormally high iron levels.

8. The BMP agonist for use according to claim 7 wherein the disease is thalassemia.

9. The BMP agonist for use according to claim 8 wherein the thalassemia is selected from alpha-thalassemia, beta-thalassemia, delta-thalassemia, hemoglobin E/thalassemia, hemoglobin S/thalassemia, hemoglobin C/thalassemia, hemoglobin D/thalassemia.

10. The BMP agonist for use according to claim 7 wherein the disease is chronic hepatitis B, hepatitis B, hepatitis C, alcoholic liver disease, or iron overload disease.

11. A BMP agonist or antagonist for use in treating a disease of lipid or carbohydrate metabolism.

12. The BMP agonist or antagonist for use according to claim 11 wherein the BMP agonist or antagonist:
(i) prevents or inhibits the activity of a BMP agonist or antagonist,
(ii) prevents or inhibits the interaction between BMP or a BMP polypeptide having BMP activity and a BMP agonist or antagonist,
(iii) prevents or inhibits the interaction between BMP or a BMP polypeptide having BMP activity and ERFE or ERFE polypeptide having erythroferrone activity,
(iv) prevents or inhibits the interaction between BMP or a BMP polypeptide having BMP activity and a BMP receptor,
(v) enhances the interaction between BMP or a BMP polypeptide having BMP activity and a BMP agonist or antagonist,
(vi) enhances the interaction between BMP or a BMP polypeptide having BMP activity and ERFE or ERFE polypeptide having erythroferrone activity,
(vii) enhances the interaction between BMP or a BMP polypeptide having BMP activity and a BMP receptor,
(vii) binds to BMP, or a BMP polypeptide having BMP activity, and prevents its interaction with and/or inhibition or activation by an agonist or antagonist,
(ix) binds to an agonist or antagonist of BMP and prevents its interaction with and/or inhibition or activation of BMP or a BMP polypeptide having BMP activity,
(x) binds to BMP, or a BMP polypeptide having BMP activity, and prevents its interaction with and/or inhibition by ERFE or ERFE polypeptide having erythroferrone activity,
(xi) binds to ERFE or an ERFE polypeptide having erythroferrone activity and prevents or inhibits its interaction with BMP or a BMP polypeptide having BMP activity and/or inhibition of BMP activity,
(xii) binds to BMP, or a BMP polypeptide having BMP activity, and enhances its interaction with and/or inhibition by ERFE or ERFE polypeptide having erythroferrone activity,
(xiii) binds to ERFE or an ERFE polypeptide having erythroferrone activity and enhances its interaction with and/or inhibition of BMP activity,
(xiv) binds to BMP or a BMP polypeptide having BMP activity and prevents or inhibits its interaction with a BMP receptor,
(xv) binds to BMP or a BMP polypeptide having BMP activity and enhances its interaction with a BMP receptor,
(xvi) binds to a BMP receptor and prevents or inhibits its interaction with BMP or BMP polypeptide having BMP activity,
(xvii) binds to a BMP receptor and enhances its interaction with BMP or BMP polypeptide having BMP activity.

13. The BMP agonist or antagonist for use according to claim 11 or 12 wherein the BMP is selected from:
(i) any one or more of BMP 2, 2/6 heterodimer, 3, 4, 5, 6, 7, 8a, 8b, 9, 10, 11, 12, 13, 14, 15,
(ii) any one or more of BMP2/6 heterodimer, BMP5, BMP6, BMP7,
(iii) any one or more of BMP5, BMP6, BMP7,
(iv) any one or more of (a) BMP2, BMP4, (b) BMP 2, (c) BMP 4, (d) BMP 5, (e) BMP 6, (f) BMP 7;
(v) any one or more of (a) BMP2, (b) BMP2/6 heterodimer, (c) BMP4, (v) BMP5, (b) BMP6 or (f) BMP7.

14. The BMP agonist or antagonist for use according to claim 11 or 12 wherein the BMP is selected from BMP2, BMP2/6 heterodimer or BMP4.

15. The BMP agonist or antagonist for use according to any of claims 11 to 14 wherein the BMP agonist or antagonist can bind specifically and/or selectively to (a) BMP or a BMP polypeptide having BMP activity, (b) an agonist or antagonist of BMP or BMP polypeptide having BMP activity, (c) ERFE or an ERFE polypeptide having erythroferrone activity, (d) a BMP receptor.

16. The BMP agonist or antagonist for use according to any of claims 11 to 15 wherein the BMP agonist or antagonist can specifically and/or selectively inhibit or enhance the binding of BMP or a BMP polypeptide having BMP activity, to any one or more of (a) an agonist or antagonist of BMP or BMP polypeptide having BMP activity, (b) a BMP receptor, (c) ERFE or ERFE polypeptide having erythroferrone activity.

17. The BMP agonist or antagonist for use according to any of claims 11 to 16 wherein the disease of lipid or carbohydrate metabolism is selected from non-alcoholic fatty liver disease (NAFLD), non-alcoholic steatohepatitis (NASH), pediatric nonalcoholic fatty liver disease (NAFLD), pediatric non-alcoholic steatohepatitis (NASH), obesity, diabetes type 1, diabetes type 2, gestational diabetes, or for use in treating high cholesterol or high triglycerides.

18. The BMP agonist or antagonist for use according to any of claims 1 to 17 wherein the agonist or antagonist is: (i) a small molecule, (ii) an antibody or antigen-binding portion thereof, (iii) ERFE or an ERFE polypeptide having erythroferrone activity (iv) BMP or BMP polypeptide having BMP activity (v) a BMP receptor, (vi) a nucleic acid encoding a BMP agonist or antagonist (vii) a vector comprising a nucleic acid encoding a BMP agonist or antagonist.

19. The BMP agonist or antagonist for use according to claim 18 wherein the agonist or antagonist is an antibody or antigen-binding portion thereof that binds to, specifically binds to, or selectively binds to ERFE or an ERFE polypeptide having erythroferrone activity

20. The BMP agonist or antagonist for use according to claim 18 or 19 wherein the agonist or antagonist is an antibody or antigen-binding portion thereof that binds to:
(i) the N-terminal region of ERFE,
(ii) SEQ ID NO: 3 (TNFD domain), or amino acid positions 190 to 354 of SEQ ID NO: 1,
(iii) SEQ ID NO: 4 (NTD2 domain), or amino acid positions 114 to 189 of SEQ ID NO: 1,
(iv) SEQ ID NO: 5 (Collagen Like Domain), or amino acid positions 96 to 113 of SEQ ID NO: 1,
(v) SEQ ID NO: 6 (NTD1 domain), or amino acid positions 24 to 95 of SEQ ID NO: 1,
(vi) SEQ ID NO: 7 (SP domain), or amino acid positions 1 to 23 of SEQ ID NO: 1,
(vii) a sequence consisting of amino acids 196 to 206 of SEQ ID NO:1, or the sequence set forth in SEQ ID NO: 8,
(viii) a sequence consisting of amino acids 132 to 148 of SEQ ID NO:1, or the sequence set forth in SEQ ID NO: 9,
(ix) a sequence consisting of amino acids 109 to 125 of SEQ ID NO:1, or the sequence set forth in SEQ ID NO: 10,
(x) a sequence consisting of amino acids 73 to 94 of SEQ ID NO:1, or the sequence set forth in SEQ ID NO: 11,
(xi) a sequence consisting of amino acids 73 to 85 of SEQ ID NO:1, or the sequence set forth in SEQ ID NO: 12,
(xii) a sequence consisting of or comprising all or part of the amino acid sequence RDAWFVRQ, or SEQ ID NO: 14,
(xiii) a sequence consisting of or comprising all or part of the amino acid sequence HSVDPRDAWM, or SEQ ID NO: 15,
(xiv) a sequence consisting of or comprising all or part of the amino acid sequence HSVDPRDAWM, or SEQ ID NO: 15,
(xv) a sequence consisting of or comprising all or part of the amino acid sequence RDAWFVRQ, or SEQ ID NO: 14,
(xvi) a sequence consisting of or comprising all or part of the amino acid sequence DPRDAWFV, or SEQ ID NO: 16,
(xvii) a sequence consisting of or comprising all or part of the amino acid sequence DPRDAWMLFV, or SEQ ID NO: 17,
(xviii) a sequence consisting of or comprising all or part of the amino acid sequences HSVDPRDAWM and RDAWFVRQ, or SEQ ID NO: 14 and SEQ ID NO: 15,
(xix) a sequence consisting of or comprising all or part of the amino acid sequence SEQ ID NO:1 or sequence having 95 to 100% identity to SEQ ID NO: 1.

21. The BMP agonist or antagonist for use according to any of claims 18 to 20 wherein the agonist or antagonist is an antibody or antigen-binding portion thereof and wherein the antibody or antigen binding portion thereof comprises:
(i) the CDR sequences: CDRH1, SEQ ID NO: 18; CDRH2, SEQ ID NO: 19; CDRH3, SEQ ID NO: 20; CDRL1, SEQ ID NO: 21; CDRL2, SEQ ID NO: 22; CDRL3, SEQ ID NO: 23,
(ii) the VH and VL sequences, SEQ ID NO: 24, and SEQ ID NO: 25; or
(iii) the heavy and light chain sequences, SEQ ID NO: 26, and SEQ ID NO: 27.

22. The BMP agonist or antagonist for use according to claim 21 wherein the antibody or antigen-binding portion thereof (i) specifically binds to a sequence consisting of or comprising all or part of the amino acid sequence RDAWFVRQ, or SEQ ID NO: 14, (ii) specifically binds to a sequence consisting of or comprising all or part of the amino acid sequence HSVDPRDAWM, or SEQ ID NO: 15, (iii) specifically binds to a sequence consisting of or comprising all or part of the amino acid sequences HSVDPRDAWM and RDAWFVRQ, or SEQ ID NO: 14 and SEQ ID NO: 15.

23. The BMP agonist or antagonist for use according to claim 18 or 20 wherein the antibody competes for binding to ERFE or an ERFE polypeptide having erythroferrone activity with an antibody or antigen binding portion thereof of claim 21 or 22.

24. The BMP agonist or antagonist for use according to claim 18 wherein the agonist or antagonist is ERFE or is an ERFE polypeptide having erythroferrone activity wherein the ERFE polypeptide having erythroferrone activity is an N-terminal region of EFRE lacking or truncated within the C1Q region of amino acids 195 to 354 of SEQ ID NO:1; wherein the N-terminal region of EFRE comprises or consists of: (i) amino acids 1 to 212 of SEQ ID NO:1, (ii) amino acids 1 to 142 of SEQ ID NO:1, (iii) amino acids 1 to 42 of SEQ ID NO:1, (iv) amino acids 1 to 24 of SEQ ID NO:1, (v) amino acids 24 to 96 of SEQ ID NO:1, (vi) amino acids 96 to 114 of SEQ ID NO:1, (vii) amino acids 114 to 195 of SEQ ID NO:1, (viii) amino acids 1 to 96 of SEQ ID NO:1, (ix) amino acids 1 to 114 of SEQ ID NO:1, (x) amino acids 1 to 190 of SEQ ID NO:1, (xi) amino acids 1 to 195 of SEQ ID NO:1, (xii) amino acids 196 to 206 of SEQ ID NO:1, or the sequence set forth in SEQ ID NO: 8 [GPRAPRVEAAF, SEQ ID NO: 8]; (xiii) amino acids 132 to 148 of SEQ ID NO:1, or the sequence set forth in SEQ ID NO: 9, (xiv) amino acids 109 to 125 of SEQ ID NO:1, or the sequence set forth in SEQ ID NO: 10, (xv) amino acids 73 to 94 of SEQ ID NO:1, or the sequence set forth in SEQ ID NO: 11, or (xvi) amino acids 73 to 85 of SEQ ID NO:1, or the sequence set forth in SEQ ID NO: 12.

25. A pharmaceutical composition comprising the BMP agonist or antagonist for use according to any preceding claim wherein the pharmaceutical composition comprises one or more BMP agonist or antagonist and a pharmaceutically acceptable carrier and/or an excipient.

26. The BMP agonist or antagonist for use according to any of claims 1 to 24 or the pharmaceutical composition for use according to claim 25 wherein the BMP agonist or antagonist or pharmaceutical composition is provided for use separately, sequentially or simultaneously in combination with a second therapeutic agent, optionally wherein the combination is provided as a pharmaceutical composition comprising a pharmaceutically acceptable carrier and/or an excipient.

27. The BMP agonist or antagonist for use or the pharmaceutical composition for use according to claim 26 wherein the second therapeutic agent is selected from: (i) a BMP agonist or antagonist which is a small molecule, (ii) an antibody or antigen binding portion thereof which binds ERFE or an ERFE polypeptide having erythroferrone activity, (iii) an antibody or antigen binding portion thereof which binds BMP or BMP polypeptide having BMP activity, (iv) ERFE or an ERFE polypeptide having erythroferrone activity, (v) BMP or BMP polypeptide having BMP activity, (vi) a BMP receptor, (vii) a nucleic acid encoding a BMP agonist or antagonist (viii) a vector comprising a nucleic acid encoding a BMP agonist or antagonist. (ix) a nucleic acid encoding an anti-BMP or anti-ERFE antibody or vector containing said nucleic acid, (x) insulin sensitizers, (xi) metformin, (xii) thiazolidinedione, (xiii) a statin, (xiv) pentoxifylline, (xv) diuretics, (xvi) an ACE inhibitor, (xvii) simvastatin, (xviii) sitagliptin, (xix) a GLP-1 agonist, (xx) insulin, or (xxi) a synthetic insulin analog.


SEQUENCE LISTING (27 sequences)

SEQ ID NO: 1 - protein, 354 aa, Homo sapiens
MAPARRPAGARLLLVYAGLLAAAAAGLGSPEPGAPSRSRARREPPPGNELPRGPGESRAGPAARPPEPTAERAHSVDPRDAWMLFVRQSDKGVNGKKRSRGKAKKLKFGLPGPPGPPGPQGPPGPIIPPEALLKEFQLLLKGAVRQRERAEPEPCTCGPAGPVAASLAPVSATAGEDDDDVVGDVLALLAAPLAPGPRAPRVEAAFLCRLRRDALVERRALHELGVYYLPDAEGAFRRGPGLNLTSGQYRAPVAGFYALAATLHVALGEPPRRGPPRPRDHLRLLICIQSRCQRNASLEAIMGLESSSELFTISVNGVLYLQMGQWTSVFLDNASGCSLTVRSGSHFSAVLLGV

SEQ ID NO: 2 - protein, 334 aa, Mus musculus
MASRRPVGARTLLACASLLAAMGLGVPESAEPVGTHARPQPPGAELPAPPANSPPEPIAHAHSVDPRDAWMLFVKQSDKGINSKRRSKARRLKLGLPGPPGPPGPQGPPGPFIPSEVLLKEFQLLLKGAVRQRESHLEHCTRDLPASGSPSRVPAAQELDSQDPGALLALLAATLAQGPRAPRVEAAFHCRLRRDVQVDRRALHELGIYYLPEVEGAFHRGPGLNLTSGQYTAPVAGFYALAATLHVALTEQPRKGPTRPRDRLRLLICIQSLCQHNASLETVMGLENSSELFISVNGVLYLQAGHYSVFLDNASGSSLTVRSGSHFSAILLGL

SEQ ID NO: 3 - protein, 165 aa, Artificial Sequence
  Synthetic Construct
AAPLAPGPRAPRVEAAFLCRLRRDALVERRALHELGVYYLPDAEGAFRRGPGLNLTSGQYRAPVAGFYALAATLHVALGEPPRRGPPRPRDHLRLLICIQSRCQRNASLEAIMGLESSSELFTISVNGVLYLQMGQWTSVFLDNASGCSLTVRSGSHFSAVLLGV

SEQ ID NO: 4 - protein, 76 aa, Artificial Sequence
  Synthetic Construct
PGPPGPQGPPGPIIPPEALLKEFQLLLKGAVRQRERAEPEPCTCGPAGPVAASLAPVSATAGEDDDDVVGDVLALL

SEQ ID NO: 5 - protein, 18 aa, Artificial Sequence
  Synthetic Construct
KKRSRGKAKKLKFGLPGP

SEQ ID NO: 6 - protein, 71 aa, Artificial Sequence
  Synthetic Construct
AGLGSPEPGAPSRSRARREPPPGNELPRGPGESRAGPAARPPEPTAERAHSVDPRDAWMLFVRQSDKGVNG

SEQ ID NO: 7 - protein, 24 aa, Artificial Sequence
  Synthetic Construct
MAPARRPAGARLLLVYAGLLAAAA

SEQ ID NO: 8 - protein, 11 aa, Artificial Sequence
  Synthetic Construct
GPRAPRVEAAF

SEQ ID NO: 9 - protein, 17 aa, Artificial Sequence
  Synthetic Construct
LLKEFQLLLKGAVRQRE

SEQ ID NO: 10 - protein, 17 aa, Artificial Sequence
  Synthetic Construct
GLPGPPGPPGPQGPPGP

SEQ ID NO: 11 - protein, 22 aa, Artificial Sequence
  Synthetic Construct
  misc_feature (15)..(15): Xaa can be any naturally occurring amino acid
  misc_feature (21)..(21): Xaa can be any naturally occurring amino acid
AHSVDPRDAWMLFVXQSDKGXN

SEQ ID NO: 12 - protein, 14 aa, Artificial Sequence
  Synthetic Construct
AHSVDPRDAWMLFV

SEQ ID NO: 13 - protein, 22 aa, Artificial Sequence
  Synthetic Construct
AHSVDPRDAWMLFVRQSDKGVN

SEQ ID NO: 14 - protein, 8 aa, Artificial Sequence
  Synthetic Construct
RDAWFVRQ

SEQ ID NO: 15 - protein, 10 aa, Artificial Sequence
  Synthetic Construct
HSVDPRDAWM

SEQ ID NO: 16 - protein, 8 aa, Artificial Sequence
  Synthetic Construct
DPRDAWFV

SEQ ID NO: 17 - protein, 10 aa, Artificial Sequence
  Synthetic Construct
DPRDAWMLFV

SEQ ID NO: 18 - protein, 6 aa, Artificial Sequence
  Synthetic Construct
TDYSMH

SEQ ID NO: 19 - protein, 17 aa, Artificial Sequence
  Synthetic Construct
YINPNSGGTSYNQKFKG

SEQ ID NO: 20 - protein, 6 aa, Artificial Sequence
  Synthetic Construct
YGYDDY

SEQ ID NO: 21 - protein, 16 aa, Artificial Sequence
  Synthetic Construct
RSSQSIVHSNGNTYLE

SEQ ID NO: 22 - protein, 7 aa, Artificial Sequence
  Synthetic Construct
KVSNRFS

SEQ ID NO: 23 - protein, 9 aa, Artificial Sequence
  Synthetic Construct
FQGSHVPLT

SEQ ID NO: 24 - protein, 115 aa, Artificial Sequence
  Synthetic Construct
EVQLQQSGPELVKPGASVKMSCKASGYTFTDYSMHWVKQSHGKSLEWIGYINPNSGGTSYNQKFKGKATLTVNKSSSTAYMELRSLTSEDSAVYYCVPYGYDDYWGQGTTLTVSS

SEQ ID NO: 25 - protein, 113 aa, Artificial Sequence
  Synthetic Construct
DVLMTQTPLSLPVSLGDQVSISCRSSQSIVHSNGNTYLEWYLQKPGQSPKLLIYKVSNRFSGVPDRFSGSGSGTDFTLRITRVAAEDLGVYYCFQGSHVPLTFGAGTKLELKR

SEQ ID NO: 26 - protein, 444 aa, Artificial Sequence
  Synthetic Construct
EVQLQQSGPELVKPGASVKMSCKASGYTFTDYSMHWVKQSHGKSLEWIGYINPNSGGTSYNQKFKGKATLTVNKSSSTAYMELRSLTSEDSAVYYCVPYGYDDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 27 - protein, 219 aa, Artificial Sequence
  Synthetic Construct
DVLMTQTPLSLPVSLGDQVSISCRSSQSIVHSNGNTYLEWYLQKPGQSPKLLIYKVSNRFSGVPDRFSGSGSGTDFTLRITRVAAEDLGVYYCFQGSHVPLTFGAGTKLELKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC